SEQUENCE LISTING (64 sequences)

SEQ ID NO: 1 - protein, 6 aa, Homo sapiens
KLVFFA

SEQ ID NO: 2 - protein, 6 aa, Homo sapiens
VQIVYK

SEQ ID NO: 3 - protein, 6 aa, Homo sapiens
KLVFFG

SEQ ID NO: 4 - protein, 7 aa, Homo sapiens
KLVFFAK

SEQ ID NO: 5 - protein, 7 aa, Homo sapiens
KLVFFAG

SEQ ID NO: 6 - protein, 8 aa, Homo sapiens
KLVFFAEN

SEQ ID NO: 7 - protein, 10 aa, Homo sapiens
KLVFFAENVG

SEQ ID NO: 8 - protein, 13 aa, Homo sapiens
KLVFFAGNVGSNK

SEQ ID NO: 9 - protein, 7 aa, Homo sapiens
GDVGSNK

SEQ ID NO: 10 - protein, 7 aa, Homo sapiens
QDVGSNK

SEQ ID NO: 11 - protein, 6 aa, Homo sapiens
GGVVIA

SEQ ID NO: 12 - protein, 24 aa, Homo sapiens
LVFFAEDVGSNKGAIIGLMVGGVV

SEQ ID NO: 13 - protein, 26 aa, Homo sapiens
LVFFAEDVGSNKGAIIGLMVGGVVIA

SEQ ID NO: 14 - protein, 6 aa, Homo sapiens
GVVEVD

SEQ ID NO: 15 - protein, 6 aa, Homo sapiens
GDVIEV

SEQ ID NO: 16 - protein, 6 aa, Homo sapiens
SSTNVG

SEQ ID NO: 17 - protein, 7 aa, Homo sapiens
GNNQQNY

SEQ ID NO: 18 - protein, 8 aa, Homo sapiens
NFGAILSS

SEQ ID NO: 19 - protein, 11 aa, Homo sapiens
SSNNFGAILSS

SEQ ID NO: 20 - protein, 10 aa, Homo sapiens
SNNFGAILSS

SEQ ID NO: 21 - protein, 42 aa, Homo sapiens [sequence omitted]

SEQ ID NO: 22 - protein, 758 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 23 - protein, 6 aa, Homo sapiens
NKGAII

SEQ ID NO: 24 - protein, 6 aa, Homo sapiens
GAIIGL

SEQ ID NO: 25 - protein, 6 aa, Homo sapiens
AIIGLM

SEQ ID NO: 26 - protein, 8 aa, Homo sapiens
MVGGVVIA

SEQ ID NO: 27 - protein, 6 aa, Homo sapiens
MVGGVV

SEQ ID NO: 28 - protein, 6 aa, Homo sapiens
GGVVIA

SEQ ID NO: 29 - protein, 6 aa, Homo sapiens
VQIINK

SEQ ID NO: 30 - protein, 6 aa, Homo sapiens
GVTTVA

SEQ ID NO: 31 - protein, 6 aa, Homo sapiens
GVATVA

SEQ ID NO: 32 - protein, 7 aa, Homo sapiens
VVTGVTA

SEQ ID NO: 33 - protein, 7 aa, Homo sapiens
TGVTAVA

SEQ ID NO: 34 - protein, 6 aa, Homo sapiens
VEALYL

SEQ ID NO: 35 - protein, 6 aa, Homo sapiens
LYQLEN

SEQ ID NO: 36 - protein, 6 aa, Homo sapiens
IFQINS

SEQ ID NO: 37 - protein, 6 aa, Homo sapiens
TFQINS

SEQ ID NO: 38 - protein, 7 aa, Homo sapiens
NNFGAIL

SEQ ID NO: 39 - protein, 6 aa, Homo sapiens
SSTNVG

SEQ ID NO: 40 - protein, 6 aa, Homo sapiens
TITTLE

SEQ ID NO: 41 - protein, 7 aa, Homo sapiens
LTITTLE

SEQ ID NO: 42 - protein, 6 aa, Homo sapiens
NHVTLS

SEQ ID NO: 43 - protein, 7 aa, Homo sapiens
NHVTLSQ

SEQ ID NO: 44 - protein, 6 aa, Homo sapiens
KDWSFY

SEQ ID NO: 45 - protein, 7 aa, Homo sapiens
TIAALLS

SEQ ID NO: 46 - protein, 6 aa, Homo sapiens
AADTWE

SEQ ID NO: 47 - protein, 6 aa, Homo sapiens
YTIAAL

SEQ ID NO: 48 - skipped

SEQ ID NO: 49 - protein, 7 aa, Homo sapiens
GVIGIAQ

SEQ ID NO: 50 - protein, 7 aa, Homo sapiens
GVTGIAQ

SEQ ID NO: 51 - protein, 7 aa, Homo sapiens
DSVISLS

SEQ ID NO: 52 - protein, 8 aa, Homo sapiens
VQGIINFE

SEQ ID NO: 53 - protein, 6 aa, Homo sapiens
GTHSQW

SEQ ID NO: 54 - protein, 7 aa, Homo sapiens
GTHSQWN

SEQ ID NO: 55 - protein, 6 aa, Homo sapiens
AGAAAA

SEQ ID NO: 56 - protein, 6 aa, Homo sapiens
GAVVGG

SEQ ID NO: 57 - protein, 6 aa, Homo sapiens
GYMLGS

SEQ ID NO: 58 - protein, 6 aa, Homo sapiens
GYVLGS

SEQ ID NO: 59 - protein, 6 aa, Homo sapiens
IIHFGS

SEQ ID NO: 60 - protein, 6 aa, Homo sapiens
NQVYYR

SEQ ID NO: 61 - protein, 6 aa, Homo sapiens
PMDEYS

SEQ ID NO: 62 - protein, 6 aa, Homo sapiens
SNQNNF

SEQ ID NO: 63 - protein, 6 aa, Homo sapiens
NQNNFV

SEQ ID NO: 64 - protein, 6 aa, Homo sapiens
QHTVTT

(19) United States
(12) Patent Application Publication
(10) Pub. No.: US 2014/0154180 A1
(43) Pub. Date: Jun. 5, 2014

(54) Pharmacophores for Amyloid Fibers Involved in Alzheimer's Disease

(71) Applicants: David S. Eisenberg, Los Angeles, CA (US); Lin Jiang, Los Angeles, CA (US); Meytal Landau, Haifa (IL); Cong Liu, Los Angeles, CA (US)

(72) Inventors: David S. Eisenberg, Los Angeles, CA (US); Lin Jiang, Los Angeles, CA (US); Meytal Landau, Haifa (IL); Cong Liu, Los Angeles, CA (US)

(73) Assignee: The Regents of the University of California, Oakland, CA (US)

(21) Appl. No.: 14/232,866

(22) PCT Filed: Jul. 16, 2012
(86) PCT No.: PCT/US2012/046945

§ 371 Date: Jan. 14, 2014

Related U.S. Application Data
(60) Provisional application No. 61/507,810, filed on Jul. 14, 2011.

(51) Int. Cl. G06F 19/16; A61K 31/12; G01N 33/68; A61K 31/473; A61K 31/704; A61K 31/655; A61K 31/536

(52) U.S. Cl. CPC G06F 19/16; A61K 31/655; A61K 31/12; A61K 31/536; A61K 31/473; A61K 31/704; G01N 33/6896
USPC 424/1.65; 514/150; 514/680; 514/229.8; 514/289; 514/33; 435/7.1; 436/501; 435/375; 703/11

(57) ABSTRACT

This invention relates, e.g., to a method for designing or selecting on a computer a candidate small molecule amyloid binder or inhibitor, comprising: a) docking test compounds to the binding site or binding surface determined from the three-dimensional structure of a co-crystal of a protofilament of an amyloid protein bound to a small molecule which is known to bind to the amyloid protein, and (b) selecting test compounds which exhibit an energy below that of the small molecule used to form the co-crystal made in a), as candidate amyloid binders.

This application claims the benefit of the filing date of U.S. Provisional Application 61/507,810, filed Jul. 14, 2011, which is incorporated by reference in its entirety herein

This invention was made with Government support under Grants No. AG029430 and AG016570, awarded by the National Institutes of Health. The Government has certain rights in this invention.

### SEQUENCE LISTING

The instant application contains a Sequence Listing which has been submitted via paper and CD-R format and is hereby incorporated by reference in its entirety. Hexamer fiber-forming segments of Aβ and tau having the sequences KLVFFA (SEQ ID NO:1) and VQIVYK (SEQ ID NO:2), respectively, are referred to throughout this application. The sequence of the Aβ used in the present experiments is represented by SEQ ID NO:21. The sequence of tau is represented by SEQ ID NO:22.

### BACKGROUND INFORMATION

The devastating and incurable dementia known as Alzheimer's disease affects the thinking, memory, and behavior of dozens of millions of people worldwide. The challenge of developing chemical interventions for Alzheimer's disease has proceeded in a virtual vacuum of information about the three-dimensional structures of the two proteins most widely accepted as being involved in the etiology. These are amyloid-beta (Aβ, sometimes referred to herein as Abeta) and tau [1,2]. Both convert from largely natively disordered, soluble forms to toxic oligomers and fibers [2,3] that may be related in structure [4]. Indeed, analogs of the well-established ligands to amyloid fibers, congo-red and thioflavin T, also bind Aβ oligomers labeling them in vitro and in vivo [5]. Screens of chemical libraries have uncovered dozens of small molecules that interact with amyloid [6-8]. Curcumin and various antibiotics are a few of many fiber inhibitors that also inhibit oligomer formation [7,9,10], supporting a common underlying structure in fibers and oligomers. Despite this progress, until now there have been no atomic-level structures showing how small molecules bind to amyloid and, consequently, no means for structure-based design of specific binders.

More is known about the molecular structure of amyloid fibers, both those associated with Alzheimer's disease and with the numerous other amyloid conditions [11-15]. Common to all amyloid fibers is their X-ray fiber-diffraction pattern, with two orthogonal reflections at about 4.8 Å and 10 Å spacing suggesting a “cross-β structure” [16,17]. The determination of the first amyloid-like atomic structures revealed a motif consisting of a pair of tightly mated β-sheets, called a “steric zipper,” which is formed from a short self-complementary segment of the amyloid-forming protein [12,18,19]. The steric zipper structures elucidate the atomic features that give rise to the common cross-β diffraction pattern, corresponding to the 4.8 Å spacing between strands forming β-sheets and the ˜10 Å spacing between two mating β-sheets. The structures imply that stacks of identical short segments form the “cross-β spine” of the protofilament, the basic unit of the mature fiber, while the rest of the protein adopts either native-like or unfolded conformations peripheral to the spine [12,20].

The short segments forming steric zippers, when isolated from the rest of the protein, form well-ordered fibers on their own, with essentially all properties of the fibers of their full-length parent proteins [21,22]. These properties include similar fiber diameters and helical pitch, similar cross-β diffraction patterns, similar fiber-seeding capacities, similar stability, and similar dye binding. That stacked short amyloidogenic segments can constitute the entire spine of an amyloid-like fiber has been demonstrated with the enzyme RNase A, containing an insert of a short amyloidogenic segment [20,23]. These RNase A fibers retain enzymatic activity, showing that native-like structure remains intact with only the stacked segments forming the spine. Thus while short amyloidogenic segments cannot recapitulate the entire complexity of their parent proteins, they nonetheless serve as good models for full amyloid fibers [24] and offer the informational advantage that they often grow into microcrystals whose atomic structures can be determined [12]. To date, structures for over 90 such steric zippers have been determined from a variety of disease-associated proteins ([18,19,25-27] and Colletier et al. (2011) Molecular basis for amyloid-beta polymorphism, Proc. Natl. Acad. Sci. USA 108, 16938-43).

### DESCRIPTION OF THE DRAWINGS

The patent or application file contains at least one drawing executed in color. It is noted that many of these color drawings are present in the publication, Landau et al. (2011), Towards a Pharmacophore for Amyloid, PLoS Biol 9(6): e1001080. doi:10.1371.

FIG. 1 shows the crystal structure of the KLVFFA (SEQ ID NO:1) segment from Aβ complexed with the small molecule orange-G. (A-B) The KLVFFA (SEQ ID NO: 1) segments are packed as pairs of β-sheets forming the basic unit of the fiber, namely the steric zipper [12,18]. Here 10 layers of β-strands are depicted; actual fibers contain ˜100,000 layers. Orange-G (orange carbons) wedges open the zipper and binds between the pair of O-sheets. KLVFFA (SEQ ID NO: 1) and orange-G are shown as sticks with non-carbon atoms colored by atom type. The β-sheets are composed of anti-parallel strands (cartoon arrows), alternately colored white and blue. In panel A, the view looks down the fiber axis. In panel B, the view is perpendicular to the fiber axis; the β-strands run horizontally. The sulfonic acid groups of orange-G form salt links (pink lines) with four lysine residues, two protruding from each facing β-sheet and with a water molecule shown as an aqua sphere. Only side chain atoms are shown. The unit cell dimension of the crystal along the fiber axis (9.54 Å) is indicated. (C) Micro-crystals of KLVFFA (SEQ ID NO: 1) co-crystallized with orange-G. FIG. 9 shows details of the interactions of orange-G with the KLVFFA (SEQ ID NO: 1) peptides around it.

FIG. 2 shows orange-G binding between the two beta sheets of the steric zipper formed by the KLVFFA (SEQ ID NO: 1) segment from Aβ (residues 16-21 of Aβ). Orange-G also contacts the lysine residues in the adjacent zipper. Peptide segments, forming β-sheet structures, are shown as arrows and sticks, colored by atom type with carbons in white. Orange-G carbons are in orange for one molecule and brown for the other molecule. Surface is shown for peptide atoms contacting the orange-G molecule with the orange carbons, shown as spheres. The view in (A) looks down the fiber axis. The view in (B) is perpendicular to the fiber axis. Only side chains of interacting residues are shown. The solvent-accessible surface area of the fiber buried by orange-G (See Example IA) is 271 Å² and 272 Å² for the orange and brown colored orange-G, respectively, and is about 80% hydrophobic (contributed by the side chains of Leu17, Val18, Phe19, and Phe20). The polar interactions are contributed by the charged side chains of Lys16. In (B), one of the β-sheets from the adjacent pair and one orange-G molecule are removed for clarity.

FIG. 3 shows small molecule binding is specific for fiber polymorphism. Three forms of the KLVFFA (SEQ ID NO: 1) segment from Aβ (A-C) and the VQIVYK (SEQ ID NO: 2) segment from the tau protein (D-F) are presented. These forms serve as examples of packing polymorphism observed for amyloid fibrils [25]. The view looks down the fiber axis; three layers of depth are depicted. The peptide segments and the small molecules are shown as sticks with non-carbon atoms colored by atom type. In the KLVFFA (SEQ ID NO: 1) forms (A-C), the anti-parallel strands (cartoon arrows) are alternately colored white and blue. The VQIVYK (SEQ ID NO: 2) forms (D-F) pack in parallel β-sheets (represented as cartoon arrows with white carbons). KLVFFA (SEQ ID NO: 1) Form-1 (A) and Form-2 (B) (Colletier et al, supra) and VQIVYK (SEQ ID NO: 2) Form-1 (D) [18] are tightly packed such that there are no voids to accommodate binding of small molecules. VQIVYK (SEQ ID NO: 2) Form-2 (E) [25] shows a shift in the steric zipper generating a void that can accommodate the binding of apolar molecules such as curcumin and DDNP. A docked model of curcumin binding is shown (colored magenta) (See Example IA). Orange-G binds to unique forms of both KLVFFA (SEQ ID NO: 1) and VQIVYK (SEQ ID NO: 2) (C and F), in which large voids are present in the crystal packing. In the KLVFFA (SEQ ID NO: 1) complex (C), the binding is internal to the steric zipper, whereas in the VQIVYK (SEQ ID NO: 2) complex (F), the binding is between pairs of (β-sheets, i.e., internal to bundles of protofilaments.

FIG. 4 shows the crystal structure of the VQIVYK (SEQ ID NO: 2) segment from the tau protein complexed with orange-G. (A-C) The VQIVYK (SEQ ID NO: 2) segments pack in parallel, in-register β-sheets (cartoon arrows) that form steric zippers (two zippers are shown in panel A). Nine layers of the fiber are depicted. VQIVYK (SEQ ID NO: 2) and orange-G are shown as sticks with non-carbon atoms colored by atom type. The carbons of VQIVYK (SEQ ID NO: 2) are colored white for one steric zipper and blue for the other. Two orange-G molecules (orange carbons) mediate contacts between two pairs of steric zippers; that is, orange-G is located between the protofilaments composing the fiber. In panel A, the view looks down the fiber axis. In panel B, the view is perpendicular to the fiber axis. Only the two sheets that are in contact with orange-G are shown. Backbone atoms are not shown. The unit cell dimension of the crystal along the fiber axis (4.83 Å) is indicated. The length of orange-G spans multiple unit cells of the fibril; that is, the dimensions of the small molecule and the fibril unit cell are incommensurate (see Example II). Panel C is an inset of panel B, focusing on the network of salt links (pink lines) between the sulfonic acid groups of two orange-G molecules and six lysine residues and with zinc cations (brown spheres). (D) Micro-crystals of VQIVYK (SEQ ID NO: 2) co-crystallized with orange-G.

FIG. 5 shows binding cavities of orange-G within fibers of the VQIVYK (SEQ ID NO: 2) segment from the tau protein. Orange-G is bound between steric zippers of VQIVYK (SEQ ID NO: 2), i.e., internally to a bundle of protofilaments. The VQIVYK (SEQ ID NO: 2) segment is located at the third repeat of the tau protein. Since there are many isoforms of tau, we will number the VQIVYK (SEQ ID NO: 2) residues 1-6 for simplicity. Peptide segments, forming β-sheet structures, are shown as arrows and sticks, colored by atom type with carbons in white. Orange-G carbons are in orange. Surface is shown for peptide atoms contacting the orange-G molecule (shown as spheres). The view in (A) looks down the fiber axis. The view in (B) is perpendicular to the fiber axis. Only side chains of interacting residues are shown. The solvent-accessible surface area of the fiber buried by orange-G (Methods) is 309 Å² and about 40% hydrophobic (contributed by the side chains of Val4 and the carbon chain of Lys6) and 60% polar (contributed by Gln2, Lys6, and the C-terminus). In (B), only one orange-G molecule and the β-sheets directly contacting it are shown. Zinc atoms are shown as brown spheres. It is noteworthy that interactions between lysine residues, zinc cations, and negatively charged groups are a motif observed in the Protein Data Bank; for example see [72].

FIG. 6 shows models of DDNP and curcumin bound to the VQIVYK (SEQ ID NO: 2) fiber based on incompletely differentiated electron density. Panels A and D are micro-crystals of VQIVYK (SEQ ID NO: 2) co-crystallized with DDNP and curcumin, respectively. In the structure of the complexes with DDNP (B-C) and curcumin (E-F), VQIVYK (SEQ ID NO: 2) is packed in a form having a steric zipper with one β-sheet shifted in relation to the other β-sheet (cartoon arrows). The carbons of VQIVYK (SEQ ID NO: 2) are colored white for one steric zipper and blue for the other. VQIVYK (SEQ ID NO: 2), DDNP, and curcumin are shown as sticks with non-carbon atoms colored by atom type. Six layers of the fiber are depicted. In panels B and E, the view looks down the fiber axis. In panels C and F, the view is perpendicular to the fiber axis. In both complexes, only the VQIVYK (SEQ ID NO: 2) segment is modelled into the electron density, and in both, there is apparent Fo-Fc difference electron density (shown as mesh, +3σ in green and −3σ in red) located in the void formed by the shift of the steric zipper. The positive density (part of the structure that has not been modelled, green mesh) displays a continuous tube-like shape, running along the fiber axis. We attribute this density to the presence of the small molecules, yet it is insufficiently undifferentiated to manually fit atoms into it in detail. DDNP (B-C, two molecules are shown) and curcumin (E-F) (both in magenta carbons) have been computationally docked (See Example IA) into the structures and fit reasonably well into the positive density. The unit cell dimension of the crystal along the fiber axis is indicated. The length of both DDNP and curcumin spans multiple unit cells of the fibril; that is, the dimensions of the small molecule and the fiber unit cell are incommensurate.

FIG. 7 shows binding cavities of DDNP or curcumin within fibers of the VQIVYK (SEQ ID NO: 2) segment from the tau protein. VQIVYK (SEQ ID NO: 2) segments, forming parallel β-sheet structures, are shows as arrows and sticks, colored by atom type with carbons in white. Docked DDNP (A and B) and curcumin (C and D) are shown as spheres with carbons colored magenta. Both small molecules are bound in the void formed within two shifted steric zippers. Surface rendering is shown for peptide atoms contacting the small molecules. The solvent-accessible surface area of the fiber buried by DDNP or curcumin is 242 Å² or 351 Å², respectively, and is about 50% hydrophobic (contributed by the side chain of Val1 and Ile3) and 50% polar (contributed by the hydroxyl of Tyr5 and the N-termini). The view in panels A and C looks down the β-sheets (fiber axis). The view in panels B and D is perpendicular to the fiber axis, with β-strands running horizontally.

FIG. 8 shows the chemical structures of the small molecule binders used for co-crystallization.

FIG. 9 shows that the crystal structure of the KLVFFA (SEQ ID NO: 1) segment from Aβ complexed with orange-G enjoy extensive interactions between two orange-G molecules and the fiber. The KLVFFA (SEQ ID NO: 1) segments are packed as pairs of O-sheets with orange-G bound internally to the steric zipper. The asymmetric unit of the crystal contains four peptide segments, two orange-G molecules, and 11 water molecules. Here, four layers of β-strands and two steric zippers are shown. KLVFFA (SEQ ID NO: 1) and orange-G are shown as sticks with non-carbon atoms colored by atom type. The anti-parallel strands (cartoon arrows) are alternately colored white and blue, with the adjacent steric zipper colored in darker hues. The two orange-G molecules in the asymmetric unit, with carbons in orange and brown, display similar interactions with the fiber. In panel A, the view looks down the fiber axis. In panels B-C, the view is perpendicular to the fiber axis. The sulfonic acid groups of orange-G form salt links (pink lines) with five lysine residues, four protruding from facing β-sheets of the steric zipper and one from the adjacent zipper (only the latter is presented as pink lines in panel A). Only side chains of residues participating in salt links are shown. One of the sheets from the adjacent pair and one orange-G molecule are removed for clarity.

FIG. 10 shows crystals of the KLVFFA (SEQ ID NO: 1) segment from Aβ and of the VQIVYK (SEQ ID NO: 2) segment from the tau protein grown with and without orange-G. (A-B) Micro-crystals of the KLVFFA (SEQ ID NO: 1) segment of Aβ grown under identical conditions (See Example IA) with (A) and without (B) orange-G. (C-D) Micro-crystals of the VQIVYK (SEQ ID NO: 2) segment of the tau protein grown under identical conditions (See Example IA) with (C) and without (D) orange-G.

FIG. 11 shows crystal structures used as controls for the complexes of the VQIVYK (SEQ ID NO: 2) segment from the tau protein with DDNP and curcumin. (A) Micro-crystals of VQIVYK (SEQ ID NO: 2) co-crystallized with DDNP; the structure is shown in panel C. (B) Micro-crystals of VQIVYK (SEQ ID NO: 2) crystallized under identical conditions to the crystals in panel A, lacking DDNP (See Example IA). The structure is shown in panel E. (D) The structure of VQIVYK (SEQ ID NO: 2) co-crystallized with DDNP, grown under different crystallization conditions than the structure shown in panel C (See Example IA). (F) Micro-crystals of VQIVYK (SEQ ID NO: 2) co-crystallized with curcumin; the structure is shown in panel G. (I) Micro-crystals of VQIVYK (SEQ ID NO: 2) crystallized under identical conditions to the crystals in panel F, lacking curcumin (See Example IA). The structure is shown in panel. H. In panels C-E and G-H, six layers of the VQIVYK (SEQ ID NO: 2) fiber are depicted. The VQIVYK (SEQ ID NO: 2) segment pack in parallel β-sheets (represented as cartoon arrows with white carbons). The view is perpendicular to the fiber axis, with β-strands running horizontally. Only the VQIVYK (SEQ ID NO: 2) segment was modelled into the electron density. The difference electron density Fo-Fc map is shown as mesh (+3σ in green and −3σ in red), indicating missing atoms in the model. The crystals grown without the small molecule, either DDNP or curcumin (panels B and I, respectively), are colorless, whereas the co-crystals are colored (panels A and F, respectively). Moreover, the VQIVYK (SEQ ID NO: 2)ₐₚₒ structures also lack the positive density (part of the structure that has not been modelled, green mesh) that we attribute to the presence of the small molecule (panels E and versus panels C and G, respectively). Both structures of VQIVYK (SEQ ID NO: 2) complexed with DDNP, grown under different crystallization conditions (panels C and D), show a similar, tube-like, positive electron density map, supporting the attribution of DDNP to this density.

FIG. 12 shows electron density maps and simulated annealing composite omit maps of the KLVFFA (SEQ ID NO: 1) segment from Aβ complexed with orange-G. The KLVFFA (SEQ ID NO: 1) segments and orange-G molecules are shown as sticks with non-carbon atoms colored by atom type. The β-sheets are formed via stacks of anti-parallel strands, alternately colored with carbons in white and in blue. The carbons of the orange-G molecules are colored orange. Water molecules are shown as aqua spheres. The view here is perpendicular to the fiber axis. (A-C) The electron density 2Fo-Fc map is shown as grey mesh (1.3σ). The difference electron density Fo-Fc map is shown as mesh (+3σ in green and −3σ in red). (D-F) The simulated annealing composite omit 2Fo-Fc map (10% omitted) is shown as grey mesh (1.3σ). Panels B-C and D-E focus on the two orange-G molecules in the asymmetric unit.

FIG. 13 shows an electron density map of the VQIVYK (SEQ ID NO: 2) segment from the tau protein complexed with orange-G. The VQIVYK (SEQ ID NO: 2) segment and orange-G are shown as sticks with carbon atoms colored grey and orange, respectively, and non-carbon atoms colored by atom type. The view in panel A looks down the fiber axis. The view in panel B is perpendicular to the fiber axis and focuses on orange-G. The electron density 2Fo-Fc map is shown as grey mesh (1.3σ). The difference electron density Fo-Fc map is shown as mesh (+3σ in green and −3σ in red).

FIG. 14 shows a flow chart of experiments to identify additional small molecule binders/inhibitors. The sequence KLVFFA (SEQ ID NO: 1) is indicated as an example of an amyloid-like fibril.

FIG. 15 shows another flow chart of experiments to identify additional small molecule binders/inhibitors.

FIG. 16 shows a cartoon of a proposed model of the mechanism by which fiber-binding molecules can alter amyloid aggregation. Left panel, the equilibrium of monomer, oligomer and fibril without fibril-binding compounds; Right panel, when a fibril-binding compound is added, the compound (green) binds to the side of the fibril, which stabilizes the fibril and thus shifts the equilibrium from monomer/oligomer to fibril.

FIG. 17 shows the reduction of Aβ toxicity, as measured in MIT assays, and the lack of reduction of Aβ fiber formation by compounds of the invention. A. Nine (9) compounds reduce Aβ toxicity to mammalian cell lines (PC12 cells in orange color; Hela cell in green). The results are shown for 2 to 4 independent experimental replicates (4 replicates per sample per concentration for each experiment). B. The representative compounds (BAF31, BAF26 and BAF11) reduce Aβ cyto-toxicity in a dose dependent manner. C. EM image of Aβ alone right before adding to the cell medium. D-L. The EM images of Aβ with the BAF compounds (BAF1, BAF4, BAF8, BAF11, BAF12, BAF14, BAF26, BAF30 and BAF31) right before adding to the cell medium. The compounds that inhibit Aβ toxicity do not inhibit Aβ fibrillation. The bar in each panel indicates 200 nm. Without being bound by any particular mechanism, this result suggests that the compounds may reduce toxicity by shifting oligomers into the fiber state.

FIG. 18 shows the chemical structures of seven active compounds of the invention.

FIG. 19 shows toxicity studies of compound BAF11 and some active derivatives thereof.

FIG. 20 shows toxicity studies of compound BAF30 and an active derivative thereof.

FIG. 21 shows a refined model of an amyloid pharmacophore, based on the overlay of structural models of the active compounds.

FIG. 22 shows the geometries defined in the amyloid pharmacophore shown in FIG. 21. The carbonyl group is used to represent the H-bond acceptor (or negative charge) of the inhibitor, and the naphthalene ring is used to represent the planar aromatic portion of the inhibitor. The defined interactions and geometries are detailed in Example III.

### DESCRIPTION

This application relates, e.g., to computer-based methods for designing and/or selecting (screening for) small molecule compounds which bind to amyloid fibers and/or which inhibit a biological function of the amyloid (e.g, inhibit amyloid-mediated cellular toxicity). The present inventors discovered that by co-crystallizing fiber-forming segments of amyloid proteins (e.g. Aβ and tau) with small molecule binders and by determining the structures of the resulting microcrystals by X-ray microcrystallography, they were able to characterize features of the drug binding environment (e.g. binding surfaces and/or binding pockets) of the amyloid binders, which allow for computer-based identification of additional small molecules that exhibit improved binding and/or inhibitory properties compared to the small molecules used to generate the co-crystals.

This represents the first time that, by using the adhesive segments of amyloid-forming proteins (such as Aβ), which on their own, isolated from the rest of the protein, form amyloid-like fibers, and growing co-crystals of such segments complexed with amyloid-binding ligands (to form microcrystals of about 1 micrometer in cross section), recording useful diffraction data from them, and determining the structures, it was possible to perform structure-based computational design of improved small molecule diagnostic and therapeutic agents. An improved docking program is also disclosed, which allows one to apply a docking program to identify compounds by targeting the amyloid fibril structure, and to successfully identify active compounds by docking a large compound database. (about 18,000 compounds) to amyloid fibril structures.

Compounds identified by methods of the invention, pharmaceutical compositions comprising the compounds, methods of using the compounds for diagnosis and/or treatment of amyloid-mediated diseases or conditions, and computer-related embodiments, such as a computer-readable medium providing the structural representation of a co-crystal of a protofilament of an amyloid protein with a small molecule that is known to bind to the amyloid protein, are also described.

An advantage of the small molecule compounds of the invention is that they are expected to readily cross the blood brain barrier. This property enhances their ability to visualize, for example, amyloid plaques and/or tau tangles in the brains of subjects having amyloid-mediated diseases, and to be effective as therapeutic agents for the treatment of such subjects.

One aspect of the invention is a method for determining on a computer the relevant criteria for designing or selecting (screening for) on a computer a small molecule amyloid binder or inhibitor (e.g., for creating a computer-based replica or a pharmacophore representing the criteria), comprising
a) co-crystallizing a protofilament of an amyloid protein with a small molecule that is known to bind to the amyloid protein (to form microcrystals); and
b) determining on a computer the three-dimensional structure of the co-crystal, thereby determining the atomic coordinates of the binding surface or binding pocket. The determining step on the computer comprises recording diffraction data from the co-crystals (which amyloid-like fibers invariably form).
   The method may further comprise
c) docking test compounds to the crystal structure determined in b) on a computer, and
d) selecting test compounds which exhibit a calculated binding energy below that of the small molecule used to form the co-crystal made in a), as candidate amyloid binders.

In one embodiment of the invention, test compounds are selected which exhibit an energy below an empirically determined threshold value based on the comparative values of energy found for co-crystals made with many candidate amyloid binders. The threshold values will differ depending on which co-crystal is being analyzed and which docking program used in the analysis. For example, when using the energy score obtained with the ROSETTA program, the calculated binding energy of Orange-G/Aβ co-crystals is about −8 kcal/mol, so compounds with an energy of below −8 kcal/mol are selected. For other co-crystals, using the ROSETTA energy score, the energy values can range from about −5 kcal/mol to about −15 kcal/mol. When using other programs, such as AutoDock or DOCK, the energy values may be considerably different. In embodiments of the invention, a structural representation of the co-crystal is provided in a storage medium on a computer; and a computer is used to apply structure-based drug design techniques to the structural representation.

In one embodiment of this method, the amyloid protein is Aβ, and the small molecule is a polar (e.g., charged) molecule comprising one or more flat aromatic rings (e.g., a polar molecule), such as Orange-G. The atomic coordinates of the three-dimensional structure are shown in Table 3, and the amino acid residues of the amyloid molecule which contact the amyloid binder are selected from one or more of Lys16, Leu17, Val18, Phe19, or Phe20, or combinations thereof (e.g., the binding site or pocket comprises one or more of these amino acid residues).

In another embodiment, the amyloid protein is tau, and the small molecule is a polar (e.g. charged) molecule comprising one or more flat aromatic rings (e.g., a polar molecule), such as Orange-G. The atomic coordinates of the three-dimensional structure are shown in Table 4, and the amino acid residues of the amyloid molecule which contact the amyloid binder are selected from one or more of Gln2, Val4, or Lys6, or combinations thereof.

In another embodiment, the amyloid protein is tau, and the small molecule is an elongated (having a ratio of length to width of greater than 2:1) apolar molecule, such as curcumin or DDNP. The atomic coordinates of the three-dimensional structure are shown in Table 5 or 6, respectively, and the amino acid residues of the amyloid molecule which contact the amyloid binder are selected from one or more of Val1, Gln2, Ile3, Val4, Tyr5 or Lys6, or combinations thereof.

Another aspect of the invention is a method for designing or selecting (screening for) on a computer a candidate small molecule amyloid binder or inhibitor, comprising
a) docking test compounds to the binding site or binding surface determined from the three-dimensional structure of a co-crystal of a protofilament of an amyloid protein bound to a small molecule which is known to bind to the amyloid protein. In embodiments of this method, the atomic coordinates of the binding site or binding surface are as set forth in Tables 3-6 as indicated below, and amino acid residues of the amyloid molecule which contacts the amyloid binder are as indicated (e.g., the binding site or pocket comprises one or more of the amino acid residues as indicated):
   (i) Table 3 (based on an Orange-G/Aβ co-crystal), wherein the amino acid residues of the amyloid molecule are selected from one or more of Lys16, Leu17, Val18, Phe19 and Phe20, or combinations thereof; or
   (ii) Table 4, (based on an Orange-G/tau co-crystal), wherein the amino acid residues of the amyloid molecule are selected from one or more of Gln2, Val4 and Lys6, or combinations thereof; or
   (iii) Table 5 (based on a co-crystal of tau with curcumin), wherein the amino acid residues of the amyloid molecule are selected from one or more of Vail, Gln2, Ile3, Val4, Tyr5 and Lys6, or combinations thereof;
   (iv) Table 6 (based on a co-crystal of tau with DDNP), wherein the amino acid residues of the amyloid molecule are selected from one or more of Val1, Gln2, Ile3, Val4, Tyr5 and Lys6, or combinations thereof; and
(b) selecting test compounds which exhibit an energy below that of the small molecule used to form the co-crystal made in a), as candidate amyloid binders

In one embodiment of the invention, the docking in a method as above is accomplished by a docking program in which the test molecule and protein side chain tortion angles and small molecule rotamers are sampled in a near native perturbation fashion. Many of the currently available docking programs are high resolution and are designed to fit test molecules into deep binding pockets of whole proteins. For the 3-D structures of the present invention, in which the binding surfaces or binding pockets are much shallower, it is desirable to use a docking program at lower resolution, allowing for more rapid screening. In many currently available docking programs, all possible side chain angles and revolutions are tested. For docking test molecules to the present 3-D structures, it is desirable to sample ligand and protein side-chain torsion angles and ligand rotamers in a near “native” perturbation fashion. By near “native” is meant limiting the possible side chain torsion angles to deviations (+/−0.33, 0.67, 1 sd) around each input torsion, based on the standard deviation value of the same torsion bin from the backbone-dependent Dunbrack rotamer library. See Example III for details.

Any of the preceding methods can further comprise (a) testing the candidate amyloid binders for their ability to inhibit amyloid-mediated cell toxicity, and identifying and selecting candidate amyloid inhibitors which inhibit amyloid-mediated cell toxicity to a greater degree than the small molecule which was co-crystallized with the amyloid; (b) characterizing and validating the candidate binders by X-ray crystallography, NMR spectroscopy (titration), ITC (isothermal titration calorimetry), thermal denaturation, mass spectrography, SPR (surface plasmon resonance), to measure the binding affinity to the amyloid fibers and also to oligomers, and/or an activity assay; (c) deriving on a computer a refined pharmacophore based on the identified candidate amyloid inhibitors (e.g. using methods as discussed herein).

Starting with the refined pharmacophore derived above, one can test a new set of candidate amyloid binders by repeating the docking and selecting steps, and testing the candidate amyloid binders for their ability to inhibit amyloid-mediated cell toxicity, in order to identify a further refined pharmacophore. Then, starting with this further refined pharmacophore, one can repeat the docking and screening steps, and test the candidate amyloid binders for their ability to inhibit amyloid-mediated cell toxicity in order to identify a yet further refined pharmacophore. This series of steps can be reiterated (repeated) as many times as desired.

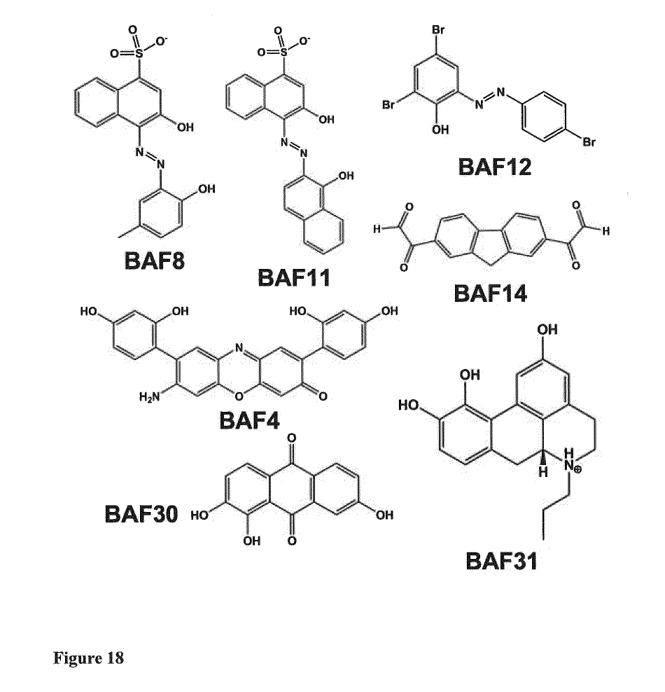

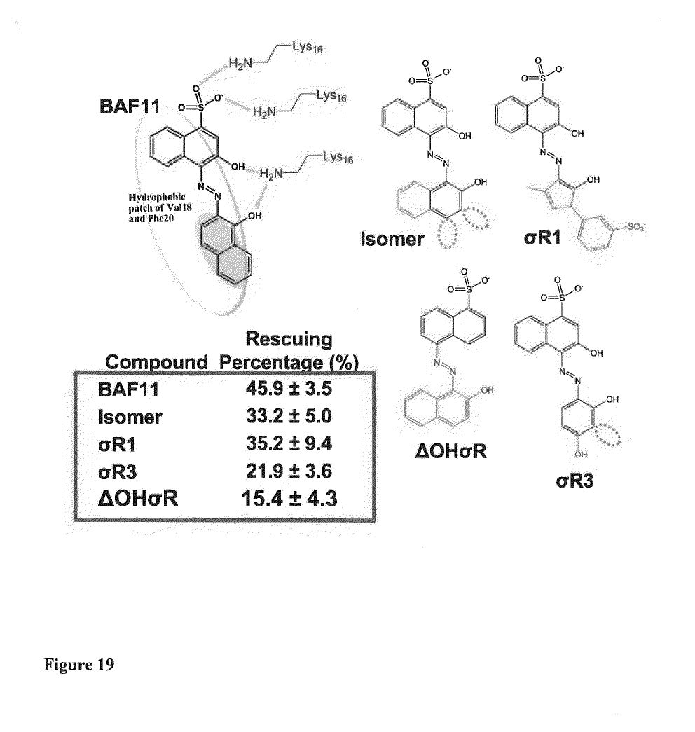

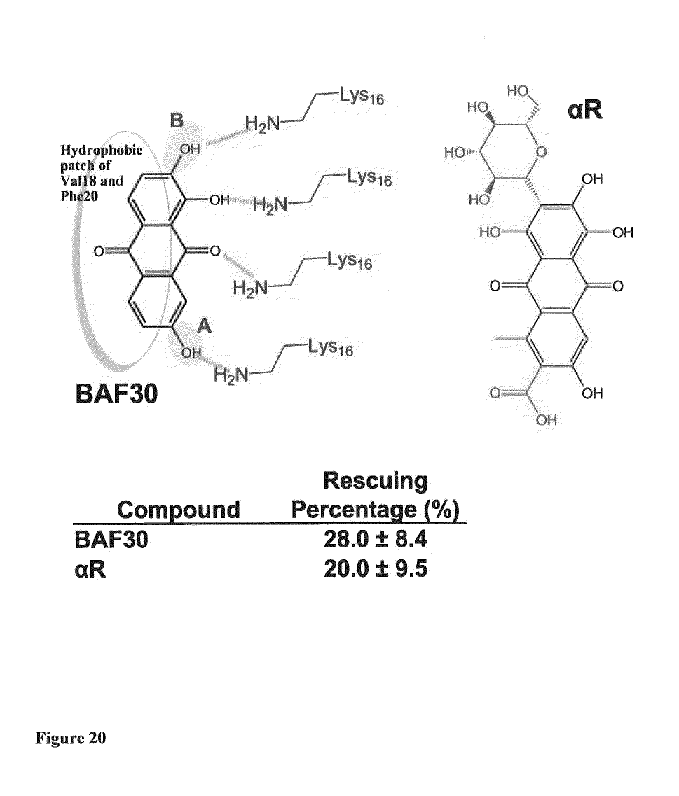

Another aspect of the invention is a pharmaceutical composition comprising one or more of the compounds BAF4, BAF8, BAF11, BAF12, BAF14, BAF30 or BAF31, as shown in FIG. 18, or the derivatives of BAF11—the isomer, σR1, σR3 or ΔOHσR—as shown in FIG. 19, or the derivative of BAF30-αR1—as shown in FIG. 20, or a pharmaceutically acceptable salt, hydrate, solvate or metal chelate thereof, and a pharmaceutically acceptable carrier. These compounds are sometimes referred to herein as the first set of (twelve) active compounds of the invention. The pharmaceutical compositions may be detectably labeled, e.g. with a radioactive or fluorescent label, or with a label that is suitable for detection by positron emission spectroscopy (PET).

Another aspect of the invention is a method for determining the presence of Aβ or tau oligomers or fibers (particularly fibers) in a sample, comprising contacting a sample suspected of comprising such oligomers or fibers with an effective amount of one or more of the 35 BAF compounds listed in Table 9, or suitable derivatives thereof. In one embodiment, the compounds are selected from one or more of the first set of (twelve) active compounds of the invention. The compounds may be detectably labeled. The contacting step is followed by measuring the amount of (bound) label in the sample, wherein a statistically significantly higher amount of label than that in a control sample lacking fibers indicates the presence of the fibers in the sample. In embodiments of this method, the determination is carried out on an in vitro sample (e.g. a tissue culture sample) or is carried out on a subject (e.g. the sample is removed from the subject, and can be, for example, blood or cerebral spinal fluid (CSF)). When the determination of Aβ or tau oligomers or fibers is in a sample from a subject, the method can be a method for diagnosing the presence of an amyloid-mediated disease or condition, such as Alzheimer's disease. Compounds that are found by a method of the invention to diagnose one disease or condition may also be useful for diagnosing a different amyloid-mediated disease or condition; and compounds found to reduce amyloid-mediated toxicity or to be useful for treating one amyloid-mediated disease or condition may also be useful for reducing amyloid-mediated toxicity or for treating a different amyloid-mediated disease or condition.

Compounds of the invention can also be used to detect the presence of Aβ or tau oligomers or fibers in a subject, in vivo, comprising introducing into the subject an effective amount of one or more of the 35 BAF compounds listed in Table 9, or suitable derivatives thereof. In one embodiment, the compounds are selected from one or more of the first set of (twelve) active compounds of the invention. In this method, the compound is labeled with a nuclide that can be detected by PET. The amount of bound label in the brain is them measured by PET (imaging the brain by PET). A statistically significantly higher signal than that in a control sample lacking the oligomers or fibers indicates the presence of the oligomers or fibrils in the brain of the subject.

Another aspect of the invention is a method for reducing or inhibiting amyloid-based (cellular) toxicity, comprising contacting amyloid protofilaments with an effective amount of one or more of the first set of (twelve) active compounds of the invention. This method can be carried out in vitro (e.g. in tissue culture) or in vivo (in a subject).

When carried out in a subject, the method can be a method for treating an amyloid-mediated disease or condition (e.g. a disease or condition mediated by Aβ or tau), comprising administering to a subject having or likely to have the disease or condition an effective amount of one or more of the set of twelve amyloid-inhibiting compounds. In one embodiment of the invention, a cocktail of more than one of these amyloid-inhibiting compounds is administered. As is described elsewhere herein, the inventors observed that different amyloid polymorphs bind different small molecules, suggesting that a cocktail of compounds directed against more than one of the polymorphs may provide improved therapies by binding to the several amyloid polymorphs present.

Another aspect of the invention is a computer readable medium providing the structural representation of a co-crystal of a protofilament of an amyloid protein with a small molecule that is known to bind to the amyloid protein.

Another aspect of the invention is a kit for carrying out any of the methods described herein (e.g., for identifying new compounds which bind to amyloid and/or inhibit amyloid toxicity, for diagnostic assays, for therapeutic applications, etc).

In the Examples shown herein, the present inventors first used a core fiber-forming hexamer segment from Aβ [KLVFFA (SEQ ID NO:1)] and one from tau [VQIVYK (SEQ ID NO:2)] to form co-crystals with low molecular weight compounds that were reported to bind to and/or to inhibit fibrillation of the amyloid fibers—the dye orange-G, the natural compound curcumin, and the Alzheimer/s diagnostic compound DDNP; and they then determined the atomic structures of the fiber-like complexes by X-ray microcrystallography. The atomic coordinates of the crystal structures of Orange-G/Aβ, Orange-G/tau, curcumin/tau and DDNP/tau are shown in Tables 3-6, respectively. The first two crystal structures are deposited in the Protein Data Bank (PDB) with accession codes 3OVJ and 3OVL. The rest of the structures and crystallographic tables are accessible at the world wide web site people.mcbi.ucla.edu/meytal/CoCrystalPaper.

The atomic structures of the fiber-like complexes reveal that they consist of pairs of β-sheets, with small molecules binding between the sheets, roughly parallel to the fiber axis. Cylindrical cavities run along the β-spines of the fibers. Negatively charged orange-G wedges into a specific binding site between two sheets of the fiber, combining apolar binding with electrostatic interactions with lysine side chains of adjacent sheets, whereas uncharged compounds slide along the cavity. The three dimensional (3-D) structures thus determined allow for a structure-based design of improved small molecule diagnostics and therapeutics. The structural characteristics which allow for such design are sometimes referred to herein as pharmacophores.

Having obtained the co-crystals and the 3-D structures, the inventors developed a computer-based method to identify new candidates for small molecule amyloid binders. As proof of principle, the inventors employed a 3-D structure determined from a co-crystal of the Aβ fiber-forming segment, KLVFFA (SEQ ID NO: 1), and the negatively charged small molecule, Orange-G. They first assembled a database of test compounds containing a total of about 20,000 small molecules, which met certain initial criteria as described in Example III. The test molecules were docked on the computer to the crystal structure to determine if they fit, and to determine the energy of the fit. By determining for each test molecule the position and orientation having minimal energy, and using a threshold cut-off value that is below the calculated binding energy of the Orange-G molecule in the co-crystal (e.g., when using the Rosetta program exemplified herein, about 8 kcal/mol), 35 candidate amyloid binding molecules were identified for further study. See Table 9.

These 35 candidate molecules were then further characterized and validated by other criteria, including NMR titration, electron microscopy, and cell viability studies. Nine compounds were shown to inhibit amyloid cellular toxicity to a greater degree than the Orange-G used to form the original co-crystals. Of these, 7 compounds have not, to our knowledge, been reported to reduce amyloid toxicity and are particularly good candidates for therapeutic and/or diagnostic agents for amyloid diseases.

In subsequent steps, the inventors expanded the set of test compounds to include derivatives (homologs) of the active molecules described above. 25 such derivatives were selected, based on the crystal structure described above. Viability assays revealed that 7 of these derivatives can reduce amyloid toxicity, 5 of which have not, to our knowledge, been reported to inhibit amyloid toxicity, giving a total of 12 new small molecule amyloid inhibitors.

Using the identified amyloid inhibitors, the inventors designed a more refined general set of rules for identifying compounds which bind to Aβ fibers (a more refined pharmacophore), which can then be used, e.g. in a method as described above, to identify additional and/or improved amyloid inhibitors. This process can be reiterated for as many rounds as desired, to obtain additional, improved agents for use as diagnostic or therapeutic agents.

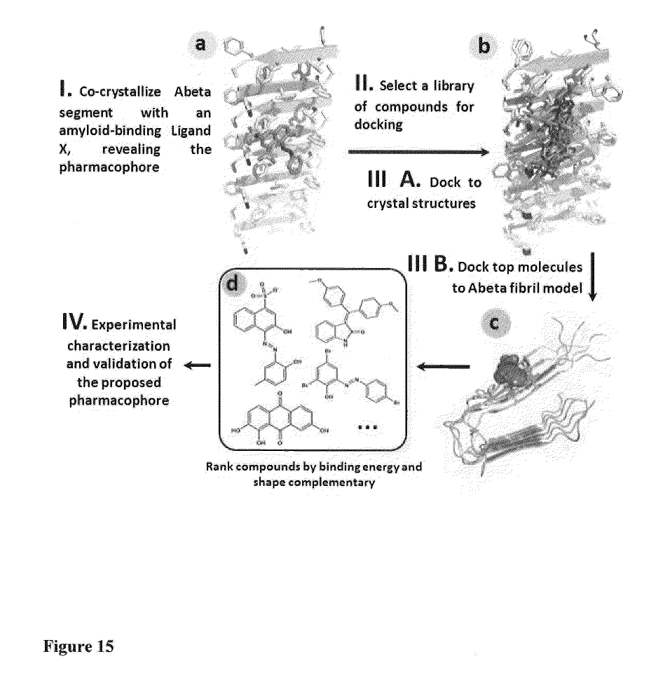

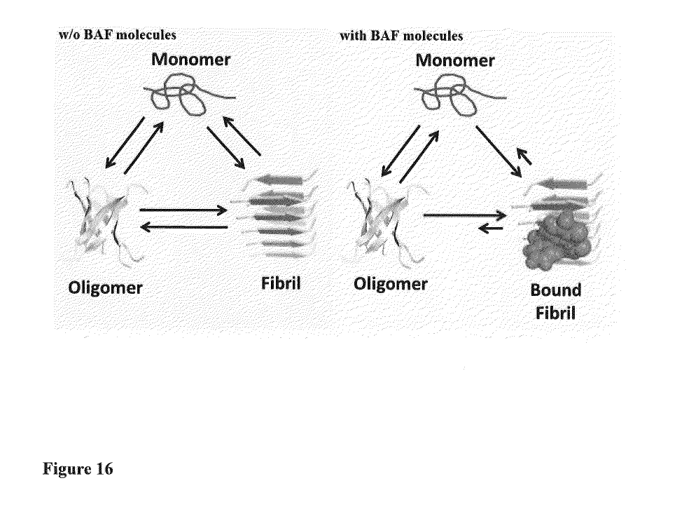

Flow charts shown in FIGS. 15 and 16 summarize the studies discussed above.

As used herein, the term pharmacophore” refers to a specific, three-dimensional map of chemical and biological structures, properties, and features common to a set of ligands that exhibit a particular activity. A pharmacophore can be used as a model for the design of specific molecules that exhibit the same structural and functional features as the ligand(s) from which the pharmacophore was derived. Examples of pharmacophores according to the invention are displayed throughout this application.

Features of pharmacophores that relate to functional, structural, chemical or biological descriptors that describe a substituent and interaction of ligands with their receptors or binding sites include, e.g, hydrogen bond donors, hydrogen bond acceptors, hydrophobic regions, hydrophilic regions, ionizable regions, or aromatic rings. The features may further be described by the distances separating the features. For example, a feature may be a hydrogen bond donor that is 3 Å from a hydrogen bond acceptor. Pharmacophore features may be arranged in three-dimensional space and define points of interaction with the residues lining a binding site. In addition, features may further be described by torsional degrees of freedom of an atom or groups of atoms that define distinct, low energy conformations.

As used herein, the following terms have the meanings as indicated:

The term “small molecule” refers to a low molecular weight organic compound, e.g. having a molecular weight of less than about 800 Daltons (e.g. <700, 600, 500, 400, 300 Daltons). Small peptides (e.g. about 6 amino acids) are not included. As used herein, “about” means plus or minus 5% of the value.

An “amyloid” protein refers to one of a class of proteins having the structural and functional characteristics described in the Background Information section of this application and in the references cited therein. Inappropriately folded (misfolded) versions of the proteins interact with one another or other cell components to form insoluble fibrils (e.g. plaques or tangles). A skilled worker will recognize a wide variety of amyloid proteins that can be used in a method of the invention to design or select small molecule binders or inhibitors. These amyloid proteins have been implicated in the etiology of a variety of diseases or conditions, including neurodegenerative ones, and include, e.g., beta amyloid (Alzheimer's disease, cerebral amyloid angiopathy), tau (Alzheimer's disease and a large number of tauopathies, including frontotemporal dementia and progressive supranuclear palsy), amylin (diabetes type 2), PrP (Creutzfeldt-Jacob Disease, fatal familial insomnia, other prior-based conditions), SOD1, TDP-43, FUS (ALS), alpha-synuclein (Parkinson's disease), p53 (many cancers), and beta 2 microglobulin (dialysis related amyloidosis).

Such conditions are sometimes referred to herein as “amyloid-mediated” conditions or diseases. A disease or condition that is “mediated” by an amyloid is one in which the amyloid plays a biological role. The role may be direct or indirect, and may be necessary and/or sufficient for the manifestation of the symptoms of the disease or condition. It need not necessarily be the proximal cause of the disease or condition

A “protofilament” refers to the basic unit of a mature amyloid fiber. For the Abeta and tau structures described herein, each protofilament generally contains two of the beta sheets. Each sheet is formed from stacks of identical fiber-forming segments, as represented by the hexamers described herein. A pair of sheets forms a “cross-β spine” of the protofilament.

An “oligomer” amyloid structure contains between about 20 and 1,000 amyloid molecules.

The terms amyloid “fiber” and “fibril” are used interchangeably herein, and refer to structures with thousands of amyloid molecules.

“Fibrillation” refers to the aggregation of amyloid molecules to form fibers.

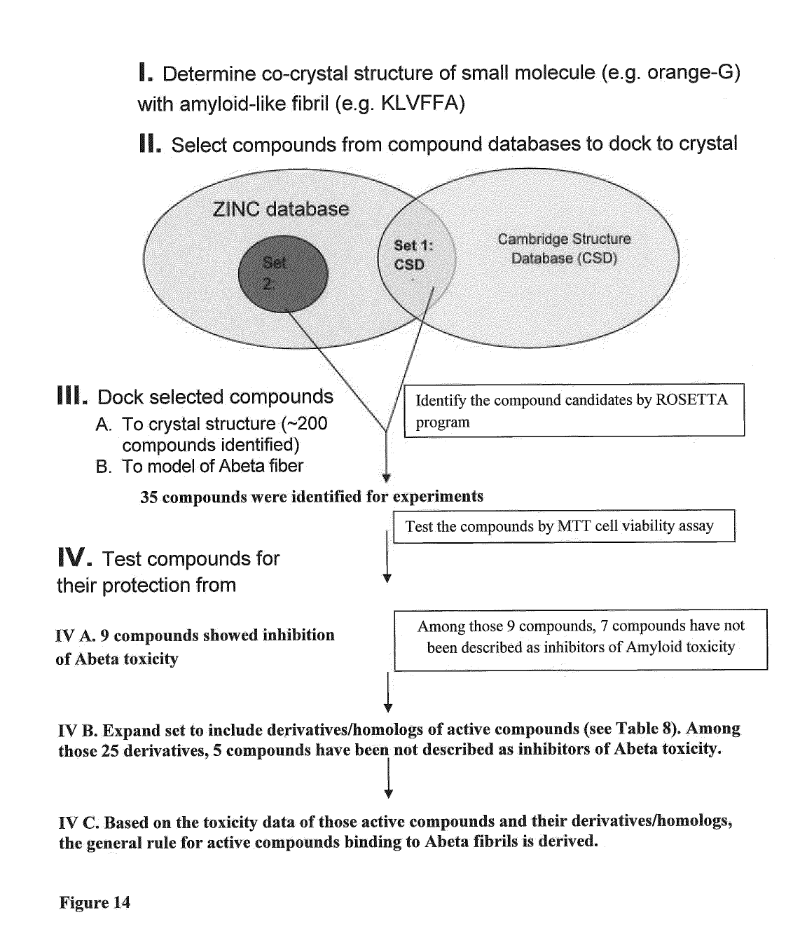

Without wishing to be bound by any particular mechanism, it is suggested, particularly with regard to Alzheimer's disease, that soluble aggregation intermediates such as amyloid oligomers are more toxic than amyloid fibers, while fibrils may serve as reservoirs of toxic oligomers. In this suggested model, fiber-binding molecules can inhibit amyloid toxicity by shifting the equilibrium from toxic oligomers toward end-stage fibers. See, e.g., FIG. 14 and Bieschke et al. Small-molecule conversion of toxic oligomers to nontoxic β-sheet-rich amyloid fibrils, Nature Chemical Biology 8, 93-101 (2012).

An “amyloid binder” is a molecule which binds to an amyloid, preferably to the degree required to detect the presence of the amyloid (e.g., in a diagnostic assay). In some, but not all, cases, an amyloid binder can also elicit a biological effect (such as the inhibition of amyloid-induced cellular toxicity), in which case it is referred to herein as an “amyloid inhibitor.”

Any of a variety of fiber-forming segments of amyloid proteins can be used to generate co-crystals with small molecule amyloid binders, in addition to the hexamers described herein. These include, e.g., for Abeta, NKGAII (two polymorphic crystal forms) (SEQ ID NO:23), GAIIGL (SEQ ID NO:24), AIIGLM (SEQ ID NO:25), MVGGVVIA (2 POLYMOPRHIC CRYSTAL FORMS) (SEQ ID NO:26); MVGGVV (2 FORMS) (SEQ ID NO:27), GGVVIA (SEQ ID NO:28); for tau (Alzheimer's disease), VQIINK (SEQ ID NO:29); for Alpha synuclein (Parkinson's disease), GVTTVA (SEQ ID NO:30), GVATVA (SEQ ID NO:31), VVTGVTA (SEQ ID NO:32), TGVTAVA (SEQ ID NO:33); for insulin (Injection amyloidosis, and keeping insulin from forming fibers while stored), VEALYL (SEQ ID NO:34), LYQLEN (SEQ ID NO:35); for lysozyme (lysozyme amyloidosis), IFQINS (SEQ ID NO:36), TFQINS (SEQ ID NO:37), for Islet amyloid polypeptide (aka IAPP or amylin)—Diabetes type 2, NNFGAIL (SEQ ID NO:38), SSTNVG (SEQ ID NO:39); for p53—Cancer, TITTLE (SEQ ID NO:40), LTITTLE (SEQ ID NO:41); for Beta-2-microglobulin—Dialysis amyloidosis, NHVTLS (SEQ ID NO:42), NHVTLSQ (SEQ ID NO:43), KDWSFY (SEQ ID NO:44); for Transthyretin—several different amyloidosis, TIAALLS (SEQ ID NO:45), AADTWE (SEQ ID NO:46), YTIAAL (SEQ ID NO:47), SOD1 (SEQ ID NO:48), GVIGIAQ (SEQ ID NO:49), GVTGIAQ (SEQ ID NO:50), DSVISLS (SEQ ID NO:51), VQGIINFE (SEQ ID NO:52), for Prion protein (aka PrP)—prion diseases CJD etc., GTHSQW (SEQ ID NO:53), GTHSQWN (SEQ ID NO:54), AGAAAA (SEQ ID NO:55), GAVVGG (SEQ ID NO:56), GYMLGS (SEQ ID NO:57), GYVLGS (SEQ ID NO:58), IIHFGS (SEQ ID NO:59), NQVYYR (SEQ ID NO:60), PMDEYS (SEQ ID NO:61), SNQNNF (SEQ ID NO:62), NQNNFV (SEQ ID NO:63); QHTVTT (SEQ ID NO:64).

Aspects of a method of the invention for designing and/or selecting candidate amyloid-binding compounds comprise determining on a computer the 3-D structure of the co-crystal, thereby determining the atomic coordinates of the binding pocket or binding surface (pharmacophore).

Techniques for determining the three-dimensional (3-D) structure of such a co-crystal are conventional and well-known in the art. See, e.g., the Examples herein. Such a determination can comprise providing a structural representation of the co-crystal in a storage medium on a computer.

The storage medium (computer readable medium) in which the co-crystal structural representation is provided may be, e.g., random-access memory (RAM), read-only memory (ROM e.g. CDROM), a diskette, magnetic storage media, hybrids of these categories, etc. The storage medium may be local to the computer, or may be remote (e.g. a networked storage medium, including the Internet). The present invention also provides methods of producing computer readable databases containing coordinates of 3-D co-crystal structures of the invention; computer readable media embedded with or containing information regarding the 3-D structure of a co-crystal of the invention; a computer programmed to carry out a method of the invention (e.g. for designing and/or selecting small molecule amyloid binders or inhibitors), and data carriers having a program saved thereon for carrying out a method as described herein.

Any suitable computer can be used in the present invention.

A “binding surface” or “binding pocket” refers to a site or region in a co-crystal of the invention that, because of its shape, likely associates with a substrate or ligand. Atomic coordinates of the co-crystals of the invention define the binding surface or pocket. The amino acid residues of the Aβ or tau hexamer segments used to form the co-crystals described herein, which bind to the ligands and where are therefore important for binding small molecules designed or selected by a method of the invention, include one of more of the following amino acid residues, or combinations thereof: for the Orange-G/Aβ co-crystals, Lys16, Leu17, Val18, Phe 19, and Phe20; for the Orange-G/tau co-crystals, Gln2, Val4, and Lys6; and for the DDNP or curcumin/tau co-crystals, Val1, Gln2, Ile3, Val4, Tyr5 or Lys6. The numbering of the amino acid residues is as described elsewhere herein.

In aspects of a method of the invention for designing and/or selecting candidate amyloid-binding compounds, test molecules (for small molecule amyloid binders) are “docked” in a computer to determine if they fit well and bind tightly. Docking aligns the 3-D structures of two or more molecules to predict the conformation of a complex formed from the molecules. According to the present invention, test molecules are docked with a co-crystal 3-D structure of the invention to assess their ability to interact with the amyloid. Docking can be accomplished by either geometric matching of the ligand and its receptor or by minimizing the energy of interaction. This generally requires rotation and translation of a compound to achieve the best alignment with the 3-D structure (pharmacophore), i.e., the lowest energy conformation or interaction.

Suitable docking algorithms are well-known to those of skill in the art and include, e.g., DOCK [Kuntz et al. (1982) J. Mol. Biol. 161:269-288; available from UCSF]; AUTODOCK [Goodsell & Olson (1990) Proteins: Structure, Function and Genetics 8:195-202; Available from Oxford Molecular (<http://www.oxmol.co.uk/>]; MOE-DOCK [Available from Chemical Computing Group Inc. (<http://www.chemcomp.com/>); FLExX [Available from Tripos Inc (<http://www.tripos.com)]; GOLD [Jones et al. (1997) J. Mol. Biol. 267:727-748]; and AFFINITY [Available from Molecular Simulations Inc (<http://www.msi.com/>)]. The docking method described in the Examples herein is a modified version of the RosettaLigand program.

The test compounds may be known compounds or based on known compounds. Suitable libraries of compounds will be evident to a skilled worker. Several such compound libraries are discussed in the Examples herein.

Alternatively, the test compounds may be designed and made de novo. The binding surface or pharmacophore of a co-crystal 3-D structure of the invention can be used to map favorable interaction positions for functional groups (e.g. protons, hydroxyl groups, amine groups, hydrophobic groups and/or divalent cations) or small molecule fragments. Compounds can then be designed de novo in which the relevant functional groups are located in the correct spatial relationship to interact with CD81.

Once functional groups or small molecule fragments which can interact with specific sites on the binding surface or in the binding pocket of a co-crystal of the invention have been identified, they can be linked in a single compound using either bridging fragments with the correct size and geometry or frameworks which can support the functional groups at favorable orientations, thereby providing a compound according to the invention. While linking of functional groups in this way can be done manually, perhaps with the help of software such as QUANTA or SYBYL, automated or semi-automated de novo design approaches are also available. These include, e.g., MCDLNG [Gehlhaar et al. (1995) J. Med. Chem. 38:466-72]; MCSS/HOOK [Caflish et al. (1993) J. Med. Chem. 36:2142-67; Eisen et al. (1994) Proteins: Str. Funct. Genet. 19:199-221]; LUDI [2 Bohm (1992) J. Comp. Aided Molec. Design 6:61-780); GROW [Moon & Howe (1991) Proteins: Str. Funct. Genet. 11:314-328]; GROUPBUILD [Rotstein et al. (1993) J. Med. Client. 36:1700]; CAVEAT Lauri & Bartlett (1994) Comp. Aided Mol. Design. 8:51-66]; RASSE [Lai (1996) J. Chem. Inf. Comput. Sci. 36:1187-1194]; and others.

An amyloid inhibitor of the invention inhibits a measurable amount of one or more functions of an amyloid (e.g. it can inhibit or reduce amyloid-mediated or induced cellular toxicity; disrupt the structure of an amyloid oligomer; bind to an amyloid oligomer or fiber; stabilize amyloid fibers, thereby shifting the equilibrium to favor the formation of fibers rather than oligomers; etc.) Methods for assaying such amyloid-mediated effects are conventional and well-known to those of skill in the art. Some such methods, e.g., for measuring amyloid-mediated cellular toxicity, are described in the Examples.

In aspects of the invention, candidate inhibitors are further characterized and/or validated by any of a variety of methods, including X-ray crystallography, NMR spectroscopy (titration), ITC (isothermal titration calorimetry), thermal denaturation, mass spectroscopy, SPR (surface plasmon resonance), to measure the binding affinity to the amyloid fibers and also to oligomers, and/or an activity assay. In one embodiment of the invention, amyloid-mediated cell toxicity is monitored by assaying for cell viability, using an assay such as the MIT assay. Such methods are conventional and well-known in the art; some of them are described in the Examples herein.

A compound of the invention can be in the form of a pharmaceutically acceptable salt, solvate or salt. Suitable acids and bases that are capable of forming salts with the compounds of the present invention are well known to those of skill in the art, and include inorganic and organic acids and bases. “Solvates” refers to solvent additions forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate. Hydrates are formed by the combination of one or more molecules of water with one of the substances in which the water retains its molecular state as H₂O, such combination being able to form one or more hydrate.

A “pharmaceutical composition” comprises a compound of the invention plus a pharmaceutically acceptable carrier or diluent. In some embodiments, the compound is present in an effective amount for the desired purpose.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use. For example, “pharmaceutically acceptable salts” of a compound means salts that are pharmaceutically acceptable, as defined herein, and that possess the desired pharmacological activity of the parent compound.

One aspect of the invention is a method for reducing or inhibiting amyloid-based cellular toxicity, or for treating an amyloid-mediated disease or condition, comprising contacting amyloid protofilaments with an effective amount of a compound of the invention, or, if the method is conducted in vivo (in a subject), administering an effective amount of the compound to the subject.

An “effective amount” of a compound or pharmaceutical composition of the invention is an amount that can elicit a measurable amount of a desired outcome, e.g. for a diagnostic assay, an amount that can detect a target of interest, such as an amyloid oligomer or fiber, or in a method of treatment, an amount that can reduce or ameliorate, by a measurable amount, a symptom of the disease or condition that is being treated.

A “subject” can be any subject (patient) in which amyloid molecules associated with an amyloid-mediated disease or condition can be detected, or in which the disease or condition can be treated by a compound of the invention. Typical subjects include vertebrates, such as mammals, including laboratory animals, dogs, cats, non-human primates and humans.

The compounds of the invention can be formulated as pharmaceutical compositions in a variety of forms adapted to the chosen route of administration, for example, orally, nasally, intraperitoneally, or parenterally, by intravenous, intramuscular, topical or subcutaneous routes, or by injection into tissue.

Suitable oral forms for administering the compounds include lozenges, troches, tablets, capsules, effervescent tablets, orally disintegrating tablets, floating tablets designed to increase gastric retention times, buccal patches, and sublingual tablets.

The compounds of the invention may be systemically administered, e.g., orally, in combination with a pharmaceutically acceptable vehicle such as an inert diluent or an assimilable edible carrier, or by inhalation or insufflation. They may be enclosed in coated or uncoated hard or soft shell gelatin capsules, may be compressed into tablets, or may be incorporated directly with the food of the patient's diet. For oral therapeutic administration, the compounds may be combined with one or more excipients and used in the form of ingestible tablets, buccal tablets, troches, capsules, elixirs, suspensions, syrups, wafers, and the like. For compositions suitable for administration to humans, the term “excipient” is meant to include, but is not limited to, those ingredients described in Remington: The Science and Practice of Pharmacy, Lippincott Williams & Wilkins, 21st ed. (2006) (hereinafter Remington's).

The compounds may be combined with a fine inert powdered carrier and inhaled by the subject or insufflated. Such compositions and preparations should contain at least 0.1% compounds. The percentage of the compositions and preparations may, of course, be varied and may conveniently be between about 2% to about 60% of the weight of a given unit dosage form.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. A syrup or elixir may contain the active compound, sucrose or fructose as a sweetening agent, methyl and propylparabens as preservatives, a dye and flavoring such as cherry or orange flavor.

Various other materials may be present as coatings or to otherwise modify the physical form of the solid unit dosage form. For instance, tablets, pills, or capsules may be coated with gelatin, wax, shellac or sugar and the like. Of course, any material used in preparing any unit dosage form should be pharmaceutically acceptable and substantially non-toxic in the amounts employed.

In addition, the compounds may be incorporated into sustained-release preparations and devices. For example, the compounds may be incorporated into time release capsules, time release tablets, and time release pills. In some embodiments, the composition is administered using a dosage form selected from the group consisting of effervescent tablets, orally disintegrating tablets, floating tablets designed to increase gastric retention times, buccal patches, and sublingual tablets.

The compounds may also be administered intravenously or intraperitoneally by infusion or injection. Solutions of the compounds can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the compounds which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants.

Sterile injectable solutions are prepared by incorporating the compounds in the required amount in the appropriate solvent with various of the other ingredients enumerated above, as required, followed by filter sterilization. In the case of sterile powders for the preparation of sterile injectable solutions, the preferred methods of preparation are vacuum drying and freeze drying techniques, which yield a powder of the active ingredient plus any additional desired ingredient present in the previously sterile-filtered solutions.

For topical administration, the compounds may be applied in pure form. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Other solid carriers include nontoxic polymeric nanoparticles or microparticles. Useful liquid carriers include water, alcohols or glycols or water/alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Useful dosages of the compounds of formula I can be determined by comparing their in vitro activity, and in vivo activity in animal models. Methods for the extrapolation of effective dosages in mice, and other animals, to humans are known to the art.

For example, the concentration of the compounds in a liquid composition, such as a lotion, can be from about 0.1-25% by weight, or from about 0.5-10% by weight. The concentration in a semi-solid or solid composition such as a gel or a powder can be about 0.1-5% by weight, or about 0.5-2.5% by weight.

Effective dosages and routes of administration of agents of the invention are conventional. The exact amount (effective dose) of the agent will vary from subject to subject, depending on, for example, the species, age, weight and general or clinical condition of the subject, the severity or mechanism of any disorder being treated, the particular agent or vehicle used, the method and scheduling of administration, and the like. A therapeutically effective dose can be determined empirically, by conventional procedures known to those of skill in the art. See, e.g, The Pharmacological Basis of Therapeutics, Goodman and Gilman, eds., Macmillan Publishing Co., New York. For example, an, effective dose can be estimated initially either in cell culture assays or in suitable animal models. The animal model may also be used to determine the appropriate concentration ranges and routes of administration. Such information can then be used to determine useful doses and routes for administration in humans. A therapeutic dose can also be selected by analogy to dosages for comparable therapeutic agents.

The particular mode of administration and the dosage regimen will be selected by the attending clinician, taking into account the particulars of the case (e.g., the subject, the disease, the disease state involved, and whether the treatment is prophylactic). Treatment may involve daily or multi-daily doses of compound(s) over a period of a few days to months, or even years.

In general, however, a suitable dose will be in the range of from about 0.001 to about 100 mg/kg, e.g., from about 0.01 to about 100 mg/kg of body weight per day, such as above about 0.1 mg per kilogram, or in a range of from about 1 to about 10 mg per kilogram body weight of the recipient per day. For example, a suitable dose may be about 1 mg/kg, 10 mg/kg, or 50 mg/kg of body weight per day.

The compounds are conveniently administered in unit dosage form; for example, containing 0.05 to 10000 mg, 0.5 to 10000 mg, 5 to 1000 mg, or about 100 mg of active ingredient per unit dosage form. In some embodiments, the dosage unit contains about 1 mg, about 10 mg, about 25 mg, about 50 mg, about 75 mg, about 100 mg, about 150 mg, about 200 mg, about 250 mg, about 300 mg, about 350 mg, about 400 mg, about 450 mg, about 500 mg, about 750 mg, or about 1000 mg of active ingredient.

One aspect of the invention is a method for detecting the presence of amyloid (e.g., Abeta or tau) oligomers or fibers in a sample, comprising contacting a sample suspected of containing such oligomers or fibers with an effective amount of a detectably labeled compound of the invention and measuring the amount of (bound) label in the sample. Phrases such as “detecting an oligomer or fiber in a sample” are not meant to exclude samples or determinations (detection attempts) where no oligomer or fiber is contained or detected. In a general sense, this invention involves assays to determine whether the target is present in a sample, irrespective of whether or not it is detected.

As used herein, the singular forms “a,” “an,” and “the” include plural referents unless the context clearly dictates otherwise. Thus, “a” compound of the present invention, as used above, can be two or more compounds.

The contacting step can comprise, e.g., (1) taking a sample of body fluid or tissue (e.g., a suitable blood sample likely to contain amyloid molecules; (2) contacting the sample with a detectably labeled compound of the invention, under conditions effective for the compound to bind to the oligomers or fibers, e.g., reacting or incubating the sample and the compound; and (3) assaying the contacted sample for the presence of labeled compound which has bound to the amyloid oligomer or fiber.

Suitable labels which enable detection (e.g., provide a detectable signal, or can be detected), and methods for labeling compounds of the invention with the labels, are conventional and well-known to those of skill in the art. Suitable detectable labels include, e.g., radioactive active agents, fluorescent labels, and the like. Assays for detecting such labels are conventional

Conditions for binding a compound of the invention to an amyloid oligomer or fiber, and treating the sample as necessary to detect the targets to which the compound has bound, are conventional and well-known to those of skill in the art.

Suitable samples include tissues and bodily fluids, such as blood, cerebral spinal fluid (CSF), saliva, gastric secretions, mucus, or the like, which will be evident to a skilled worker;

In one aspect of the invention, amyloid oligomers or fibers are detected in a subject, e.g. in the brain of a subject. In one embodiment of the invention, a compound of the invention is labeled with a radionuclide which can be detected in a non-invasive manner. For example, if the compound is used in diagnosis according to single photon emission computed tomography (SPECT), examples of a radionuclide that can be used may include gamma-ray-emitting radionuclides such as ⁹⁹mTc, ¹¹¹In, ⁶⁷Ga, ²⁰1Tl, ¹²³I, or ¹³³Xe. When the compound is used in diagnosis according to Positron Emission Tomography (PET), examples of a radionuclide that can be used may include positron-emitting radionuclides such as ¹¹C, ¹³N, ¹⁵O, ¹⁸F, ⁶²Cu, 68Ga, or ⁷⁶Br. When the compound is administered to animals other than human, radionuclides having a longer half-life, such as ¹²⁵I, may also be used. Methods for formulating the labeled compounds, administering them to a subject, and imaging them with a suitable apparatus are conventional. Other labeled agents are currently being used to detect amyloid fibers in brain, and methods similar to those can be used with compounds of the present invention.

Another aspect of the invention is a kit for performing a method of the invention (e.g. for detecting amyloid in a sample or in a subject, or for inhibiting or reducing amyloid toxicity, in vitro or in a subject). The kit may comprise a suitable amount of a compound or pharmaceutical composition of the invention. Kits of the invention may comprise instructions for performing a method, such as a diagnostic method. Other optional elements of a kit of the invention include suitable buffers, media components, or the like; a computer or computer-readable medium for storing and/or evaluating the assay results; containers; or packaging materials. Reagents for performing suitable controls may also be included. The reagents of the kit may be in containers in which the reagents are stable, e.g., in lyophilized form or stabilized liquids. The reagents may also be in single use form, e.g., in single reaction form for diagnostic use.

In the foregoing and in the following examples, all temperatures are set forth in uncorrected degrees Celsius; and, unless otherwise indicated, all parts and percentages are by weight.

### EXAMPLES

### Example I

### Pharmacophores for Amyloid Fibers Involved in Alzheimer's Disease

### A. Materials and Methods

### Peptide and Compounds

Peptide segments (custom synthesis) were purchased from CS Bio. Orange-G and curcumin were purchased from Sigma-Aldrich. DDNP was synthesized as described in [38,58].

### Crystallizing Conditions

All crystals were grown at 18° C. via hanging-drop vapor diffusion. All crystals appeared within 1 wk, except the negative control crystals of VQIVYK (SEQ ID NO: 2)+DDNP that took 8 mo to grow.

VQIVYK (SEQ ID NO: 2)+orange-G. The drop was a mixture of 10 mM VQIVYK (SEQ ID NO: 2) and 1 mM orange-G in water, and reservoir solution (0.1 M zinc acetate dehydrate, 18% polyethylene glycol 335.0). The structure was solved to 1.8 Å resolution and contained one segment, one orange-G, two water molecules, two zinc atoms, and one acetate molecule in the asymmetric unit.
VQIVYK (SEQ ID NO: 2)+DDNP. The drop was a mixture of 6 mM VQIVYK (SEQ ID NO: 2) and 1 mM DDNP in 60% ethanol, and reservoir solution (0.52 M potassium sodium tartrate, 0.065 M HEPES-Na pH 7.5, 35% glycerol). The structure was solved to 1.2 Å resolution and contained one segment and three water molecules in the asymmetric unit.
VQIVYK (SEQ ID NO: 2)+DDNP from second crystallization conditions. The drop was a mixture of 6 mM VQIVYK (SEQ ID NO: 2) and 1 mM DDNP in 60% ethanol, and reservoir solution (1.2 M DL-malic acid pH 7.0, 0.1 M BIS-TRIS propane pH 7.0). The structure was solved to 1.65 Å resolution and contained one segment, and three water molecules in the asymmetric unit.
Negative control crystals to VQIVYK (SEQ ID NO: 2)+DDNP. The drop was a mixture of 6 mM VQIVYK (SEQ ID NO: 2) in 60% ethanol and reservoir solution (0.52 M potassium sodium tartrate, 0.065 M HEPES-Na pH 7.5, 35% glycerol). The structure was solved to 1.2 Å resolution and contained one segment, and one water molecule in the asymmetric unit.
VQIVYK (SEQ ID NO: 2)+curcumin. The drop was a mixture of 10 mM VQIVYK (SEQ ID NO: 2) and 1 mM curcumin in 80% dimethyl sulfoxide (DMSO), and reservoir solution (0.1 M Tris.HCl pH 8.5, 70% (v/v) MPD (2-methyl-2,4-pentanediol)). The structure was solved to 1.3 Å resolution and contained one segment, and two water molecules in the asymmetric unit.
Negative control crystals to VQIVYK (SEQ ID NO: 2)+curcumin. The drop was a mixture of 10 mM VQIVYK (SEQ ID NO: 2) in 80% DMSO and reservoir solution (0.1 M Tris.HCl pH 8.5, 70% (v/v) MPD (2-methyl-2,4-pentanediol)). The structure was solved to 1.3 Å resolution and contained one segment, and one water molecule in the asymmetric unit.
KLVFFA (SEQ ID NO: 1)+orange-G. The drop was a mixture of 10 mM KLVFFA (SEQ ID NO: 1) and 1 mM orange-G in water, and reservoir solution (10% w/v polyethylene glycol 1,500, 30% v/v glycerol). Another drop was a mixture of 5 mM KLVFFA (SEQ ID NO: 1) and 1 mM orange-G in water, and reservoir solution (30% w/v polyethylene glycol 1,500, 20% v/v glycerol). The structure was solved to 1.8 Å resolution and contained four segments, two orange-G molecules, and 11 water molecules in the asymmetric unit.
Negative control crystals to KLVFFA (SEQ ID NO: 1)+orange-G. The drop was a mixture of 10 mM KLVFFA (SEQ ID NO: 1) in water, and reservoir solution (10% w/v polyethylene glycol 1,500, 30% v/v glycerol). Another drop was a mixture of 5 mM KLVFFA (SEQ ID NO: 1) in water, and reservoir solution (30% w/v polyethylene glycol 1,500, 20% v/v glycerol). The structure was solved to 2.1 Å resolution and contained one segment and three water molecules in the asymmetric unit.

### Structure Determination and Refinement

X-ray diffraction data were collected at beamline 24-ID-E of the Advanced Photon Source (APS), Argonne National Laboratory; wavelength of data collection was 0.9792 Å. Data were collected at 100 K. Molecular replacement solutions for all segments were obtained using the program Phaser [59]. The search models consisted of available structures of the same segment or geometrically idealized n-strands. Crystallographic refinements were performed with the program Refmac5 [60]. Model building was performed with Coot [61] and illustrated with PyMOL [62]. There were no residues that fell in the disallowed region of the Ramachandran plot. Simulated annealing composite omit map was generated using CNS [63,64]; 10% was omitted.

### Computational Docking

Three-dimensional (3-D) structures of the small molecules were generated using Corina (Molecular Networks; http://www.molecular-networks.com/online_demos/corina_demo) and Chemical Identifier Resolver (http://cactus.nci.nih.gov/translate/). Additional 3-D conformations were generated using OpenEye Omega [65]. The small molecule was placed in approximate location according to the electron density map. The small molecule was docked to the peptide fibrillar structure using RosettaLigand [66,67]. The protein side chains were fixed. The generated docked structures (1,000 for KLVFFA (SEQ ID NO: 1)-orange-G and 500 for the rest of the structures) were further refined using Refmac5 [60] and the 10 best structures (based on lowest free-R [68]) were analyzed and showed to be very similar to each other. The best structures were further optimized and refined and the one with the lowest free-R was chosen as the final structure.

### Solvent Accessible Surface Area, Free Energy, and Dissociation Constant Calculations

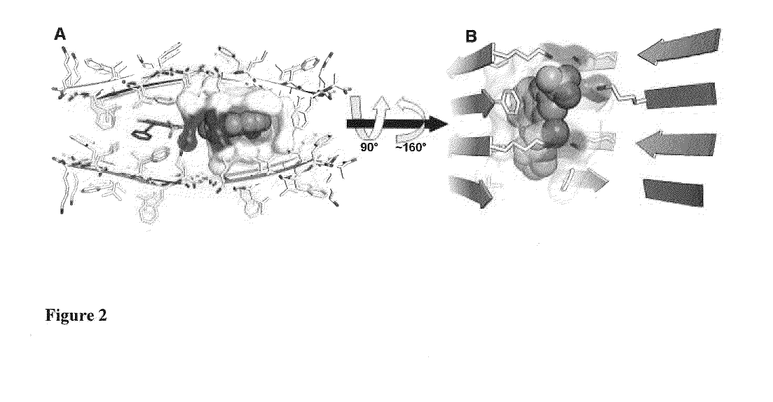

The area buried of the small molecules within the fiber structure was calculated using Areaimol [69,70] with a probe radius of 1.4 Å. The difference between the accessible surface areas of the fiber structure alone and with the small molecule constitutes the reported area buried. The Areaimol [69,70] calculations were also used to report the segment atoms that are in contact with the small molecules (shown in FIGS. 2, 5, and 7), and the percentage of apolar and polar contacts.

Binding energy and corresponding dissociation constant of one orange-G molecule to the KLVFFA (SEQ ID NO: 1) fiber were estimated from the apolar surface area (contributed by carbon atoms) that is covered by the interaction and was calculated using Areaimol [69,70]. The difference between the apolar accessible surface areas of the fiber structure atone and with the small molecule was added to the difference between the apolar accessible surface areas of the small molecule alone and with the fiber. These calculations resulted in 500 Å² of apolar surface area covered. The binding energy was calculated from the formula [71] ΔG⁰=18 cal×Å⁻²×mol⁻¹=18×500 cal/mol=9 kcal/mol. The dissociation constant was calculated from ΔG⁰−RT ln K. Thus, K=exp(−ΔG/RT)=3×10⁻⁷ M=0.3 μM.

### Mass Spectrometry Analysis of the Co-Crystals

Liquid chromatography tandem mass spectrometry (LC-MSMS) was used to measure the molar ratios of the peptide segments and the small molecules within the crystals. Authentic samples of the peptides and each of the small molecules were used to prepare standard response curves. Crystals from each of the four mixtures of peptides and small compounds were individually picked (using a sharpened glass capillary) and re-dissolved in 5%-10% acetonitrile. The samples were divided into two aliquots, one for the peptide analyses and the other for the small molecule analyses, and the amount of each component in the samples was interpolated using the standard curves.

Peptide standards (dry powder of VQIVYK (SEQ ID NO: 2) and KLVFFA (SEQ ID NO: 1)) were dissolved in water and prepared in concentrations ranging from 0.05 μM to 0.01 mM in 0.1% TFA. Aliquots of the standards and the re-dissolved crystals were separately injected (50 μL) onto a polymeric reverse phase column (PLRP/S, 2×150 mm, 5 μm, 300 Å; Varian) equilibrated in Buffer A (0.1% formic acid in water) and eluted (0.25 mL/min) with an increasing concentration of Buffer B (0.1% formic acid in acetonitrile). The effluent from the column was directed to an Ionspray source attached to a triple quadrupole mass spectrometer (Perkin Elmer/Sciex API III⁺) operating under previously optimized positive ion mode conditions. Data were collected in the positive ion multiple reaction monitoring (MRM) mode in which the intensity of specific parent→fragment ion transitions were recorded (VQIVYK (SEQ ID NO: 2), m/z 749.5→341.3, 749.5→409.4, 749.5→440.3, 749.5→522.5; KLVFFA (SEQ ID NO: 1), 724.4→84, 724.4→488.3, 362.7→84, 362.7→120.1).

Similar procedures were used for the analyses of the small molecules. Orange-G was dissolved in water and diluted with 10% ammonium acetate to concentrations ranging from 2 nM to 20 μM. Solutions of the standard and the re-dissolved crystals were separately injected (50 μL) onto a silica based reverse phase column (Supelco Ascentis Express C18, 150×2.1 mm, 2.7 μm) equilibrated in Buffer A (10 mM ammonium acetate) and eluted (0.2 mL/min) with an increasing concentration of Buffer B (acetonitrile/Isopropanol 1:1 containing 10 mM ammonium acetate). The negative ion MRM transitions were m/z 407.1→302.1 and 407.1→222.1.

DDNP was dissolved in 95% ethanol and diluted with 10% ammonium acetate to concentrations ranging from 2 nM to 20 μM. Solutions of the standards and the re-dissolved crystals (further diluted with acetonitrile:methanol:water:acetic-acid (41:23:36:1, v/v/v/v) to ensure dissolution) were separately injected (50 μL) onto a silica based reverse phase column (Supelco Ascentis Express C18, 150×2.1 mm, 2.7 μm) equilibrated in Buffer A (10 mM ammonium acetate) and eluted (0.2 mL/min) with an increasing concentration of Buffer B (acetonitrile/Isopropanol 1:1 containing 10 mM ammonium acetate). The positive ion MRM transition was: DDNP—m/z 262.1→247.1.

Curcumin was dissolved and diluted in acetonitrile:methanol:water:acetic-acid (41:23:36:1, v/v/v/v) to concentrations ranging from 2 nM to 2 μM. Aliquots of the standards and the re-dissolved crystals (further diluted with acetonitrile:methanol:water:acetic-acid (41:23:36:1, v/v/v/v) to ensure dissolution) were injected (100 μL) onto a silica based reverse phase column (Waters Symmetry Shield RP18 5 μM, 3.9×150 mm) equilibrated in Buffer A (10 mM ammonium acetate) and eluted (0.5 mL/min) with an increasing concentration of Buffer B (acetonitrile/Isopropanol 1:1 containing 10 mM ammonium acetate). The negative ion MRM transitions were m/z 367.1→173.1, 367.1→149.

B. Screening for Co-Crystals of Amyloid-Like Segments with Small Molecules

In our attempts to obtain complexes of small molecules with amyloid-like segments from disease-related proteins, we screened for co-crystals grown from dozens of mixtures (Table 1). The majority of the resulting crystals yielded X-ray diffraction too poor for structure determination. Others led to structure determinations of the small molecule or amyloid-like segment alone. Out of hundreds of co-crystallization trials (Table 1), four mixtures, described below, yielded co-crystals with suitable X-ray diffraction from segments of Aβ and tau with amyloid binders.

C. Crystal Structure of the KLVFFA (SEQ ID NO: 1) Segment from Aβ Complexed with Orange-G

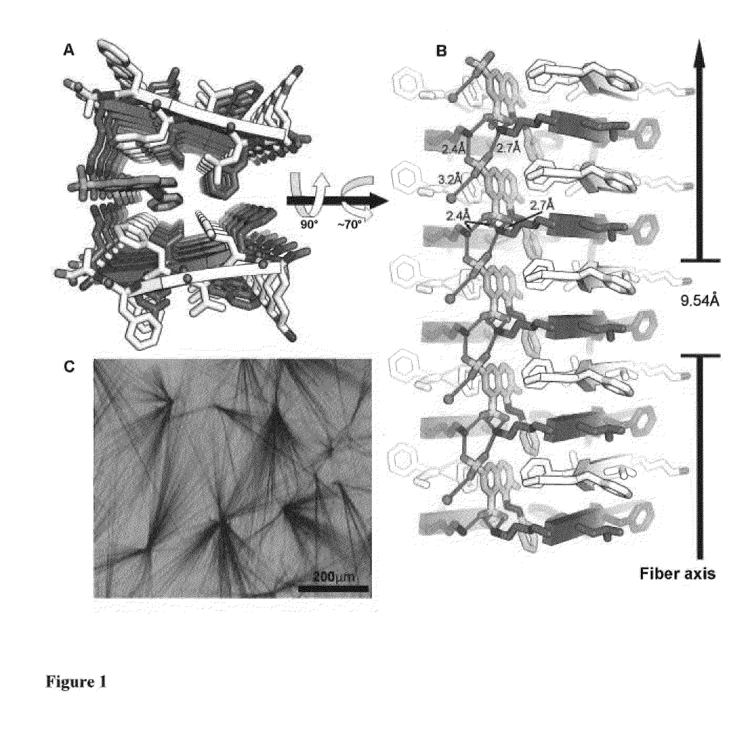

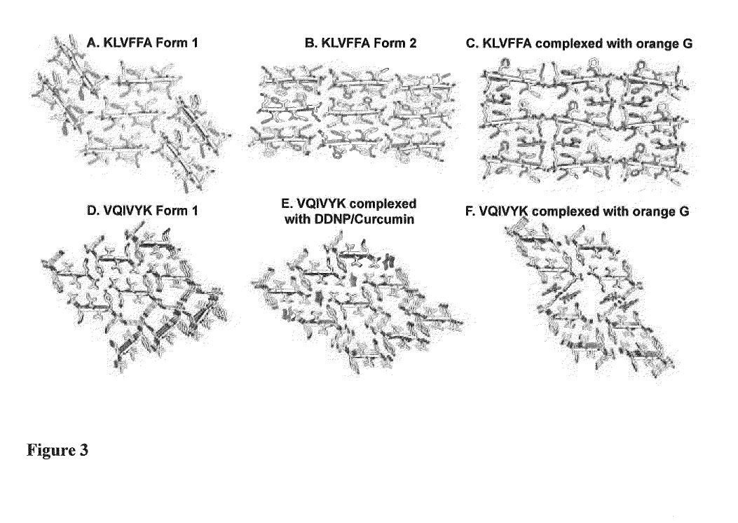

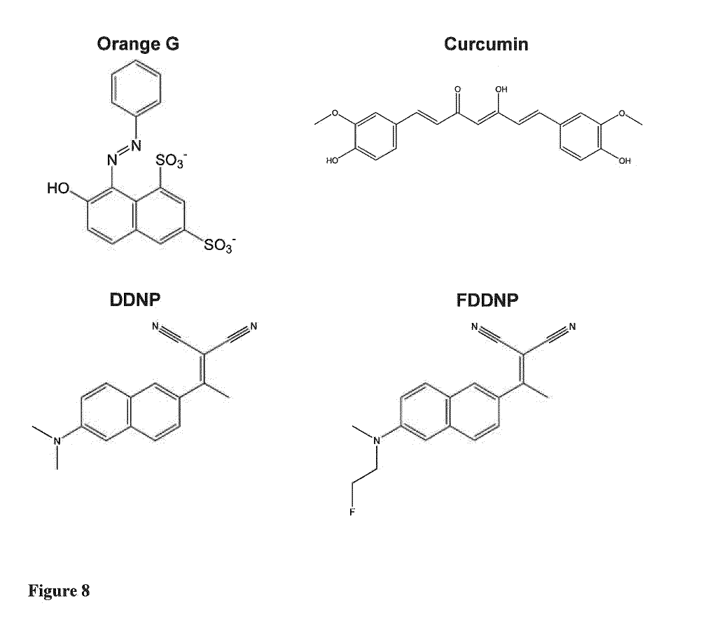

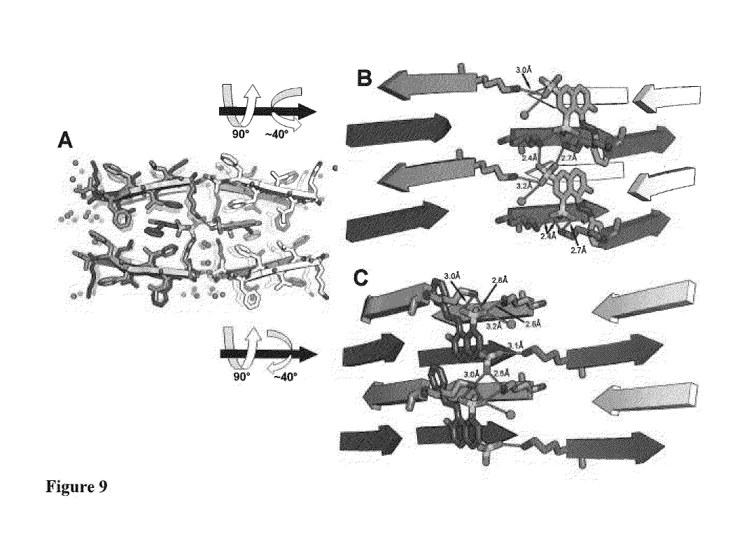

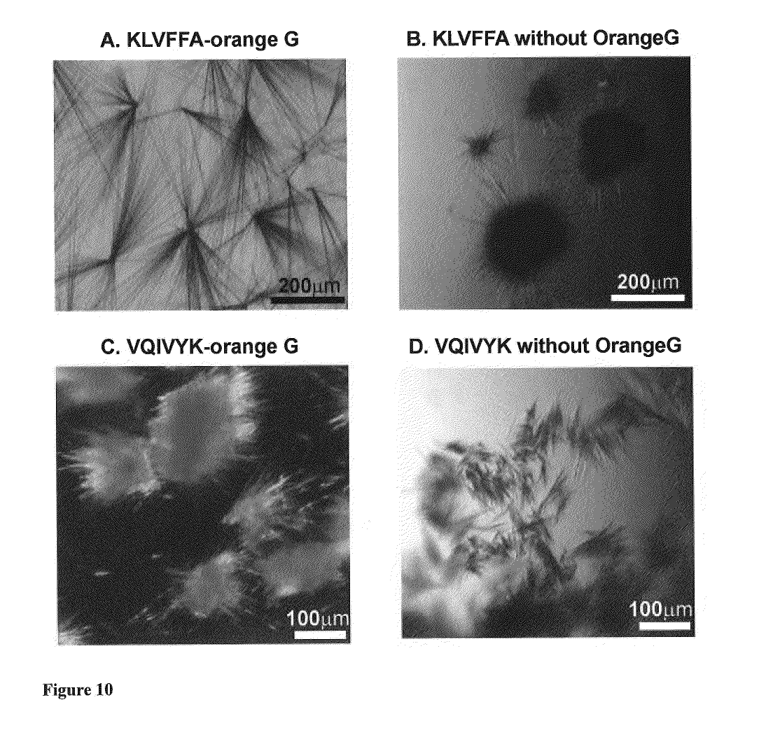

The KLVFFA (SEQ ID NO: 1) segment (residues 16-21) from Aβ contains apolar residues that participate in a hydrophobic spine in Aβ fibers and itself acts as an inhibitor of Aβ fibrillation [28,29]. We previously determined the atomic structure of the KLVFFA (SEQ ID NO: 1) segment in three crystal forms; all show the common steric zipper motif associated with amyloid fibers (Colletier et al. unpublished results). Orange-G (FIG. 8), a synthetic azo dye used in histological staining, affects the formation of Aβ fibers [7]. The co-crystallization of KLVFFA (SEQ ID NO: 1) with orange-G resulted in deeply colored crystals (FIG. 1C). Mass spectrometric analyses of the crystals showed high abundance of orange-G (˜1:1 molar stoichiometry with KLVFFA (SEQ ID NO: 1)). Determination of the structure revealed a novel, fourth form of the KLVFFA (SEQ ID NO: 1) steric zipper, with orange-G wedged between the paired β-sheets of the zipper, leading to partial opening of the zipper (FIGS. 1 and 9). Stabilization of the binding arises from packing of the aromatic rings of orange-G against the apolar, partially aromatic spine of KLVFFA (SEQ ID NO: 1) (FIG. 2). At the interface between orange-G and KLVFFA (SEQ ID NO: 1), a total of 500 Å² of apolar solvent-accessible surface area is covered, corresponding very roughly to a binding energy of 9 kcal/mol, or a dissociation constant of ˜0.3 μM (Methods). Further stabilization arises from the salt links between the negatively charged sulfonic acid groups of orange-G and positively charged lysine side chains from both β-sheets (FIGS. 1 and 9). Crystallization of KLVFFA (SEQ ID NO: 1) under identical conditions but without orange-G resulted in the formation of colorless crystals with a structure similar to Form-1 Colletier et al. (supra) results and FIGS. 3A and 10). Thus the binding of orange-G wedges apart the previously tightly mating pair of sheets of the steric zipper.

All four crystal forms of KLVFFA (SEQ ID NO: 1), including the complex with orange G, show an anti-parallel β-strand stacking in the steric zipper (Colletier et al. (supra) and FIG. 1). Nuclear magnetic resonance (NMR) characterization of Aβ fibers suggested a parallel orientation of the full-length Aβ [30]. Yet an anti-parallel orientation was proposed for various Aβ segments, both in the region of the KLVFFA (SEQ ID NO: 1) segment (residue numbers are indicated in subscript): Aβ_{16-22 [}31], Aβ_{17-21 [}32], and Aβ_{11-25 [}32], as well as for a segment at the C-terminus: Aβ_{34-42 [}33]. Moreover, the “Iowa” Aβ mutant that is related to a familial, early onset, Alzheimer's disease [34] also displays an anti-parallel β-strand orientation. Of potential importance, Aβ oligomers were also suggested to form anti-parallel β-sheet structures [35,36].

D. Crystal Structures of the VQIVYK (SEQ ID NO: 2) Segment from the Tau Protein with Orange-G

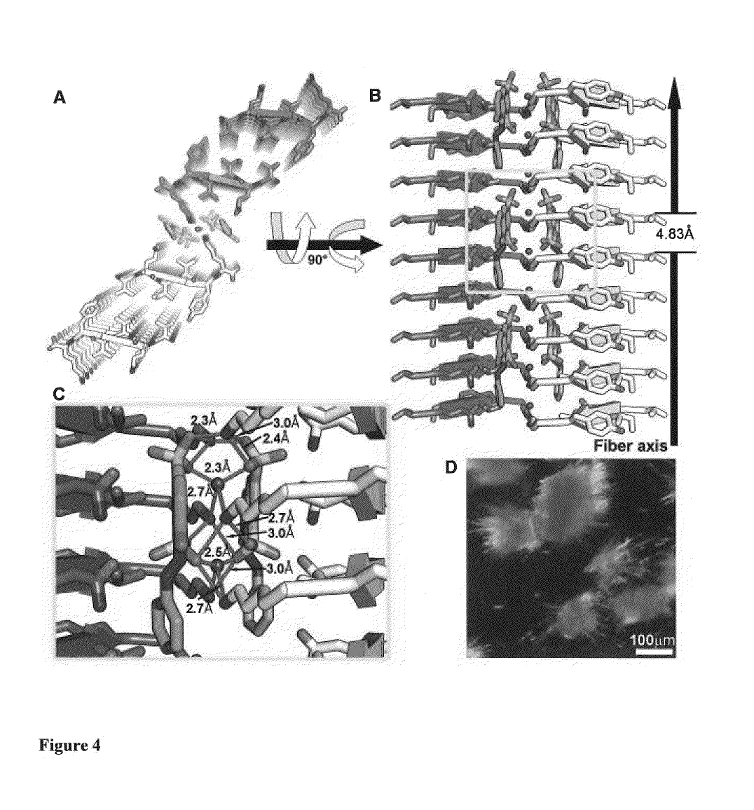

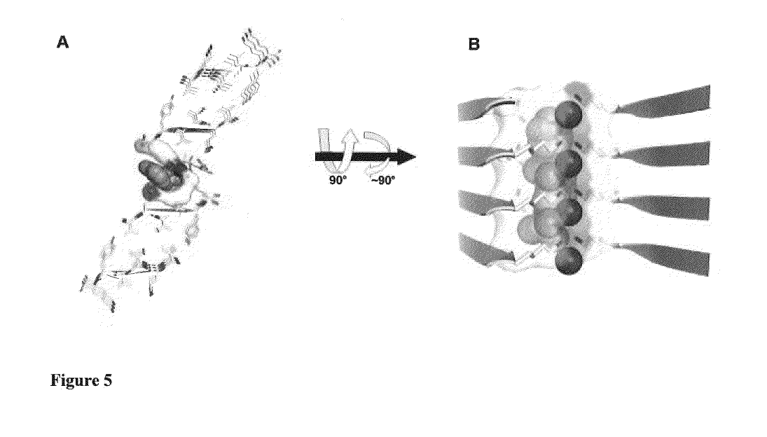

The VQIVYK (SEQ ID NO: 2) segment of tau was suggested as the minimal interaction motif for fiber formation [37]. We previously determined the crystal structure of VQIVYK (SEQ ID NO: 2) in two crystal forms; both show the common steric zipper motif of amyloid fiber-like structures [18,25]. Co-crystallization of VQIVYK (SEQ ID NO: 2) with orange-G resulted in deep orange crystals (FIG. 4D). Mass spectrometric analyses of the crystals showed relatively high abundance of orange-G (˜1:10 molar stoichiometry with VQIVYK (SEQ ID NO: 2)). Determination of the structure revealed a new crystal form of VQIVYK (SEQ ID NO: 2) (FIG. 4). Similar to Form-1 [18], the steric zipper shows a tight and dry interface; yet there is a large void between pairs of steric zipper, in contrast to the tightly packed structure of Form-1 (FIG. 3D). Orange-G is situated within this void, binding between lysine side chains facing each other from two parallel pairs of zippers, forming an electrostatic network that also involves zinc cations (FIGS. 4-5). As in its complex with KLVFFA (SEQ ID NO: 1), orange-G lies with its long axis parallel to the fiber axis.

Crystallization of VQIVYK (SEQ ID NO: 2) alone, under identical conditions to the co-crystallization of the VQIVYK (SEQ ID NO: 2)-orange-G mixture, resulted in the formation of colorless fibrous crystals (FIG. 10) giving poor X-ray diffraction. Under these conditions, the presence of orange-G appears crucial for the formation of well-ordered crystals.

E. Crystal Structures of the VQIVYK (SEQ ID NO: 2) Segment from the Tau Protein with Curcumin and DDNP

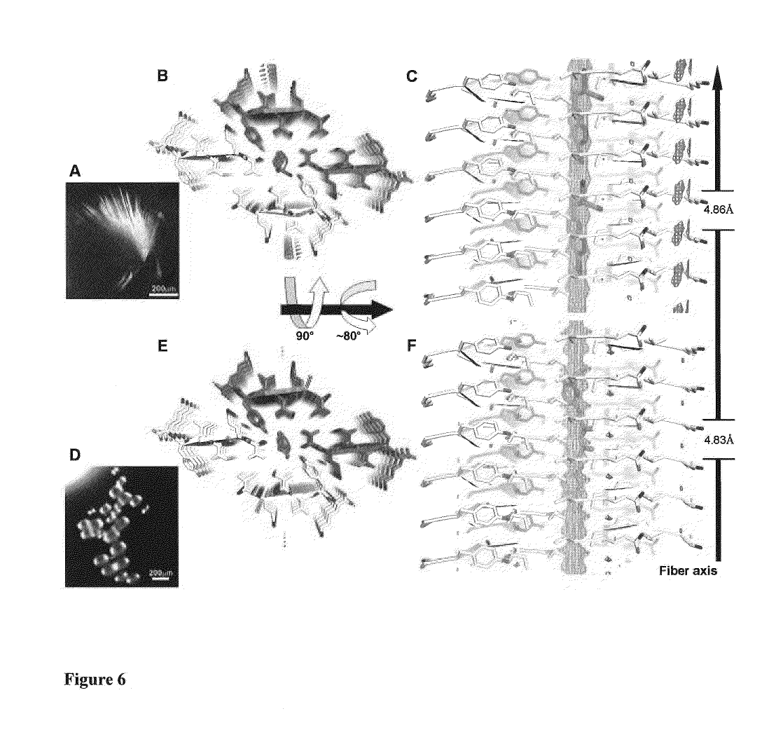

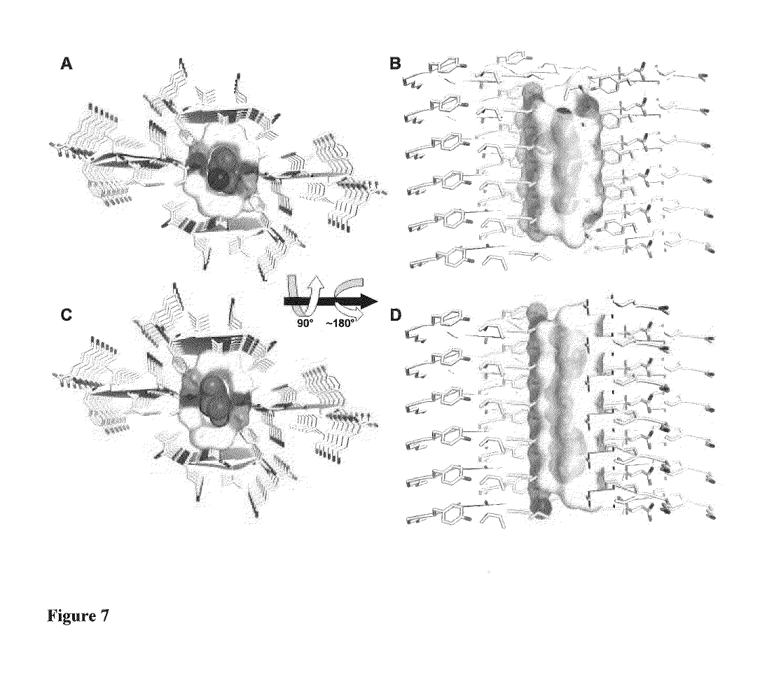

Curcumin (FIG. 8) from the plant turmeric protects neuronal cells against amyloid toxicity [9]. DDNP (FIG. 8) [38] and its analogs, synthetic diagnostics, bind Alzheimer's-associated neurofibrillary tangles and β-amyloid senile plaques and are used for the detection of plaques in the brains of Alzheimer's disease patients [39,40]. Co-crystallization of VQIVYK (SEQ ID NO: 2) with either curcumin or DDNP resulted in yellowish crystals (FIG. 6). Similar to Form-2 of VQIVYK (SEQ ID NO: 2) [25], the structures of VQIVYK (SEQ ID NO: 2) complexed with either curcumin or DDNP revealed that in both complexes, each member of a pair of β-sheets is shifted relative to the other, partially eliminating the dry interface in the steric zipper structure (FIGS. 3E and 6). In both complexes, the electron density attributed to the small molecule (either curcumin or DDNP) lies along the void left by the shifting of the steric zipper. This electron density is too undifferentiated to model the small molecule in atomic detail. However, it shows that the long axes of both curcumin and DDNP lie parallel to the fiber axis (FIGS. 6-7), as in the KLVFFA (SEQ ID NO: 1) and VQIVYK (SEQ ID NO: 2) structures with orange-G.

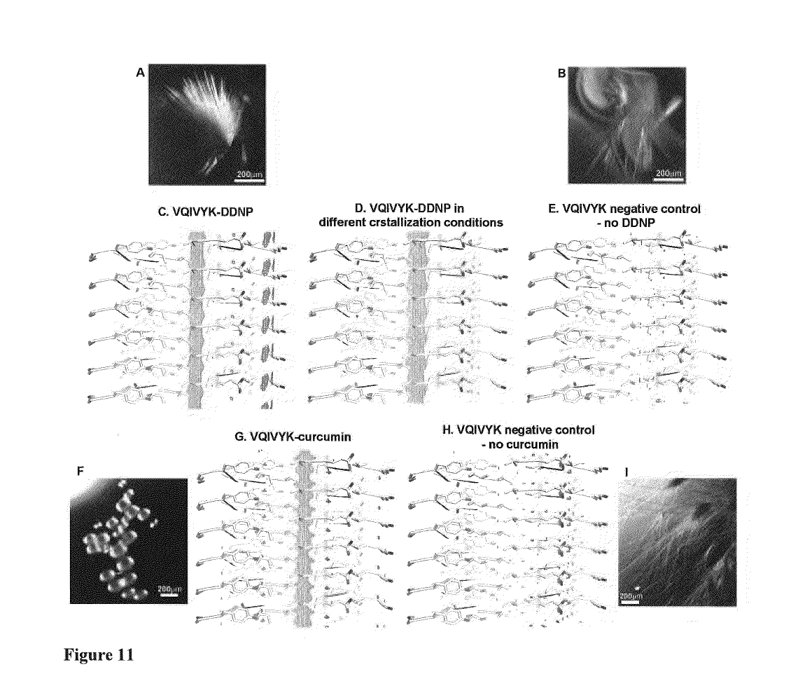

Despite the lack of differentiated electron density for curcumin and DDNP in VQIVYK (SEQ ID NO: 2), there is strong evidence for the presence of the small molecules in the crystals. The crystals show a distinctive color, whereas the control crystals (grown under identical condition without the small molecule) are colorless (FIG. 11). The control crystals also lack the additional positive density attributed to the small molecule (FIG. 11). Co-crystals of VQIVYK (SEQ ID NO: 2) and DDNP grown under alternative crystallization conditions showed a similar positive electron density (FIG. 11D), supporting its attribution to DDNP. Furthermore, the crystals grown in the presence of DDNP appeared within days, whereas the control crystals grew only after 8 months, suggesting that the presence of DDNP is a catalyst for crystallization. The strongest evidence supporting the presence of the small molecules in the structure is provided by mass spectrometric analyses of the crystals. The analyses also provided the reasoning for the undifferentiated electron density, showing a very low molar abundance of both curcumin and DDNP in the crystal (˜100 and ˜400 VQIVYK (SEQ ID NO: 2) segments for each curcumin or DDNP molecule, respectively), which is in close approximation to the experimentally established molar ratio between FDDNP (the fluoridated version of DDNP) (FIG. 8) and Aβ fibril of 1:1500 to 1:3000 [41]. We conjecture that the lack of site anchoring of the hydrophobic, uncharged small molecules to specific residues in the fibril leads to undifferentiated electron density. Furthermore, the nature of the binding site (a narrow tube running along the β-sheets) (FIGS. 6-7) implies that the apolar small molecules are free to drift along the fiber axis (see Example II).

The common feature of the structures of four amyloid/small-molecule complexes is that the small molecules bind to fibers in a similar orientation, along the β-sheets, with their long axes parallel to the fiber axis. This orientation was previously proposed for the binding of thioflavin T to bovine insulin and bovine β-lactoglobulin amyloid fibrils using polarized laser confocal microscopy [42]. A similar mode of binding was seen in co-crystals of oligomer-like β-2-microglubulin with thioflavin T, showing that thioflavin T is bound between β-sheets, orthogonal to the β-strands [43]. The orientation of congo-red was also suggested to be parallel to the amyloid long axis based on electron diffraction, linear dichroism [44], and a recent NMR-based model of congo-red bound to the fungal prion domain HET-s (218-289) [45].

### F. Discussion

Our crystal structures of small molecules bound within amyloid-like steric zippers define molecular frameworks, or pharmacophores, for the design of diagnostics and drugs for Alzheimer's and other aggregation diseases. The amyloid components in our structures are steric zippers formed by stacks of six-residue segments from Alzheimer-related proteins. Although these steric zippers cannot represent all aspects of the full-length amyloid parent proteins, they share many properties and are commonly used as models of the amyloid β-spine and of aggregation [22,24]. The small molecules in our structures bind along the 3-spine, and because the parent amyloids contain the same segments, we expect a similar mode of binding along the spine of the full-length parent amyloid fibers. Moreover, we expect the steric zipper spine of the parent fibers to be flanked with the rest of the protein residues in a native-like or unfolded conformation [12,20] and therefore to contain more solvent channels, or accessible sites for the binding of the small molecules, compared to the very compact packing of the steric zipper segments. Consistently, orange-G, curcumin, and DDNP all bind to, or affect fibrillation of, full-length fibers [7,9,39].

### Molecular Frameworks of Amyloid Binders

Overall, the complexes presented here define two molecular frameworks for the binding of small molecules to amyloid fibers. The first molecular framework pertains to site-specific binders, such as charged compounds that form networks of interactions with sequence motifs, and is relatively well defined. The second molecular framework, far less well defined at this point, pertains to broad-spectrum binders, such as uncharged aromatic compounds that bind to tube-like cavities between β-sheets. Without wishing to be bound by any particular mechanism, it is suggested that for binding amyloid deposits in the brain, uncharged molecules may be more effective because of superior blood-brain-barrier penetrability. The same frameworks, offering cavities along β-sheets, are also expected to exist in amyloid oligomers known to be rich in β-sheets and possibly fiber-like [46], similar to the observed binding of amyloid markers to β-sheets in non-fibrillar structures [43,47]. Consistent with this, both oligomers and fibers are inhibited by similar compounds, including curcumin [7,9].

The specific binding of orange-G allows definition of the chemical properties of a specific molecular framework. The prominent feature of amyloid structures is the separation of β-strands (forming a (β-sheet) by ˜4.8 Å. In structures with strands packed in an antiparallel orientation, as observed for the KLVFFA (SEQ ID NO: 1) fibers and for a rare mutation in Aβ that is associated with massive depositions of the mutant protein and early onset of the disease [34,48], the separation of repeating units (2 strands) is twice as great, ˜9.6 Å. Orange-G contains two negatively charged sulfonic acid groups facing the same direction, with the sulfur atoms spaced ˜5 Å apart and the oxygen atoms separated by 4.5-7.5 Å. This framework allows the formation of salt links between the sulfonic acid groups and lysine ammonium ions from every repeating strand in both KLVFFA (SEQ ID NO: 1) (anti-parallel orientation) and VQIVYK (SEQ ID NO: 2) (parallel orientation) fibers (FIGS. 1 and 4). This shows that a specific framework includes two charged moieties spaced either ˜4.8 Å or ˜9.6 Å apart. The specific sequence motif of the spine of the fiber and the separation of the β-strands dictates the signs of the necessary charges in the small molecule and their separation.

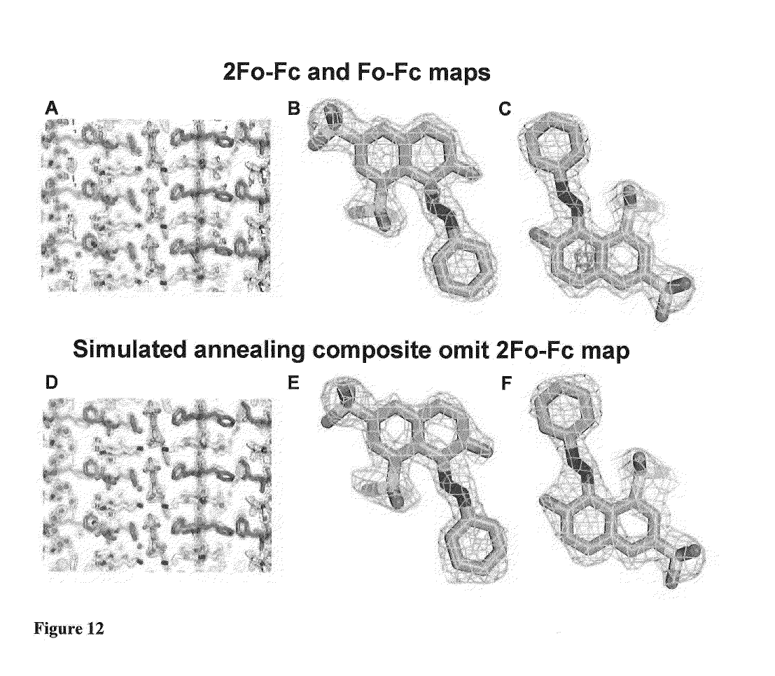

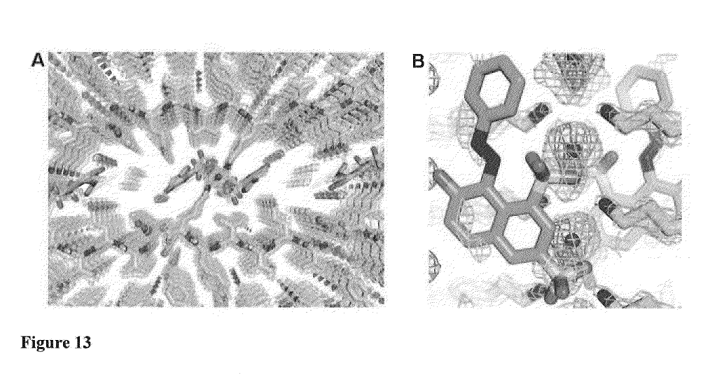

Within our framework, an apolar aromatic spine is another essential moiety [22]. The largely apolar KLVFFA (SEQ ID NO: 1) segment attracts the apolar surface of orange-G, stabilizing the binding (FIG. 2). In the complex with VQIVYK (SEQ ID NO: 2), the aromatic rings of orange-G are also packed against apolar side chains, but the binding is largely mediated via polar interactions with glutamine and lysine side-chains at the edges of two steric zippers (FIG. 5). The differences in the binding cavities between the KLVFFA (SEQ ID NO: 1) and VQIVYK (SEQ ID NO: 2) fibers may account for the higher molecular stoichiometry within the KLVFFA (SEQ ID NO: 1)-orange-G crystals observed by mass spectrometric analyses, and the correspondingly greater order of this complex (FIGS. 12-13).

Despite the lack of atomized electron density for the binding of curcumin and DDNP in VQIVYK (SEQ ID NO: 2) fibers, the location of the binding cavity is clear. It is narrow, restricting rotation of the small molecule (FIGS. 6-7). The atomic groups lining the binding cavity are about half apolar and half polar (FIG. 7). The tube-like shape favors the binding of uncharged molecules, such as DDNP and curcumin. The binding site is, however, insufficiently site-specific to allow for high occupancy and ordered interactions and is not yet well defined in atomic detail.

Our structures show that different small molecules bind along β-spine of amyloid-like fibers. In case fibers contain more than a single spine, the molecules might bind to multiple sites. This is more likely for the broad-spectrum hydrophobic compounds but can also apply for charged compounds. For example, we observed orange-G to bind to two different steric zippers, of KLVFFA (SEQ ID NO: 1) and VQIVYK (SEQ ID NO: 2), with the commonality of binding to lysine side chains protruding from the β-sheets.

Congo-red, a known amyloid marker, contains two sulfonic acid groups, similar to orange-G, but they are spaced ˜19 Å apart, which might account for its lack of specificity [44]. In a recent model, built using NMR constrains, congo-red was computationally docked to the fungal prion domain HET-s (218-289), suggesting that the sulfonic acid groups interact with lysine residues protruding from the sheets [45], similar to orange-G in our structures. However, in the model, the strands of HET-s are arranged in an anti-parallel orientation and the sulfonic acid groups of congo-red interact with every other lysine along the fiber [45], while orange-G interacts with every single lysine in both the KLVFFA (SEQ ID NO: 1) and VQIVYK (SEQ ID NO: 2) complexes (FIGS. 1 and 4). Both congo-red and thioflavin T, another known marker, bind to numerous different β-structures, even in a non-fibrillar form [43,47]. Despite their limited specificity and low affinity [49,50], these dyes play a major role in amyloid research because their binding is detectible via birefringence or fluorescence [51,52]. An important application of our structures is for the design of new markers for aggregation that will be more potent and can also be used in vivo.

### The Two Molecular Frameworks and Function

Defining these two molecular frameworks illuminates functional attributes of specific and broad-spectrum amyloid binders. This distinction is consistent with competitive kinetic experiments demonstrating that the binding of FDDNP (the fluoridated analog of DDNP) to Aβ fibrils is displaceable by the uncharged non-steroidal anti-inflammatory naproxen, but not by the common charged dyes congo-red and thioflavin T [53]. Moreover, in vitro FDDNP labels amyloid-like structures in a fashion similar to congo-red and thioflavin T, providing further evidence for the broad-spectrum type of binding [54]. Knowledge of both frameworks can lead to the design of more potent and specific compounds. Without wishing to be bound by any particular mechanism, it is suggested that these molecules can act as binders and be used as diagnostics, or serve as inhibitors of aggregation by either destabilizing steric zippers by wedge action (FIG. 1) or binding between steric zippers preventing higher-order β-sheet interactions (FIG. 4).

In the case of the complexed curcumin and DDNP structures, we expect that the tube-like cavity along the β-sheets provides an adequate site for the binding of many compounds of similar properties. However, the lack of specific interactions allows the small molecule to drift along the fiber axis, leading to lower occupancy and a degree of fluidity in the structure. Extrapolating from our structures, we expect that various aromatic compounds, such as polyphenols [6], would bind to a variety of amyloid-forming sequences because of a cylindrical, partially apolar cavity that forms between the pairs of β-sheets forming the fibers. These cavities are also expected to provide binding sites for various kinds of apolar drugs, such as benzodiazepines and anesthetics, explaining some of the altered pharmacokinetic properties and increased sensitivity detected in elderly [55].

One implication of our structures for the design of effective therapeutic treatments is the specificity they reveal of ligand binding to particular fiber polymorphs (FIG. 3). Various amyloid proteins show diverse fiber morphologies that are correlated with different patterns of pathology and toxicity [56,57]. In earlier work, we have suggested that fiber polymorphism has its molecular basis in different steric zippers (β-sheet packing) formed by the same sequence [25]. Our new findings show that different compounds bind to different fiber polymorphs formed by the same sequence. For example, orange-G displaces one VQIVYK (SEQ ID NO: 2) zipper relative to its mate; that is, wedges between protofilaments (FIGS. 3F and 4). In contrast, both DDNP and curcumin opportunistically bind to cylindrical cavities at the edges of VQIVYK (SEQ ID NO: 2) zippers, in a void formed within a different VQIVYK (SEQ ID NO: 2) β-sheet packing (FIGS. 3E and 6). This suggests that each compound binds to only a sub-population of fibers. Thus, just as cocktails of anti-HIV drugs are necessary to inhibit different viral strains, a combination of compounds may be necessary to bind to the several amyloid polymorphs present.

### G. Conclusions

Four crystal structures of small molecules bound to fiber-forming segments of the two main Alzheimer's disease proteins show common features. The small molecules bind with their long axes parallel to the fiber axis. The structures reveal a sequence-specific binder which forms salt links with side-chains of the steric zipper spines of the fibers and non-specific binders which lie in cylindrical cavities formed at the edges of several steric zippers. Small-molecule binding is specific to particular steric-zipper polymorphs, suggesting that for effective Alzheimer's diagnostics and therapeutics, it may be advantageous to have to be mixtures of various compounds to bind to all polymorphs present. The complexes presented here providet routes for structure-based design of combinations of compounds that can bind to a spectrum of polymorphic aggregates, to be used as markers of fibers and as inhibitors of aggregation.

### REFERENCES FOR EXAMPLE I

1. Glenner G G, Wong C W (1984) Alzheimer's disease: initial report of the purification and characterization of a novel cerebrovascular amyloid protein. Biochem Biophys Res Commun 120: 885-890.
2. Steiner B, Mandelkow E M, Biernat J, Gustke N, Meyer H E, et al. (1990) Phosphorylation of microtubule-associated protein tau: identification of the site for Ca2(+)-calmodulin dependent kinase and relationship with tau phosphorylation in Alzheimer tangles. EMBO J 9: 3539-3544.
3. Lesne S, Koh M T, Kotilinek L, Kayed R, Glabe C G, et al. (2006) A specific amyloid-beta protein assembly in the brain impairs memory. Nature 440: 352-357.
4. Collins S R, Douglass A, Vale R D, Weissman J S (2004) Mechanism of prion propagation: amyloid growth occurs by monomer addition. PLoS Biol 2: e321. doi:10.1371/journal.pbio.0020321
5. Maezawa I, Hong H S, Liu R, Wu C Y, Cheng R H, et al. (2008) Congo red and thioflavin-T analogs detect Abeta oligomers. J Neurochem 104: 457-468.
6. Porat V, Abramowitz A, Gazit E (2006) Inhibition of amyloid fibril formation by polyphenols: structural similarity and aromatic interactions as a common inhibition mechanism. Chem Biol Drug Des 67: 27-37.
7. Necula M, Kayed R, Milton S, Glabe C G (2007) Small molecule inhibitors of aggregation indicate that amyloid beta oligomerization and fibrillization pathways are independent and distinct. J Biol Chem 282: 10311-10324.
8. Bulic B, Pickhardt M, Mandelkow E M, Mandelkow E (2010) Tau protein and tau aggregation inhibitors. Neuropharmacology 59: 276-289.
9. Yang F, Lim G P, Begum A N, Ubeda O J, Simmons M R, et al. (2005) Curcumin inhibits formation of amyloid beta oligomers and fibrils, binds plaques, and reduces amyloid in vivo. J Biol Chem 280: 5892-5901.
10. Hong H S, Maezawa I, Yao N, Xu B, Diaz-Avalos R, et al. (2007) Combining the rapid MTT formazan exocytosis assay and the MC65 protection assay led to the discovery of carbazole analogs as small molecule inhibitors of Abeta oligomer-induced cytotoxicity. Brain Res 1130: 223-234.
11. Bayro M J, Maly T, Birkett N R, Macphee C E, Dobson C M, et al. (2010) High-resolution MAS NMR analysis of PI3-SH3 amyloid fibrils: backbone conformation and implications for protofilament assembly and structure. Biochemistry 49: 7474-7484.
12. Nelson R, Eisenberg D (2006) Recent atomic models of amyloid fibril structure. Curr Opin Struct Biol 16: 260-265.
13. Lührs T, Ritter C, Adrian M, Riek-Loher D, Bohrmann B, et al. (2005) 3D structure of Alzheimer's amyloid-beta(1-42) fibrils. Proc Natl Acad Sci USA 102: 17342-17347.
14. Petkova A T, Ishii Y, Balbach J J, Antzutkin O N, Leapman R D, et al. (2002) A structural model for Alzheimer's beta-amyloid fibrils based on experimental constraints from solid state NMR. Proc Natl Acad Sci USA 99: 16742-16747.
15. Paravastu A K, Leapman R D, Yau W M, Tycko R (2008) Molecular structural basis for polymorphism in Alzheimer's β-amyloid fibrils. Proc Natl Acad Sci USA 105: 18349-18354.
16. Kirschner D A, Abraham C, Selkoe D J (1986) X-ray diffraction from intraneuronal paired helical filaments and extraneuronal amyloid fibers in Alzheimer disease indicates cross-beta conformation. Proc Natl Acad Sci USA 83: 503-507.
17. Astbury W T, Beighton E, Parker K D (1959) The cross-beta configuration in supercontracted proteins. Biochim Biophys Acta 35: 17-25.
18. Sawaya M R, Sambashivan S, Nelson R, Ivanova M I, Sievers S A, et al. (2007) Atomic structures of amyloid cross-beta spines reveal varied steric zippers. Nature 447: 453-457.
19. Nelson R, Sawaya M R, Balbirnie M, Madsen AØ, Riekel C, et al. (2005) Structure of the cross-β spine of amyloid-like fibrils. Nature 435: 773-778.
20. Sambashivan S, Liu Y, Sawaya M R, Gingery M, Eisenberg D (2005) Amyloid-like fibrils of ribonuclease A with three-dimensional domain-swapped and native-like structure. Nature 437: 266-269.
21. Ivanova M I, Thompson M J, Eisenberg D (2006) A systematic screen of beta(2)-microglobulin and insulin for amyloid-like segments. Proc Natl Acad Sci USA 103: 4079-4082.
22. Gazit E (2005) Mechanisms of amyloid fibril self-assembly and inhibition. Model short peptides as a key research tool. FEBS J 272: 5971-5978.
23. Teng P K, Eisenberg D (2009) Short protein segments can drive a non-fibrillizing protein into the amyloid state. Protein Eng Des Sel 22: 531-536.
24. Gazit E (2002) Mechanistic studies of the process of amyloid fibrils formation by the use of peptide fragments and analogues: implications for the design of fibrillization inhibitors. Current Medicinal Chemistry 9: 1725-1735.
25. Wiltzius J J, Landau M, Nelson R, Sawaya M R, Apostol M I, et al. (2009) Molecular mechanisms for protein-encoded inheritance. Nat Struct Mol Biol 16: 973-978.
26. Wiltzius J J, Sievers S A, Sawaya M R, Cascio D, Popov D, et al. (2008) Atomic structure of the cross-β spine of islet amyloid polypeptide (amylin). Protein Sci 17: 1467-1474.
27. Apostol M I, Sawaya M R, Cascio D, Eisenberg D (2010) Crystallographic studies of prion protein (PrP) segments suggest how structural changes encoded by polymorphism at residue 129 modulate susceptibility to human prion disease. J Biol Chem 285: 29671-29675.
28. Tjernberg L O, Naslund J, Lindqvist F, Johansson J, Karlstrom A R, et al. (1996) Arrest of beta-amyloid fibril formation by a pentapeptide ligand. J Biol Chem 271: 8545-8548.
29. Chalifour R J, McLaughlin R W, Lavoie L, Morissette C, Tremblay N, et al. (2003) Stereoselective interactions of peptide inhibitors with the β-amyloid peptide. J Biol Chem 278: 34874-34881.
30. Antzutkin O N, Balbach J J, Leapman R D, Rizzo N R, Reed J, et al. (2000) Multiple quantum solid-state NMR indicates a parallel, not antiparallel, organization of beta-sheets in Alzheimer's beta-amyloid fibrils. Proc Natl Acad Sci USA 97: 13045-13050.
31. Balbach J J, Ishii Y, Antzutkin O N, Leapman R D, Rizzo N W, et al. (2000) Amyloid fibril formation by A beta 16-22, a seven-residue fragment of the Alzheimer's beta-amyloid peptide, and structural characterization by solid state NMR. Biochemistry 39: 13748-13759.
32. Petkova A T, Buntkowsky G, Dyda F, Leapman R D, Yau W M, et al. (2004) Solid state NMR reveals a pH-dependent antiparallel beta-sheet registry in fibrils formed by a beta-amyloid peptide. J Mol Biol 335: 247-260.
33. Lansbury P T, Jr., Costa P R, Griffiths J M, Simon E J, Auger M, et al. (1995) Structural model for the beta-amyloid fibril based on interstrand alignment of an antiparallel-sheet comprising a C-terminal peptide. Nat Struct Biol 2: 990-998.
34. Tycko R, Sciarretta K L, Orgel JPRO, Meredith S C (2009) Evidence for novel-sheet structures in Iowa-mutant-amyloid fibrils. Biochemistry 48: 6072-6084.
35. Cerf E, Sarroukh R, Tamamizu-Kato S, Breydo L, Derclaye S, et al. (2009) Antiparallel beta-sheet: a signature structure of the oligomeric amyloid beta-peptide. Biochem J 421: 415-423.
36. Yu L, Edalji R, Harlan J E, Holzman T F, Lopez A P, et al. (2009) Structural characterization of a soluble amyloid beta-peptide oligomer. Biochemistry 48: 1870-1877.
37. von Bergen M, Friedhoff P, Biernat J, Heberle J, Mandelkow E M, et al. (2000) Assembly of tau protein into Alzheimer paired helical filaments depends on a local sequence motif ((306)VQIVYK (SEQ ID NO: 2) (311)) forming beta structure. Proc Natl Acad Sci USA 97: 5129-5134.
38. Jacobson A, Petric A, Hogenkamp D, Sinur A, Barrio J R (1996) 1,1-Dicyano-2-[6-(dimethylamino)naphthalen-2-yl]propene (DDNP): A solvent polarity and viscosity sensitive fluorophore for fluorescence microscopy. J Am Chem Soc 118: 5572-5579.
39. Agdeppa E D, Kepe V, Liu J, Small G W, Huang S C, et al. (2003) 2-Dialkylamino-6-acylmalononitrile substituted naphthalenes (DDNP analogs): novel diagnostic and therapeutic tools in Alzheimer's disease. Mol Imaging Biol 5: 404-417.
40. Shoghi-Jadid K, Small G W, Agdeppa E D, Kepe V, Ercoli L M, et al. (2002) Localization of neurofibrillary tangles and beta-amyloid plaques in the brains of living patients with Alzheimer disease. Am J Geriatr Psychiatry 10: 24-35.
41. Agdeppa E D, Kepe V, Liu J, Flores-Torres S, Satyamurthy N, et al. (2001) Binding characteristics of radiofluorinated 6-dialkylamino-2-naphthylethylidene derivatives as positron emission tomography imaging probes for beta-amyloid plaques in Alzheimer's disease. J Neurosci 21: RC 189.
42. Krebs M R, Bromley E H, Donald A M (2005) The binding of thioflavin-T to amyloid fibrils: localisation and implications. J Struct Biol 149: 30-37.
43. Wolfe L S, Calabrese M F, Nath A, Blaho D V, Miranker A D, et al. (2010) Protein-induced photophysical changes to the amyloid indicator dye thioflavin T. Proc Natl Acad Sci USA 107: 16863-16868.
44. Childers W S, Mehta A K, Lu K, Lynn D G (2009) Templating molecular arrays in amyloid's cross-beta grooves. J Am Chem Soc 131: 10165-10172.
45. Schütz A K, Soragni A, Homemann S, Aguzzi A, Ernst M, et al. (2011) The amyloid-Congo red interface at atomic resolution. Angew Chem Int Ed in press.
46. Sandberg A, Luheshi L M, Sollvander S, Pereira de Barros T, Macao B, et al. (2010) Stabilization of neurotoxic Alzheimer amyloid-beta oligomers by protein engineering. Proc Natl Acad Sci USA 107: 15595-15600.
47. Turnell W G, Finch J T (1992) Binding of the dye congo red to the amyloid protein pig insulin reveals a novel homology amongst amyloid-forming peptide sequences. J Mol Biol 227: 1205-1223.
48. Van Nostrand W E, Melchor J P, Cho H S, Greenberg S M, Rebeck G W (2001) Pathogenic effects of D23N Iowa mutant amyloid beta-protein. J Biol Chem 276: 32860-32866.
49. Klunk W E, Pettegrew J W, Abraham D J (1989) Quantitative evaluation of congo red binding to amyloid-like proteins with a beta-pleated sheet conformation. J Histochem Cytochem 37: 1273-1281.
50. Klunk W E, Wang Y, Huang G F, Debnath M L, Holt D P, et al. (2001) Uncharged thioflavin-T derivatives bind to amyloid-beta protein with high affinity and readily enter the brain. Life Sci 69: 1471-1484.
51. LeVine H, 3rd (1993) Thioflavine T interaction with synthetic Alzheimer's disease beta-amyloid peptides: detection of amyloid aggregation in solution. Protein Sci 2: 404-410.
52. Puchtler H, Sweat F, Levine M (1962) On the binding of Congo red by amyloid. J Histochem Cytochem 10: 355-364.
53. Agdeppa E D, Kepe V, Petri A, Satyamurthy N, Liu J, et al. (2003) In vitro detection of (S)-naproxen and ibuprofen binding to plaques in the Alzheimer's brain using the positron emission tomography molecular imaging probe 2-(1-{6-[(2-[18F]fluoroethyl)(methyl)amino]-2-naphthyl}ethylidene)malononitrile. Neuroscience 117: 723-730.
54. Smid L M, Vovko T D, Popovic M, Petric A, Kepe V, et al. (2006) The 2,6-disubstituted naphthalene derivative FDDNP labeling reliably predicts Congo red birefringence of protein deposits in brain sections of selected human neurodegenerative diseases. Brain Pathol 16: 124-130.
55. Eilers H, Niemann C (2003) Clinically important drug interactions with intravenous anaesthetics in older patients. Drugs Aging 20: 969-980.
56. Meinhardt J, Sachse C, Hortschansky P, Grigorieff N, Fandrich M (2009) Abeta(1-40) fibril polymorphism implies diverse interaction patterns in amyloid fibrils. J Mol Biol 386: 869-877.
57. Andrews M E, Inayathullah N M, Jayakumar R, Malar E J (2009) Conformational polymorphism and cellular toxicity of IAPP and beta AP domains. J Struct Biol 166: 116-125.
58. Petric A, Jacobson A F, Barrio J R (1998) Functionalization of a viscosity-sensitive fluorophore for probing of biological systems. Bioorg Med Chem Let 8: 1455-1460.
59. McCoy A J, Grosse-Kunstleve R W, Adams P D, Winn M D, Storoni L C, et al. (2007) Phaser crystallographic software. J Appl Cryst 40: 658-674.
60. Murshudov G N, Vagin A A, Dodson E J (1997) Refinement of macromolecular structures by the maximum-likelihood method. Acta Crystallogr D Biol Crystallogr 53: 240-255.
61. Emsley P, Cowtan K (2004) Coot: model-building tools for molecular graphics. Acta Crystallogr D Biol Crystallogr 60: 2126-2132.
62. DeLano W L (2002) DeLano Scientific, San Carlos, Calif., USA.
63. Brunger A T, Adams P D, Clore G M, DeLano W L, Gros P, et al. (1998) Crystallography & NMR system: a new software suite for macromolecular structure determination. Acta Crystallogr D Biol Crystallogr 54: 905-921.
64. Brunger A T (2007) Version 1.2 of the crystallography and NMR system. Nat Protoc 2: 2728-2733.
65. Bostrom J, Greenwood J R, Gottfries J (2003) Assessing the performance of OMEGA with respect to retrieving bioactive conformations. J Mol Graph Model 21: 449-462.
66. Davis I W, Baker D (2009) RosettaLigand docking with full ligand and receptor flexibility. J Mol Biol 385: 381-392.
67. Meiler J, Baker D (2006) ROSETTALIGAND: protein-small molecule docking with full side-chain flexibility. Proteins 65: 538-548.
68. Brunger A T (1992) Free R value: a novel statistical quantity for assessing the accuracy of crystal structures. Nature 355: 472-475.
69. Lee B, Richards F M (1971) The interpretation of protein structures: estimation of static accessibility. J Mol Biol 55: 379-400.
70. Saff E B, Kuijlaars A B J (1997) Distributing many points on a sphere. Math Intelligencer 19: 5-11.
71. Eisenberg D, Wesson M, Yamashita M (1989) Interpretation of protein folding and binding with atomic solvation parameters. chemica Scripta 29A: 217-221.
72. Linden A, Mayans O, Meyer-Klaucke W, Antranikian G, Wilmanns M (2003) Differential regulation of a hyperthermophilic alpha-amylase with a novel (Ca,Zn) two-metal center by zinc. J Biol Chem 278: 9875-9884.

### Example II

### Supporting Information

### A. Using Computational Docking for Structure Determination

In all four structures reported in Example I, the electron density attributed to the small molecule was undifferentiated (to different extents), which hindered the determination of the structures in atomic detail. After assignment of the peptide segment into the electron density 2Fo-Fc map, the difference Fo-Fc map showed positive density that resembled a narrow and long tube running along the fiber (see e.g. in FIGS. 6 and 11). This density indicated the binding of the small molecule, and yet was insufficiently detailed for the atomic assignment of the small molecule. It is noteworthy that the small molecule constitutes a significant part of the asymmetric unit of the crystal in the complexes of small molecules with the peptide segments. For example, in the complex of KLVFFA (SEQ ID NO: 1) with orange-G, the number of atoms of orange-G molecules constitutes ˜20% of the total atoms in the asymmetric unit. Therefore, we anticipated that computational docking [1,2] (Methods) would allow for the correct assignment of the small molecule atoms.

The generated docked structures were refined and evaluated based on their free-R value [3] (Methods). In the case of KLVFFA (SEQ ID NO: 1) or VQIVYK (SEQ ID NO: 2) with orange-G, the crystallographic refinement in the presence of the small molecule significantly decreased the free-R value (by 5% and 2%, respectively). In the case of VQIVYK (SEQ ID NO: 2) with DDNP or curcumin, the refinement with the small molecule did not improve the free-R value and we concluded that the x-ray diffraction does not allow the determination of the position of the small molecule in atomic detail.

### Incommensurate Structures

In the three structures with VQIVYK (SEQ ID NO: 2) complexed with orange-G, DDNP and curcumin, the lengths of the small molecules (DDNP ˜12×5 Å, curcumin ˜19×5 Å and orange-G ˜9.5×8 Å) span multiple unit cells of the fibril (4.8-4.9 Å along the fiber axis; FIGS. 4 and 6); that is, the dimensions of the small molecule and the fibril unit cell were incommensurate [4,5]. Without wishing to be bound by any particular mechanism, it is suggested that the small molecule is drifting along the fiber axis, leading to disorder along one dimension (that of the fiber axis).

Based on our structures we extrapolate that apolar compounds, such as DDNP and curcumin, bind to cylindrical cavities formed between pairs of β-sheets in amyloid structures. These cavities are frequently surrounded by hydrophobic and aromatic side chains [6,7], forming a binding motif for poly-aromatic compounds often reported to affect fibrillation [7-14]. Nevertheless, the binding is insufficiently specific, such that the molecules can be situated with different spacing along the fiber. Moreover, since the main constraint on binding is the width of the cylindrical cavity, the small molecule can not only drift along the fiber, but also rotate along its long dimension, and flip 180° perpendicular to its long dimension. In the crystalline form, these degrees of freedom will lead to crystal disorder along the fiber axis, as we see here for the DDNP and curcumin complexes.

The binding of orange-G to the fibers is more specific than the binding of apolar compounds, via salt links between the negatively charged sulfonic acid groups of orange-G and the lysine side chains (FIGS. 1 and 4). Congruently, the mass spectrometric analyses of the crystals showed that the molar abundance of orange-G in the crystals is high (˜1:1 and ˜1:10 stoichiometries with KLVFFA (SEQ ID NO: 1) and VQIVYK (SEQ ID NO: 2), respectively) in comparison to the low molar abundance of curcumin and DDNP (˜1:100 and ˜1:400 stoichiometries with VQIVYK (SEQ ID NO: 2), respectively). Indeed, the structures of orange-G complexed with both KLVFFA (SEQ ID NO: 1) and VQIVYK (SEQ ID NO: 2) were more ordered, and the determination of the position of the orange-G in atomic detail was enabled using crystallographic refinements coupled with computational docking [1,2]. On the other hand, the low occupancy of DDNP and curcumin in the crystals, coupled with their possible drifting and rotation, corresponds to the disorder seen in the electron density map attributed to the small molecules, which prevented the determination of their position in atomic detail.

The high abundance of orange-G in the KLVFFA (SEQ ID NO: 1) fiber corresponds to the detailed electron density for orange-G obtained following the computational docking (FIG. 12A-C). This electron density was validated via a simulated annealing composite omit map (FIG. 12D-F). In this structure, the KLVFFA (SEQ ID NO: 1) segment forms β-strands that are packed in an antiparallel orientation, associating to a unit cell dimension of 9.54 Å along the fiber axis, which is sufficiently long to accommodate the orange-G (FIG. 1). Furthermore, we observed high complementarity between the chemical features of orange-G and the binding cavity on the KLVFFA (SEQ ID NO: 1) fiber. The KLVFFA (SEQ ID NO: 1) segment is a stretch of apolar side-chains preceded by a positively charged N-terminus. The apolar stretch, which includes aromatic side chains, attracts the aromatic rings of orange-G, while the lysine ammonium ions satisfy their charge by forming salt links to the negatively charged sulfate ions of orange-G (FIGS. 1-2 and 9). In the complex with VQIVYK (SEQ ID NO: 2), the sulfate ions of orange-G again form a polar network of interactions (FIG. 4). However, the binding cavity of orange-G within the VQIVYK (SEQ ID NO: 2) fibers is only 40% hydrophobic vs. the 80% hydrophobic cavity within the KLVFFA (SEQ ID NO: 1) complex (FIGS. 2 and 5). In the VQIVYK (SEQ ID NO: 2) fibers, the aromatic rings of orange-G are packed against the hydrophobic side chains of Val4 and the carbon chain of Lys6, as well as against the polar side chain of Gln2 (FIG. 5). The differences in the binding cavities within the VQIVYK (SEQ ID NO: 2) and KLVFFA (SEQ ID NO: 1) structures may be responsible for the lower molecular abundance of orange-G in the VQIVYK (SEQ ID NO: 2) co-crystals, to the incommensurate fiber unit cell length, and to the resultant partial electron density observed for orange-G (FIGS. 12-13).

### REFERENCES FOR EXAMPLE II

1. Davis I W, Baker D (2009) RosettaLigand docking with full ligand and receptor flexibility. J Mol Biol 385: 381-392.
2. Meiler J, Baker D (2006) ROSETTALIGAND: protein-small molecule docking with full side-chain flexibility. Proteins 65: 538-548.
3. Brunger A T (1992) Free R value: a novel statistical quantity for assessing the accuracy of crystal structures. Nature 355: 472-475.
4. de Wolff P M (1974) The Pseudo-Symmetry of Modulated Crystal Structures. Acta Crystallogr A 30: 777-785.
5. Hao Q, Liu Yw, Fan Hf (1987) Direct methods in superspace. I. Preliminary theory and test on the determination of incommensurate modulated structures. Acta Crystallogr A 43: 820-824.
6. Gazit E (2002) A possible role for pi-stacking in the self-assembly of amyloid fibrils. FASEB J 16: 77-83.
7. Porat Y, Abramowitz A, Gazit E (2006) Inhibition of amyloid fibril formation by polyphenols: structural similarity and aromatic interactions as a common inhibition mechanism. Chem Biol Drug Des 67: 27-37.
8. Porat Y, Mazor Y, Efrat S, Gazit E (2004) Inhibition of islet amyloid polypeptide fibril formation: a potential role for heteroaromatic interactions. Biochemistry 43: 14454-14462.
9. Ferrao-Gonzales A D, Robbs B K, Moreau V H, Ferreira A, Juliano L, et al. (2005) Controlling {beta}-Amyloid Oligomerization by the Use of Naphthalene Sulfonates: trapping low molecular weight oligomeric species. J Biol Chem 280: 34747-34754.
10. Necula M, Breydo L, Milton S, Kayed R, van der Veer W E, et al. (2007) Methylene blue inhibits amyloid Abeta oligomerization by promoting fibrillization. Biochemistry 46: 8850-8860.
11. Necula M, Kayed R, Milton S, Glabe C G (2007) Small molecule inhibitors of aggregation indicate that amyloid beta oligomerization and fibrillization pathways are independent and distinct. J Biol Chem 282: 10311-10324.
12. Ono K, Yoshiike Y, Takashima A, Hasegawa K, Naiki H, et al. (2003) Potent anti-amyloidogenic and fibril-destabilizing effects of polyphenols in vitro: implications for the prevention and therapeutics of Alzheimer's disease. J Neurochem 87: 172-181.
13. Yang F, Lim G P, Begum A N, Ubeda O J, Simmons M R, et al. (2005) Curcumin inhibits formation of amyloid beta oligomers and fibrils, binds plaques, and reduces amyloid in vivo. J Biol Chem 280: 5892-5901.
14. Cohen T, Frydman-Marom A, Rechter M, Gazit E (2006). Inhibition of amyloid fibril formation and cytotoxicity by hydroxyindole derivatives. Biochemistry 45: 4727-4735.
15. Choi Y T, Jung C H, Lee S R, Bae J H, Baek W K, et al. (2001) The green tea polyphenol (−)-epigallocatechin gallate attenuates beta-amyloid-induced neurotoxicity in cultured hippocampal neurons. Life Sci 70: 603-614.
16. Mathis C A, Bacskai B J, Kajdasz S T, McLellan M E, Frosch M P, et al. (2002) A lipophilic thioflavin-T derivative for positron emission tomography (PET) imaging of amyloid in brain. Bioorg Med Chem Let 12: 295-298.
17. Agdeppa E D, Kepe V, Liu J, Small G W, Huang S C, et al. (2003) 2-Dialkylamino-6-acylmalononitrile substituted naphthalenes (DDNP analogs): novel diagnostic and therapeutic tools in Alzheimer's disease. Mol Imaging Biol 5: 404-417.
18. Jacobson A, Petric A, Hogenkamp D, Sinur A, Barrio J R (1996) 1,1-Dicyano-2-[6-(dimethylamino)naphthalen-2-yl]propene (DDNP): A Solvent Polarity and Viscosity Sensitive Fluorophore for Fluorescence Microscopy. J Am Chem Soc 118: 5572-5579.
19. Shoghi-Jadid K, Small G W, Agdeppa E D, Kepe V, Ercoli L M, et al. (2002) Localization of neurofibrillary tangles and beta-amyloid plaques in the brains of living patients with Alzheimer disease. Am J Geriatr Psychiatry 10: 24-35.
20. Smid L M, Vovko T D, Popovic M, Petric A, Kepe V, et al. (2006) The 2,6-disubstituted naphthalene derivative FDDNP labeling reliably predicts Congo red birefringence of protein deposits in brain sections of selected human neurodegenerative diseases. Brain Pathol 16: 124-130.
   Table 1. Screening for Co-Crystals from Mixtures of Amyloid-Like Segments with Small Molecules.

We choose small molecules that were reported to affect fibrillation of different amyloid-forming proteins [10,11,13], including natural compounds [15], a Thioflavin derivative: Pittsburgh compound B (PIB) [16], as well as a molecule that constitutes half of the curcumin molecule: (−)-2-Methoxy-4-methylphenol (Creosol). We also screened for complexes with biological marker that detect amyloid fibers in-vivo, developed and synthesized by Jorge R. Barrio and co-workers [17-20].

We used 34 different small-molecules combined with different amyloid-like segments to generate an overall of 89 different mixtures. We note that several different molecular ratios (ranging between 1:1 and 1:10 small-molecule:segment) were tested (details are not specified in the table) resulting in >100 different co-crystallization trials. Each mixture was screened for the formation of co-crystals with 768 different crystallization conditions. In many cases, crystals grown from various conditions were tested (details are not specified in the table). We note that soaking experiments (adding the small molecule after growing crystals from the amyloid-like segment alone), tested for several of the different combinations, failed to show the presence of the small molecule. This is expected due to the lack of solvent channels in the crystal packing of the amyloid-like segments.

From the 89 mixtures detailed in the Table, 4 structures of complexes were determined. 14 mixtures did not show formation of crystals in the conditions tested over several months. 47 mixtures resulted in fibrous or colorless crystals that were not tested, or crystals with too poor x-ray diffraction to be determined. Crystals grown from 21 mixtures showed the presence of only the amyloid-like segment, while 3 showed the presence of only the small molecule.

TABLE 1
Screening for co-crystals from mixtures of amyloid-like segments with small molecules

| Small Molecule | Amyloid-like segment | Co-crystallization result |
|---|---|---|
| Orange G | VQIVYK from tau (SEQ ID NO: 2) | Structure of the complex was |
| | | determined (FIG. 3). |
| | KLVFFA (residues 16-21) from Aβ (SEQ | Structure of the complex was |
| | ID NO: 1) | determined (FIG. 1). |
| | KLVFFG (residues 16-21) - | Crystals with too poor x-ray |
| | Flemish (A21G) mutation from Aβ (SEQ | diffraction to be determined. |
| | ID NO: 3) | |
| | KLVFFAK (residues 16-22) - | No crystals. |
| | Italian (E22K) mutation from Aβ (SEQ | |
| | ID NO: 4) | |
| | KLVFFAG (residues 16-22) - | Fibrous crystals. |
| | Artic (E22G) mutation from Aβ (SEQ ID | |
| | NO: 5) | |
| | KLVFFAEN (residues 16-23) - | Crystals with too poor x-ray |
| | Iowa (D23N) mutations from Aβ (SEQ ID | diffraction to be determined. |
| | NO: 6) | |
| | KLVFFAENVG (residues 16-25) - | No crystals. |
| | Iowa (D23N) mutations from Aβ (SEQ ID | |
| | NO: 7) | |
| | KLVFFAGNVGSNK (residues 16-28) - | No crystals. |
| | Artic (E22G) and Iowa (D23N) mutations | |
| | from Aβ (SEQ ID NO: 8) | |
| | GDVGSNK (residues 22-28) - | No crystals. |
| | Artic (E22G) mutation from Aβ (SEQ ID | |
| | NO: 9) | |
| | QDVGSNK (residues 22-28) - | No crystals. |
| | Dutch (E22Q) mutation from Aβ (SEQ | |
| | ID NO: 10) | |
| | GGVVIA (residues 37-42) from Aβ (SEQ | Crystals with too poor x-ray |
| | ID NO: 11) | diffraction to be determined. |
| | LVFFAEDVGSNKGAI IGLMVGGVV | No crystals. |
| | (residues 17-40) from Aβ (SEQ ID | |
| | NO: 12) | |
| | LVFFAEDVGSNKGAI | Fibrous crystals. |
| | IGLMVGGVVIA (residues 17-42) from | |
| | Aβ (SEQ ID NO: 13) | |
| | GVVEVD (residues 734-739) from Aβ A4 | Structure determined and |
| | protein (APP) (SEQ ID NO: 14) | showed the presence of only |
| | | orange-G. |
| | GDVIEV from α-crystalline (SEQ ID | Crystals with too poor x-ray |
| | NO: 15) | diffraction to be determined. |
| | SSTNVG from amylin (SEQ ID NO: 16) | All crystals formed (under |
| | | various crystallization |
| | | conditions) were colorless and |
| | | were not tested further. |
| | GNNQQNY from yeast prion protein | All crystals formed (under |
| | Sup35 (SEQ ID NO: 17) | various crystallization |
| | | conditions) were colorless and |
| | | were not tested further. |
| Curcumin | VQIVYK from tau (SEQ ID NO: 2) | Structure of the complex was |
| | | determined (FIG. 4). |
| | KLVFFA (residues 16-21) from Aβ (SEQ | Crystals with too poor x-ray |
| | ID NO: 1) | diffraction to be determined. |
| | GGVVIA (residues 37-42) from Aβ (SEQ | Structure determined and |
| | ID NO: 11) | showed the presence of only |
| | | GGVVIA (SEQ ID NO: 11). |
| | GVVEVD (residues 734-739) from Aβ A4 | Crystals with too poor x-ray |
| | protein (APP) (SEQ ID NO: 14) | diffraction to be determined. |
| | GDVIEV from α-crystalline (SEQ ID NO: | Crystals with too poor x-ray |
| | 15) | diffraction to be determined. |
| | SSTNVG from amylin (SEQ ID NO: 16) | Structure determined and |
| | | showed the presence of just |
| | | SSTNVG (SEQ ID NO: 16). |
| Phenol Red | VQIVYK from tau (SEQ ID NO: 2) | Crystals with too poor x-ray |
| | | diffraction to be determined. |
| | GGVVIA (residues 37-42) from Aβ (SEQ | Structure determined and |
| | ID NO: 11) | showed the presence of only |
| | | GGVVIA (SEQ ID NO: 11). |
| | SSTNVG from amylin (SEQ ID NO: 16) | Crystals with too poor x-ray |
| | | diffraction to be determined. |
| | NFGAILSS (residues 22-29) from amylin | No crystals. |
| | (SEQ ID NO: 18) | |
| | SSNNFGAILSS (residues 19-29) from | No crystals. |
| | amylin (SEQ ID NO: 19) | |
| | SNNFGAILSS (residues 20-29) from | Crystals with too poor x-ray |
| | amylin (SEQ ID NO: 20) | diffraction to be determined. |
| Thiofavin T | VQIVYK from tau (SEQ ID NO: 2) | Structure determined and |
| | | showed the presence of only |
| | | Thiofavin T. |
| | KLVFFA (residues 16-21) from Aβ (SEQ | Structure determined and |
| | ID NO: 1) | showed the presence of just |
| | | KLVFFA (SEQ ID NO: 1). |
| | GGVVIA (residues 37-42) from Aβ (SEQ | Crystals with too poor x-ray |
| | ID NO: 11) | diffraction to be determined. |
| | GVVEVD (residues 734-739) from Aβ A4 | Structure determined and |
| | protein (APP) (SEQ ID NO: 14) | showed the presence of just |
| | | GVVEVD (SEQ ID NO: 14) in |
| | | a unique anti-parallel packing. |
| | GDVIEV from α-crystalline (SEQ ID NO: | Crystals with too poor x-ray |
| | 15) | diffraction to be determined. |
| Chicago sky blue | VQIVYK from tau (SEQ ID NO: 2) | Crystals with too poor x-ray |
| 6B | | diffraction to be determined. |
| | GGVVIA (residues 37-42) from Aβ (SEQ | Structure determined and |
| | ID NO: 11) | showed the presence of only |
| | | GGVVIA (SEQ ID NO: 11). |
| Rhodamine B | VQIVYK from tau (SEQ ID NO: 2) | Structure determined and |
| | | showed the presence of only |
| | | VQIVYK (SEQ ID NO: 2). |
| | GGVVIA (residues 37-42) from Aβ (SEQ | Crystals with too poor x-ray |
| | ID NO: 11) | diffraction to be determined. |
| Azure C | VQIVYK from tau (SEQ ID NO: 2) | Crystals with too poor x-ray |
| | | diffraction to be determined. |
| | GGVVIA (residues 37-42) from Aβ (SEQ | Crystals with too poor x-ray |
| | ID NO: 11) | diffraction to be determined. |
| Rolitetracycline | VQIVYK from tau (SEQ ID NO: 2) | Crystals with too poor x-ray |
| | | diffraction to be determined. |
| | GGVVIA (residues 37-42) from Aβ (SEQ | Crystals with too poor x-ray |
| | ID NO: 11) | diffraction to be determined. |
| Myristyltrimethyl- | VQIVYK from tau (SEQ ID NO: 2) | No crystals. |
| ammonium | GGVVIA (residues 37-42) from Aβ (SEQ | Crystals with too poor x-ray |
| bromide | ID NO: 11) | diffraction to be determined. |
| o-vanillin | VQIVYK from tau (SEQ ID NO: 2) | Crystals with too poor x-ray |
| | | diffraction to be determined. |
| | GGVVIA (residues 37-42) from Aβ (SEQ | Crystals with too poor x-ray |
| | ID NO: 11) | diffraction to be determined. |
| Juglone | VQIVYK from tau (SEQ ID NO: 2) | Structure determined and |
| | | showed the presence of only |
| | | VQIVYK (SEQ ID NO: 2). |
| | GGVVIA (residues 37-42) from Aβ (SEQ | Structure determined and |
| | ID NO: 11) | showed the presence of only |
| | | GGVVIA (SEQ ID NO: 11). |
| Hexadecyltrimethyl | VQIVYK from tau (SEQ ID NO: 2) | Crystals with too poor x-ray |
| ammonium | | diffraction to be determined. |
| bromide | GGVVIA (residues 37-42) from Aβ (SEQ | Crystals with too poor x-ray |
| | ID NO: 11) | diffraction to be determined. |
| 1,2- | VQIVYK from tau (SEQ ID NO: 2) | Crystals with too poor x-ray |
| Naphthoquinone | | diffraction to be determined. |
| | GGVVIA (residues 37-42) from Aβ (SEQ | Crystals with too poor x-ray |
| | ID NO: 11) | diffraction to be determined. |
| Lacmoid | VQIVYK from tau (SEQ ID NO: 2) | No crystals. |
| | GGVVIA (residues 37-42) from Aβ (SEQ | Fibrous crystals. |
| | ID NO: 11) | |
| Perphenazine | VQIVYK from tau (SEQ ID NO: 2) | Structure determined and |
| | | showed the presence of only |
| | | VQIVYK (SEQ ID NO: 2). |
| Thiofavin S | VQIVYK from tau (SEQ ID NO: 2) | Structure determined and |
| | | showed the presence of only |
| | | VQIVYK (SEQ ID NO: 2). |
| Rifamycin SV | GGVVIA (residues 37-42) from Aβ (SEQ | Crystals with too poor x-ray |
| sod. Salt | ID NO: 11) | diffraction to be determined. |
| | SSTNVG from amylin (SEQ ID NO: 16) | Structure determined and |
| | | showed the presence of just |
| | | SSTNVG (SEQ ID NO: 16). |
| Meclocycline | VQIVYK from tau (SEQ ID NO: 2) | Crystals with too poor x-ray |
| sulfosalicylate salt | | diffraction to be determined. |
| Eosin Y | VQIVYK from tau (SEQ ID NO: 2) | Fibrous crystals. |
| | GGVVIA (residues 37-42) from Aβ (SEQ | Crystals with too poor x-ray |
| | ID NO: 11) | diffraction to be determined. |
| 2,2′- | GGVVIA (residues 37-42) from Aβ (SEQ | Structure determined and |
| Dihydroxybenzophenone | ID NO: 11) | showed the presence of only |
| | | GGVVIA (SEQ ID NO: 11). |
| Methylene Blue | VQIVYK from tau (SEQ ID NO: 2) | Structure determined and |
| | | showed the presence of only |
| | | Methylene Blue. |
| | GGVVIA (residues 37-42) from Aβ (SEQ | Crystals with too poor x-ray |
| | ID NO: 11) | diffraction to be determined. |
| Benserazide | VQIVYK from tau (SEQ ID NO: 2) | Fibrous crystals. |
| hydrochloride | GGVVIA (residues 37-42) from Aβ (SEQ | Structure determined and |
| | ID NO: 11) | showed the presence of only |
| | | GGVVIA (SEQ ID NO: 11). |
| 2-Methoxy-4- | VQIVYK from tau (SEQ ID NO: 2) | Structure determined and |
| methylphenol | | showed the presence of only |
| (Creosol) | | VQIVYK (SEQ ID NO: 2). |
| | GGVVIA (residues 37-42) from Aβ (SEQ | Crystals with too poor x-ray |
| | ID NO: 11) | diffraction to be determined. |
| R-(−)- | VQIVYK from tau (SEQ ID NO: 2) | Crystals with too poor x-ray |
| Apomorphine | | diffraction to be determined. |
| hydrochloride | GGVVIA (residues 37-42) from Aβ (SEQ | Structure determined and |
| hemihydrate | ID NO: 11) | showed the presence of only |
| | | GGVVIA (SEQ ID NO: 11). |
| Dobutamine | VQIVYK from tau (SEQ ID NO: 2) | Crystals with too poor x-ray |
| hydrochloride | | diffraction to be determined. |
| Neocuproine | VQIVYK from tau (SEQ ID NO: 2) | Structure determined and |
| | | showed the presence of only |
| | | VQIVYK (SEQ ID NO: 2). |
| (−)- | VQIVYK from tau (SEQ ID NO: 2) | No crystals. |
| Epigallocatechin | GGVVIA (residues 37-42) from Aβ (SEQ | Structure determined and |
| gallate | ID NO: 11) | showed the presence of only |
| | | GGVVIA (SEQ ID NO: 11) |
| Epicatechin | VQIVYK from tau (SEQ ID NO: 2) | No crystals. |
| PIB | VQIVYK from tau (SEQ ID NO: 2) | Structure determined and |
| | | showed the presence of just |
| | | VQIVYK (SEQ ID NO: 2). |
| | KLVFFA (residues 16-21) from Aβ (SEQ | Crystals with too poor x-ray |
| | ID NO: 1) | diffraction to be determined. |
| DDNP | VQIVYK from tau (SEQ ID NO: 2) | Structure of the complex was |
| | | determined (FIG. 4). |
| | KLVFFA (residues 16-21) from Aβ (SEQ | Crystals with too poor x-ray |
| | ID NO: 1) | diffraction to be determined. |
| | KLVFFG (residues 16-21) -Flemish | Crystals with too poor x-ray |
| | (A21G) mutation from Aβ (SEQ ID NO: | diffraction to be determined. |
| | 3) | |
| | KLVFFAK (residues 16-22) - Italian | No crystals. |
| | (E22K) mutation from Aβ (SEQ ID NO: | |
| | 4) | |
| FDDNP | VQIVYK from tau (SEQ ID NO: 2) | Crystals with too poor x-ray |
| | | diffraction to be determined. |
| | KLVFFA (residues 16-21) from Aβ (SEQ | Structure determined and |
| | ID NO: 1) | showed the presence of just |
| | | KLVFFA (SEQ ID NO: 1) in a |
| | | unique packing. |
| | LVFFAEDVGSNKGAI IGLMVGGVV | Fibrous crystals with no x-ray |
| | (residues 17-40) from Aβ (SEQ ID NO: | diffraction. |
| | 12) | |
| | LVFFAEDVGSNKGAIIGLMVGGVVIA | Crystals with too poor x-ray |
| | (residues 17-42) from Aβ (SEQ ID NO: | diffraction to be determined. |
| | 13) | |
| CFDDNP | KLVFFG (residues 16-21)-Flemish | Crystals with too poor x-ray |
| | (A21G) mutation from Aβ (SEQ ID NO: | diffraction to be determined. |
| | 3) | |
| | KLVFFAK (residues 16-22) - Italian | No crystals. |
| | (E22K) mutation from Aβ (SEQ ID NO: | |
| | 4) | |
| AZET | VQIVYK from tau (SEQ ID NO: 2) | Structure determined and |
| | | showed the presence of just |
| | | VQIVYK (SEQ ID NO: 2). |
| EB-I-68 | KLVFFA (residues 16-21) from Aβ (SEQ | Fibrous crystals. |
| | ID NO: 1) | |

TABLE 2
Data collection and refinement statistics (molecular replacement).

| | KLVFFA (SEQ ID NO: 1)- | VQIVYK (SEQ ID NO: 2)- |
|---|---|---|
| | orange-G | orange-G |
| PDB accession code | 3OVJ | 3OVL |

Data Collection

| | | |
|---|---|---|
| Beamline | APS 24-ID-E | APS 24-ID-E |
| Date | 2008 Nov. 15 and 2009 Nov. 13 | 2008 Nov. 17 |
| Space group | P 1 | C 2 |
| Cell dimensions: | | |
| a, b, c (Å) | 9.54, 26.01, 25.80 | 55.06, 4.83, 22.13 |
| α, β, γ (°) | 62.3, 88.6, 88.5 | 90.0, 103.0, 90.0 |
| Resolution range (Å)⁽*⁾ | 26.01-1.8 (1.9-1.8) | 55.06-1.8 (1.9-1.8) |
| R_{merge (square)} (%)⁽*^{)(a)} | 18.9 (42.3) | 17.9 (48.9) |
| I/σI(*) | 4.38 (1.87) | 3.55 (1.20) |
| Completeness (%)⁽*^{)(b)} | 91.5 (71.7) | 87.6 (71.0) |
| Redundancy⁽*⁾ | 3.8 (1.7) | 2.4 (1.4) |
| Unique Reflections | 1870 | 587 |

Refinement

| | | |
|---|---|---|
| Resolution range (Å)⁽*⁾ | 23.0-1.8 (2.0-1.8) | 26.8-1.8 (2.0-1.8) |
| Unique Reflections | 1692 | 525 |
| R_{work}(%)⁽*^{)(c)}/ R_{free} (%)⁽*^{)(d) (e)} | 20.5 (28.9)/22.0 (36.6) | 25.9 (37.7)/26.0 (44.8) |
| Completeness (%)⁽*^{)(b)} | 92.9 (79.9) | 88.0 (76.4) |
| Observations/parameters ratio | 1.6 | 1.5 |
| Test set size [%], selection | 9.2, random | 10.4, random |
| Number of atoms in asymm. unit | 273 | 88 |
| Protein | 208 | 53 |
| Ligand | 54 | 33 |
| Water | 11 | 2 |
| Average B-factor (Å²): | | |
| Average B factor for | 12.0 | 12.4 |
| mainchain atoms | | |
| Average B factor for | 16.1 | 14.7 |
| sidechain atoms | | |
| Average B factor for | 23.3 | 27.1 |
| ligands | | |
| Average B factor for water | 25.4 | 28.4 |
| R.m.s. deviations: | | |
| r.m.s.d. bond length (Å) | 0.01 | 0.01 |
| r.m.s.d. bond angles (°) | 1.69 | 1.22 |
| R.m.s. B: | | |
| R.m.s. B for mainchain | 1.0 | 0.9 |
| atoms | | |
| R.m.s. B for sidechain | 2.7 | 2.9 |
| atoms | | |
| R.m.s. B for ligands | 7.0 | 2.0 |

⁽*⁾Values in brackets are for the highest resolution shells.
^{(a)}Rmerge(square) = Σ(I − I(mean))²/ΣI², where I is the observed intensity of the reflection HKL and the sum is taken over all reflections HKL.
^{(b)}The incompleteness (<80%) of the highest resolution shell is likely due to the rapid decay of the peptide crystals that are naturally susceptible to radiation decay due to their small size.
^{(c)}Rwork = Σ||Fₒ| − |F_{c}||/Σ|Fₒ|.
^{(d)}R_{free} as defined by [3].
^{(e)}The statistical significance of Rfree is limited in the case of peptide structures, where the small size of the unit cell leads to a paucity of reflections. In these cases, the Rfree statistic has proven to be a useful, but less sensitive tool compared with Rfree statistics reported from macromolecular structure.

### REFERENCE

1. Brunger A T (1992) Free R value: a novel statistical quantity for assessing the accuracy of crystal structures. Nature 355:472-475.

TABLE 3
Atomic coordinates of structure of an amyloid-forming peptide KLVFFA (SEQ
ID NO: 1) from amyloid beta in complex with Orange G

| | | |
|---|---|---|
| HEADER | PROTEIN FIBRIL | 16-SEP-10 3OVJ |

| | |
|---|---|
| TITLE | STRUCTURE OF AN AMYLOID FORMING PEPTIDE KLVFFA FROM AMYLOID BETA IN |

| | | |
|---|---|---|
| TITLE | 2 | COMPLEX WITH ORANGE G |

| | |
|---|---|
| COMPND | MOL_ID: 1; |

| | | |
|---|---|---|
| COMPND | 2 | MOLECULE: KLVFFA HEXAPEPTIDE SEGMENT FROM AMYLOID BETA; |
| COMPND | 3 | CHAIN: A, B, C, D; |
| COMPND | 4 | FRAGMENT: KLVFFA (UNP RESIDUES 687-692); |
| COMPND | 5 | ENGINEERED: YES |

| | |
|---|---|
| SOURCE | MOL_ID: 1; |

| | | |
|---|---|---|
| SOURCE | 2 | SYNTHETIC: YES; |
| SOURCE | 3 | ORGANISM_TAXID: 32630; |
| SOURCE | 4 | OTHER_DETAILS: KLVFFA (RESIDUES 16-21) FROM AMYLOID BETA, |
| SOURCE | 5 | SYNTHESIZED |

| | |
|---|---|
| KEYWDS | AMYLOID-LIKE PROTOFIBRIL, PROTEIN FIBRIL |
| EXPDTA | X-RAY DIFFRACTION |
| AUTHOR | M. LANDAU, D. EISENBERG |

| | | |
|---|---|---|
| REVDAT | 1 | 06-JUL-11 3OVJ 0 |

| JRNL | AUTH | | M. LANDAU, M. R. SAWAYA, K. F. FAULL, A. LAGANOWSKY, L. JIANG, | |
|---|---|---|---|---|
| JRNL | AUTH | 2 | S. A. SIEVERS, J. LIU, J. R. BARRIO, D. EISENBERG | |
| JRNL | TITL | | TOWARDS A PHARMACOPHORE FOR AMYLOID. | |
| JRNL | REF | | PLOS BIOL. | V. 9 E1001 2011 |

| | | |
|---|---|---|
| JRNL | REFN | ISSN 1544-9173 |

| | | |
|---|---|---|
| JRNL | PMID | 21695112 |
| JRNL | DOI | 10.1371/JOURNAL.PBIO.1001080 |

| | | | |
|---|---|---|---|
| REMARK | 2 | | |
| REMARK | 2 | RESOLUTION. | 1.80 ANGSTROMS. |
| REMARK | 3 | | |
| REMARK | 3 | REFINEMENT. | |
| REMARK | 3 | PROGRAM | : REFMAC 5.5.0109 |
| REMARK | 3 | AUTHORS | : MURSHUDOV, VAGIN, DODSON |
| REMARK | 3 | | |

| REMARK | 3 | REFINEMENT TARGET: MAXIMUM LIKELIHOOD | | |
|---|---|---|---|---|
| REMARK | 3 | | | |
| REMARK | 3 | DATA USED IN REFINEMENT. | | |
| REMARK | 3 | RESOLUTION RANGE HIGH | (ANGSTROMS) | : 1.80 |
| REMARK | 3 | RESOLUTION RANGE LOW | (ANGSTROMS) | : 23.02 |
| REMARK | 3 | DATA CUTOFF | (SIGMA(F)) | : 0.000 |
| REMARK | 3 | COMPLETENESS FOR RANGE | (%) | : 91.9 |

| | | | |
|---|---|---|---|
| REMARK | 3 | NUMBER OF REFLECTIONS | : 1864 |
| REMARK | 3 | | |
| REMARK | 3 | FIT TO DATA USED IN REFINEMENT. | |

| REMARK | 3 | CROSS-VALIDATION METHOD | | : THROUGHOUT |
|---|---|---|---|---|
| REMARK | 3 | FREE R VALUE TEST SET SELECTION | | : RANDOM |
| REMARK | 3 | R VALUE | (WORKING + TEST SET) | : 0.207 |
| REMARK | 3 | R VALUE | (WORKING SET) | : 0.205 |

| REMARK | 3 | FREE R VALUE | | : 0.220 |
|---|---|---|---|---|
| REMARK | 3 | FREE R VALUE TEST SET SIZE | (%) | : 9.200 |

| | | | |
|---|---|---|---|
| REMARK | 3 | FREE R VALUE TEST SET COUNT | : 172 |
| REMARK | 3 | | |
| REMARK | 3 | FIT IN THE HIGHEST RESOLUTION BIN. | |

| REMARK | 3 | TOTAL NUMBER OF BINS USED | | : 5 |
|---|---|---|---|---|
| REMARK | 3 | BIN RESOLUTION RANGE HIGH | (A) | : 1.80 |
| REMARK | 3 | BIN RESOLUTION RANGE LOW | (A) | : 2.02 |

| | | | | |
|---|---|---|---|---|
| REMARK | 3 | REFLECTION IN BIN | (WORKING SET) | : 434 |
| REMARK | 3 | BIN COMPLETENESS | (WORKING + TEST) (%) | : 79.86 |
| REMARK | 3 | BIN R VALUE | (WORKING SET) | : 0.2890 |

| | | | |
|---|---|---|---|
| REMARK | 3 | BIN FREE R VALUE SET COUNT | : 26 |
| REMARK | 3 | BIN FREE R VALUE | : 0.3660 |
| REMARK | 3 | | |

| REMARK | 3 | NUMBER OF NON-HYDROGEN ATOMS USED IN REFINEMENT. | |
|---|---|---|---|
| REMARK | 3 | PROTEIN ATOMS | : 208 |
| REMARK | 3 | NUCLEIC ACID ATOMS | : 0 |
| REMARK | 3 | HETEROGEN ATOMS | : 54 |
| REMARK | 3 | SOLVENT ATOMS | : 11 |
| REMARK | 3 | | |
| REMARK | 3 | B VALUES. | |

| | | | | |
|---|---|---|---|---|
| REMARK | 3 | FROM WILSON PLOT | (A**2) | : 22.60 |
| REMARK | 3 | MEAN B VALUE | (OVERALL, A**2) | : 16.46 |

| REMARK | 3 | OVERALL ANISOTROPIC B VALUE. | | |
|---|---|---|---|---|
| REMARK | 3 | B11 (A**2) : −0.30000 | | |
| REMARK | 3 | B22 (A**2) : −0.10000 | | |
| REMARK | 3 | B33 (A**2) : 0.52000 | | |
| REMARK | 3 | B12 (A**2) : −0.07000 | | |
| REMARK | 3 | B13 (A**2) : 0.03000 | | |
| REMARK | 3 | B23 (A**2) : −0.11000 | | |
| REMARK | 3 | | | |
| REMARK | 3 | ESTIMATED OVERALL COORDINATE ERROR. | | |
| REMARK | 3 | ESU BASED ON R VALUE | (A) | :NULL |
| REMARK | 3 | ESU BASED ON FREE R VALUE | (A) | : 0.169 |
| REMARK | 3 | ESU BASED ON MAXIMUM LIKELIHOOD | (A) | : 0.128 |
| REMARK | 3 | ESU FOR B VALUES BASED ON MAXIMUM LIKELIHOOD | (A**2) | : 4.528 |

| REMARK | 3 | | |
|---|---|---|---|
| REMARK | 3 | CORRELATION COEFFICIENTS. | |
| REMARK | 3 | CORRELATION COEFFICIENT FO-FC | : 0.952 |
| REMARK | 3 | CORRELATION COEFFICIENT FO-FC FREE | : 0.932 |
| REMARK | 3 | | |

| REMARK | 3 | RMS DEVIATIONS FROM IDEAL VALUES | | COUNT | RMS | WEIGHT |
|---|---|---|---|---|---|---|
| REMARK | 3 | BOND LENGTHS REFINED ATOMS | (A): | 270 ; | 0.011; | 0.023 |
| REMARK | 3 | BOND LENGTHS OTHERS | (A): | 166 ; | 0.005 ; | 0.020 |
| REMARK | 3 | BOND ANGLES REFINED ATOMS | (DEGREES): | 368 ; | 1.690 ; | 2.229 |
| REMARK | 3 | BOND ANGLES OTHERS | (DEGREES): | 392 ; | 0.690 ; | 3.000 |
| REMARK | 3 | TORSION ANGLES, PERIOD 1 | (DEGREES): | 20 ; | 6.107 ; | 5.000 |
| REMARK | 3 | TORSION ANGLES, PERIOD 2 | (DEGREES): | 8 ; | 37.050 ; | 20.000 |
| REMARK | 3 | TORSION ANGLES, PERIOD 3 | (DEGREES): | 36 ; | 17.862 ; | 15.000 |
| REMARK | 3 | TORSION ANGLES, PERIOD 4 | (DEGREES): | NULL ; | NULL ; | NULL |
| REMARK | 3 | CHIRAL-CENTER RESTRAINTS | (A**3): | 36 ; | 0.091 ; | 0.200 |

| | | | | | | |
|---|---|---|---|---|---|---|
| REMARK | 3 | GENERAL PLANES REFINED ATOMS | (A): | 262 ; | 0.007 ; | 0.020 |
| REMARK | 3 | GENERAL PLANES OTHERS | (A): | 78 ; | 0.001 ; | 0.020 |
| REMARK | 3 | NON-BONDED CONTACTS REFINED ATOMS | (A): | NULL ; | NULL ; | NULL |
| REMARK | 3 | NON-BONDED CONTACTS OTHERS | (A): | NULL ; | NULL ; | NULL |
| REMARK | 3 | NON-BONDED TORSION REFINED ATOMS | (A): | NULL ; | NULL ; | NULL |
| REMARK | 3 | NON-BONDED TORSION OTHERS | (A): | NULL ; | NULL ; | NULL |
| REMARK | 3 | H-BOND (X . . . Y) REFINED ATOMS | (A): | NULL ; | NULL ; | NULL |
| REMARK | 3 | H-BOND (X . . . Y) OTHERS | (A): | NULL ; | NULL ; | NULL |
| REMARK | 3 | POTENTIAL METAL-ION REFINED ATOMS | (A): | NULL ; | NULL ; | NULL |
| REMARK | 3 | POTENTIAL METAL-ION OTHERS | (A): | NULL ; | NULL ; | NULL |
| REMARK | 3 | SYMMETRY VDW REFINED ATOMS | (A): | NULL ; | NULL ; | NULL |
| REMARK | 3 | SYMMETRY VDW OTHERS | (A): | NULL ; | NULL ; | NULL |
| REMARK | 3 | SYMMETRY H-BOND REFINED ATOMS | (A): | NULL ; | NULL ; | NULL |
| REMARK | 3 | SYMMETRY H-BOND OTHERS | (A): | NULL ; | NULL ; | NULL |
| REMARK | 3 | SYMMETRY METAL-ION REFINED ATOMS | (A): | NULL ; | NULL ; | NULL |
| REMARK | 3 | SYMMETRY METAL-ION OTHERS | (A): | NULL ; | NULL ; | NULL |
| REMARK | 3 | | | | | |

| | | | | | |
|---|---|---|---|---|---|
| REMARK | 3 | ISOTROPIC THERMAL FACTOR RESTRAINTS. | COUNT | RMS | WEIGHT |

| REMARK | 3 | MAIN-CHAIN BOND REFINED ATOMS | (A**2): | 120; | 1.214; | 1.500 |
|---|---|---|---|---|---|---|
| REMARK | 3 | MAIN-CHAIN BOND OTHER ATOMS | (A**2): | 44; | 0.260; | 1.500 |
| REMARK | 3 | MAIN-CHAIN ANGLE REFINED ATOMS | (A**2): | 188; | 2.110; | 2.000 |
| REMARK | 3 | SIDE-CHAIN BOND REFINED ATOMS | (A**2): | 150; | 3.191; | 3.000 |
| REMARK | 3 | SIDE-CHAIN ANGLE REFINED ATOMS | (A**2): | 180; | 5.097; | 4.500 |
| REMARK | 3 | | | | | |
| REMARK | 3 | ANISOTROPIC THERMAL FACTOR RESTRAINTS. | | COUNT | RMS | WEIGHT |
| REMARK | 3 | RIGID-BOND RESTRAINTS | (A**2): | NULL ; | NULL ; | NULL |
| REMARK | 3 | SPHERICITY; FREE ATOMS | (A**2): | NULL ; | NULL ; | NULL |
| REMARK | 3 | SPHERICITY; BONDED ATOMS | (A**2): | NULL ; | NULL ; | NULL |

| REMARK | 3 | | |
|---|---|---|---|
| REMARK | 3 | NCS RESTRAINTS STATISTICS | |
| REMARK | 3 | NUMBER OF DIFFERENT NCS GROUPS : NULL | |
| REMARK | 3 | | |
| REMARK | 3 | TLS DETAILS | |
| REMARK | 3 | NUMBER OF TLS GROUPS : NULL | |
| REMARK | 3 | | |
| REMARK | 3 | BULK SOLVENT MODELLING. | |
| REMARK | 3 | METHOD USED : MASK | |
| REMARK | 3 | PARAMETERS FOR MASK CALCULATION | |
| REMARK | 3 | VDW PROBE RADIUS | : 1.40 |
| REMARK | 3 | ION PROBE RADIUS | : 0.80 |
| REMARK | 3 | SHRINKAGE RADIUS | : 0.80 |

| REMARK | 3 | | | |
|---|---|---|---|---|
| REMARK | 3 | OTHER REFINEMENT REMARKS: HYDROGENS HAVE BEEN ADDED IN THE RIDING | | |
| REMARK | 3 | POSITIONS U VALUES: REFINED INDIVIDUALLY | | |
| REMARK | 4 | | | |
| REMARK | 4 | 3OVJ COMPLIES WITH FORMAT V. 3.20, 01-DEC-08 | | |
| REMARK | 100 | | | |
| REMARK | 100 | THIS ENTRY HAS BEEN PROCESSED BY RCSB ON 27-OCT-10. | | |
| REMARK | 100 | THE RCSB ID CODE IS RCSB061626. | | |
| REMARK | 200 | | | |
| REMARK | 200 | EXPERIMENTAL DETAILS | | |
| REMARK | 200 | EXPERIMENT TYPE | | : X-RAY DIFFRACTION |
| REMARK | 200 | DATE OF DATA COLLECTION | | : 16-NOV-08; 13-NOV-09 |
| REMARK | 200 | TEMPERATURE | (KELVIN) | : 100; 100 |
| REMARK | 200 | PH | | : NULL |
| REMARK | 200 | NUMBER OF CRYSTALS USED | | : 2 |
| REMARK | 200 | | | |
| REMARK | 200 | SYNCHROTRON | (Y/N) | : Y; Y |
| REMARK | 200 | RADIATION SOURCE | | : APS; APS |
| REMARK | 200 | BEAMLINE | | : 24-ID-E; 24-ID-E |
| REMARK | 200 | X-RAY GENERATOR MODEL | | : NULL; NULL |
| REMARK | 200 | MONOCHROMATIC OR LAUE | (M/L) | : M; M |
| REMARK | 200 | WAVELENGTH OR RANGE | (A) | : 0.9792; 0.9792 |
| REMARK | 200 | MONOCHROMATOR | | : NULL; NULL |
| REMARK | 200 | OPTICS | | : NULL; NULL |
| REMARK | 200 | | | |
| REMARK | 200 | DETECTOR TYPE | | : CCD; CCD |
| REMARK | 200 | DETECTOR MANUFACTURER | | : ADSC QUANTUM 315; ADSC QUANTUM |
| REMARK | 200 | 315 | | |
| REMARK | 200 | INTENSITY-INTEGRATION SOFTWARE | | : DENZO |
| REMARK | 200 | DATA SCALING SOFTWARE | | : SCALEPACK |
| REMARK | 200 | | | |
| REMARK | 200 | NUMBER OF UNIQUE REFLECTIONS | | : 1870 |
| REMARK | 200 | RESOLUTION RANGE HIGH | (A) | : 1.800 |
| REMARK | 200 | RESOLUTION RANGE LOW | (A) | : 90.000 |
| REMARK | 200 | REJECTION CRITERIA | (SIGMA(I)) | : −3.000 |
| REMARK | 200 | | | |
| REMARK | 200 | OVERALL. | | |
| REMARK | 200 | COMPLETENESS FOR RANGE | (%) | : 91.5 |
| REMARK | 200 | DATA REDUNDANCY | | : 3.800 |
| REMARK | 200 | R MERGE | (I) | : 0.18900 |
| REMARK | 200 | R SYM | (I) | : NULL |

| | | | |
|---|---|---|---|
| REMARK | 200 | <I/SIGMA(I)> FOR THE DATA SET | : 8.2000 |
| REMARK | 200 | | |
| REMARK | 200 | IN THE HIGHEST RESOLUTION SHELL. | |

| | | | | |
|---|---|---|---|---|
| REMARK | 200 | HIGHEST RESOLUTION SHELL, RANGE HIGH | (A) | : 1.80 |
| REMARK | 200 | HIGHEST RESOLUTION SHELL, RANGE LOW | (A) | : 1.94 |

| | | | | |
|---|---|---|---|---|
| REMARK | 200 | COMPLETENESS FOR SHELL | (%) | : 71.7 |
| REMARK | 200 | DATA REDUNDANCY IN SHELL | | : 1.70 |
| REMARK | 200 | R MERGE FOR SHELL | (I) | : 0.41400 |
| REMARK | 200 | R SYM FOR SHELL | (I) | : NULL |
| REMARK | 200 | <I/SIGMA(I)> FOR SHELL | | : NULL |
| REMARK | 200 | | | |

| REMARK | 200 | DIFFRACTION PROTOCOL: SINGLE WAVELENGTH; SINGLE WAVELENGTH | | |
|---|---|---|---|---|
| REMARK | 200 | METHOD USED TO DETERMINE THE STRUCTURE: MOLECULAR REPLACEMENT | | |
| REMARK | 200 | SOFTWARE USED: PHASER | | |
| REMARK | 200 | STARTING MODEL: NULL | | |
| REMARK | 200 | | | |
| REMARK | 200 | REMARK: NULL | | |
| REMARK | 280 | | | |
| REMARK | 280 | CRYSTAL | | |
| REMARK | 280 | SOLVENT CONTENT, VS (%): 37.01 | | |
| REMARK | 280 | MATTHEWS COEFFICIENT, VM (ANGSTROMS**3/DA): 1.95 | | |
| REMARK | 280 | | | |
| REMARK | 280 | CRYSTALLIZATION CONDITIONS: RESERVOIR CONTAINED 30% W/V | | |
| REMARK | 280 | POLYETHYLENE GLYCOL 1,500, 20% V/V GLYCEROL, VAPOR DIFFUSION, | | |
| REMARK | 280 | HANGING DROP, TEMPERATURE 291K. RESERVOIR CONTAINED 10% W/V | | |
| REMARK | 280 | POLYETHYLENE GLYCOL 1,500, 30% V/V GLYCEROL, VAPOR DIFFUSION, | | |
| REMARK | 280 | HANGING DROP, TEMPERATURE 291K | | |
| REMARK | 290 | | | |
| REMARK | 290 | CRYSTALLOGRAPHIC SYMMETRY | | |
| REMARK | 290 | SYMMETRY OPERATORS FOR SPACE GROUP: P 1 | | |
| REMARK | 290 | | | |
| REMARK | 290 | | SYMOP | SYMMETRY |
| REMARK | 290 | | NNNMMM | OPERATOR |
| REMARK | 290 | | 1555 | X, Y, Z |
| REMARK | 290 | | | |

| | | | |
|---|---|---|---|
| REMARK | 290 | WHERE | NNN -> OPERATOR NUMBER |
| REMARK | 290 | | MMM -> TRANSLATION VECTOR |
| REMARK | 290 | | |

| REMARK | 290 | CRYSTALLOGRAPHIC SYMMETRY TRANSFORMATIONS | | | | | |
|---|---|---|---|---|---|---|---|
| REMARK | 290 | THE FOLLOWING TRANSFORMATIONS OPERATE ON THE ATOM/HETATM | | | | | |
| REMARK | 290 | RECORDS IN THIS ENTRY TO PRODUCE CRYSTALLOGRAPHICALLY | | | | | |
| REMARK | 290 | RELATED MOLECULES. | | | | | |
| REMARK | 290 | SMTRY1 | 1 | 1.000000 | 0.000000 | 0.000000 | 0.00000 |
| REMARK | 290 | SMTRY2 | 1 | 0.000000 | 1.000000 | 0.000000 | 0.00000 |
| REMARK | 290 | SMTRY3 | 1 | 0.000000 | 0.000000 | 1.000000 | 0.00000 |
| REMARK | 290 | | | | | | |
| REMARK | 290 | REMARK: NULL | | | | | |
| REMARK | 300 | | | | | | |
| REMARK | 300 | BIOMOLECULE: 1 | | | | | |
| REMARK | 300 | SEE REMARK 350 FOR THE AUTHOR PROVIDED AND/OR PROGRAM | | | | | |
| REMARK | 300 | GENERATED ASSEMBLY INFORMATION FOR THE STRUCTURE IN | | | | | |
| REMARK | 300 | THIS ENTRY. THE REMARK MAY ALSO PROVIDE INFORMATION ON | | | | | |
| REMARK | 300 | BURIED SURFACE AREA. | | | | | |
| REMARK | 300 | REMARK: THE BIOLOGICAL UNIT IS A PAIR OF BETA SHEETS WITH ORANGE G | | | | | |
| REMARK | 300 | INTERRELATING BETWEEN THE SHEETS. THE FIBER IS CONSTRUCTED FROM | | | | | |
| REMARK | 300 | UNIT CELL TRANSLATIONS ALONG THE A DIRECTION (I.E. X + 1, Y, Z; X + 2, Y, | | | | | |
| REMARK | 300 | Z; X + 3, Y, Z, ETC.). | | | | | |
| REMARK | 350 | | | | | | |
| REMARK | 350 | COORDINATES FOR A COMPLETE MULTIMER REPRESENTING THE KNOWN | | | | | |
| REMARK | 350 | BIOLOGICALLY SIGNIFICANT OLIGOMERIZATION STATE OF THE | | | | | |
| REMARK | 350 | MOLECULE CAN BE GENERATED BY APPLYING BIOMT TRANSFORMATIONS | | | | | |
| REMARK | 350 | GIVEN BELOW. BOTH NON-CRYSTALLOGRAPHIC AND | | | | | |
| REMARK | 350 | CRYSTALLOGRAPHIC OPERATIONS ARE GIVEN. | | | | | |
| REMARK | 350 | | | | | | |
| REMARK | 350 | BIOMOLECULE: 1 | | | | | |
| REMARK | 350 | AUTHOR DETERMINED BIOLOGICAL UNIT: TETRAMERIC | | | | | |
| REMARK | 350 | APPLY THE FOLLOWING TO CHAINS: A, B, C, D | | | | | |
| REMARK | 350 | BIOMT1 | 1 | 1.000000 | 0.000000 | 0.000000 | 0.00000 |
| REMARK | 350 | BIOMT2 | 1 | 0.000000 | 1.000000 | 0.000000 | 0.00000 |
| REMARK | 350 | BIOMT3 | 1 | 0.000000 | 0.000000 | 1.000000 | 0.00000 |
| REMARK | 800 | | | | | | |

| | | |
|---|---|---|
| REMARK | 800 | SITE |
| REMARK | 800 | SITE_IDENTIFIER: AC1 |
| REMARK | 800 | EVIDENCE_CODE: SOFTWARE |
| REMARK | 800 | SITE_DESCRIPTION: BINDING SITE FOR RESIDUE ORA B 49 |
| REMARK | 800 | |
| REMARK | 800 | SITE_IDENTIFIER: AC2 |
| REMARK | 800 | EVIDENCE_CODE: SOFTWARE |
| REMARK | 800 | SITE_DESCRIPTION: BINDING SITE FOR RESIDUE ORA D 50 |
| REMARK | 900 | |
| REMARK | 900 | RELATED ENTRIES |
| REMARK | 900 | RELATED ID: 3OW9 RELATED DB: PDB |
| REMARK | 900 | STRUCTURE OF AN AMYLOID FORMING PEPTIDE KLVFFA FROM AMYLOID |
| REMARK | 900 | BETA, ALTERNATE POLYMORPH II |

| | | | | | | | | |
|---|---|---|---|---|---|---|---|---|
| DBREF | 3OVJ A | 1 | 6 | UNP | P05067 | A4_HUMAN | 687 | 692 |
| DBREF | 3OVJ B | 1 | 6 | UNP | P05067 | A4_HUMAN | 687 | 692 |
| DBREF | 3OVJ C | 1 | 6 | UNP | P05067 | A4_HUMAN | 687 | 692 |
| DBREF | 3OVJ D | 1 | 6 | UNP | P05067 | A4_HUMAN | 687 | 692 |

| | | | | | | | | | |
|---|---|---|---|---|---|---|---|---|---|
| SEQRES | 1 | A | 6 | LYS | LEU | VAL | PHE | PHE | ALA |
| SEQRES | 1 | B | 6 | LYS | LEU | VAL | PHE | PHE | ALA |
| SEQRES | 1 | C | 6 | LYS | LEU | VAL | PHE | PHE | ALA |
| SEQRES | 1 | D | 6 | LYS | LEU | VAL | PHE | PHE | ALA |

| | | | |
|---|---|---|---|
| HET | ORA B | 49 | 27 |
| HET | ORA D | 50 | 27 |

| HETNAM | | ORA 7-HYDROXY-8-[(E)-PHENYLDIAZENYL]NAPHTHALENE-1,3- | | | | | | | | | | | | | | | | |
|---|---|---|---|---|---|---|---|---|---|---|---|---|---|---|---|---|---|---|
| HETNAM | 2 | ORA DISULFONIC ACID | | | | | | | | | | | | | | | | |
| HETSYN | | ORA ORANGE G | | | | | | | | | | | | | | | | |
| FORMUL | 5 | ORA 2(C16 H12 N2 O7 S2) | | | | | | | | | | | | | | | | |
| FORMUL | 7 | HOH *11(H2 O) | | | | | | | | | | | | | | | | |
| SHEET | 1 | A | 2 | LEU | A | 2 | PHE | A | 5 | 0 | | | | | | | | |
| SHEET | 2 | A | 2 | LEU | B | 2 | PHE | B | 5 | −1 | O | PHE | B | 5 | N | LEU | A | 2 |
| SHEET | 1 | B | 2 | LEU | C | 2 | PHE | C | 5 | 0 | | | | | | | | |
| SHEET | 2 | B | 2 | LEU | D | 2 | PHE | D | 5 | −1 | O | PHE | D | 5 | N | LEU | C | 2 |

| | | | | | | | | | | | | | | | |
|---|---|---|---|---|---|---|---|---|---|---|---|---|---|---|---|
| SITE | 1 | AC1 | 9 | LYS | A | 1 | PHE | A | 4 | LYS | B | 1 | VAL | B | 3 |
| SITE | 2 | AC1 | 9 | LYS | C | 1 | VAL | C | 3 | HOH | C | 10 | LYS | D | 1 |
| SITE | 3 | AC1 | 9 | PHE | D | 4 | | | | | | | | | |
| SITE | 1 | AC2 | 8 | LYS | A | 1 | VAL | A | 3 | PHE | B | 4 | LYS | C | 1 |
| SITE | 2 | AC2 | 8 | PHE | C | 4 | HOH | C | 10 | LYS | D | 1 | VAL | D | 3 |

| | | | |
|---|---|---|---|
| CRYST1 | 9.536 26.008 25.803 62.28 88.59 88.45 | P 1 | 4 |

| | | | | |
|---|---|---|---|---|
| ORIGX1 | 1.000000 | 0.000000 | 0.000000 | 0.00000 |
| ORIGX2 | 0.000000 | 1.000000 | 0.000000 | 0.00000 |
| ORIGX3 | 0.000000 | 0.000000 | 1.000000 | 0.00000 |
| SCALE1 | 0.104866 | −0.002843 | −0.001414 | 0.00000 |
| SCALE2 | 0.000000 | 0.038464 | −0.020193 | 0.00000 |
| SCALES | 0.000000 | 0.000000 | 0.043785 | 0.00000 |

| | | | | | | | | | | | |
|---|---|---|---|---|---|---|---|---|---|---|---|
| ATOM | 1 | N | LYS | A | 1 | 2.324 | −14.883 | −14.699 | 1.00 | 15.17 | N |
| ATOM | 2 | CA | LYS | A | 1 | 1.869 | −14.674 | −13.291 | 1.00 | 14.33 | C |
| ATOM | 3 | C | LYS | A | 1 | 2.420 | −13.336 | −12.782 | 1.00 | 12.20 | C |
| ATOM | 4 | O | LYS | A | 1 | 3.625 | −13.187 | −12.656 | 1.00 | 12.01 | O |
| ATOM | 5 | CB | LYS | A | 1 | 2.373 | −15.837 | −12.428 | 1.00 | 14.63 | C |
| ATOM | 6 | CG | LYS | A | 1 | 1.747 | −15.963 | −11.053 | 1.00 | 18.91 | C |
| ATOM | 7 | CD | LYS | A | 1 | 2.266 | −17.236 | −10.388 | 1.00 | 21.97 | C |
| ATOM | 8 | CE | LYS | A | 1 | 1.388 | −17.689 | −9.258 | 1.00 | 25.93 | C |
| ATOM | 9 | NZ | LYS | A | 1 | 1.592 | −19.126 | −8.914 | 1.00 | 26.35 | N |
| ATOM | 10 | N | LEU | A | 2 | 1.540 | −12.373 | −12.514 | 1.00 | 11.30 | N |
| ATOM | 11 | CA | LEU | A | 2 | 1.919 | −11.069 | −11.918 | 1.00 | 10.77 | C |
| ATOM | 12 | C | LEU | A | 2 | 1.354 | −10.927 | −10.501 | 1.00 | 8.72 | C |
| ATOM | 13 | O | LEU | A | 2 | 0.156 | −11.092 | −10.294 | 1.00 | 9.35 | O |
| ATOM | 14 | CB | LEU | A | 2 | 1.389 | −9.880 | −12.746 | 1.00 | 9.87 | C |
| ATOM | 15 | CG | LEU | A | 2 | 1.608 | −8.483 | −12.114 | 1.00 | 13.36 | C |
| ATOM | 16 | CD1 | LEU | A | 2 | 3.091 | −8.081 | −12.262 | 1.00 | 13.10 | C |
| ATOM | 17 | CD2 | LEU | A | 2 | 0.668 | −7.396 | −12.680 | 1.00 | 15.58 | C |
| ATOM | 18 | N | VAL | A | 3 | 2.213 | −10.559 | −9.560 | 1.00 | 7.72 | N |
| ATOM | 19 | CA | VAL | A | 3 | 1.794 | −10.172 | −8.218 | 1.00 | 7.67 | C |
| ATOM | 20 | C | VAL | A | 3 | 2.433 | −8.796 | −7.909 | 1.00 | 8.47 | C |
| ATOM | 21 | O | VAL | A | 3 | 3.652 | −8.608 | −8.060 | 1.00 | 7.48 | O |
| ATOM | 22 | CB | VAL | A | 3 | 2.188 | −11.246 | −7.163 | 1.00 | 7.02 | C |
| ATOM | 23 | CG1 | VAL | A | 3 | 1.685 | −10.857 | −5.766 | 1.00 | 9.90 | C |
| ATOM | 24 | CG2 | VAL | A | 3 | 1.649 | −12.615 | −7.542 | 1.00 | 4.63 | C |
| ATOM | 25 | N | PHE | A | 4 | 1.591 | −7.838 | −7.517 | 1.00 | 10.07 | N |
| ATOM | 26 | CA | PHE | A | 4 | 1.993 | −6.423 | −7.286 | 1.00 | 11.69 | C |
| ATOM | 27 | C | PHE | A | 4 | 1.338 | −5.865 | −6.013 | 1.00 | 11.61 | C |
| ATOM | 28 | O | PHE | A | 4 | 0.114 | −5.721 | −5.953 | 1.00 | 9.57 | O |
| ATOM | 29 | CB | PHE | A | 4 | 1.617 | −5.558 | −8.514 | 1.00 | 11.35 | C |
| ATOM | 30 | CG | PHE | A | 4 | 1.758 | −4.060 | −8.303 | 1.00 | 14.32 | C |
| ATOM | 31 | CD1 | PHE | A | 4 | 2.920 | −3.522 | −7.764 | 1.00 | 20.30 | C |
| ATOM | 32 | CD2 | PHE | A | 4 | 0.738 | −3.191 | −8.693 | 1.00 | 19.06 | C |
| ATOM | 33 | CE1 | PHE | A | 4 | 3.058 | −2.144 | −7.575 | 1.00 | 21.64 | C |
| ATOM | 34 | CE2 | PHE | A | 4 | 0.869 | −1.812 | −8.522 | 1.00 | 17.52 | C |
| ATOM | 35 | CZ | PHE | A | 4 | 2.034 | −1.288 | −7.959 | 1.00 | 19.78 | C |
| ATOM | 36 | N | PHE | A | 5 | 2.164 | −5.584 | −5.004 | 1.00 | 13.15 | N |
| ATOM | 37 | CA | PHE | A | 5 | 1.731 | −4.929 | −3.768 | 1.00 | 14.89 | C |
| ATOM | 38 | C | PHE | A | 5 | 2.295 | −3.503 | −3.769 | 1.00 | 15.69 | C |
| ATOM | 39 | O | PHE | A | 5 | 3.506 | −3.324 | −3.909 | 1.00 | 14.09 | O |
| ATOM | 40 | CB | PHE | A | 5 | 2.284 | −5.655 | −2.538 | 1.00 | 15.28 | C |
| ATOM | 41 | CG | PHE | A | 5 | 1.632 | −6.984 | −2.228 | 1.00 | 18.94 | C |
| ATOM | 42 | CD1 | PHE | A | 5 | 1.154 | −7.827 | −3.226 | 1.00 | 25.00 | C |
| ATOM | 43 | CD2 | PHE | A | 5 | 1.550 | −7.414 | −0.913 | 1.00 | 26.74 | C |
| ATOM | 44 | CE1 | PHE | A | 5 | 0.575 | −9.064 | −2.907 | 1.00 | 29.55 | C |
| ATOM | 45 | CE2 | PHE | A | 5 | 0.969 | −8.655 | −0.590 | 1.00 | 31.00 | C |
| ATOM | 46 | CZ | PHE | A | 5 | 0.485 | −9.472 | −1.587 | 1.00 | 29.17 | C |
| ATOM | 47 | N | ALA | A | 6 | 1.437 | −2.498 | −3.573 | 1.00 | 17.34 | N |
| ATOM | 48 | CA | ALA | A | 6 | 1.870 | −1.083 | −3.552 | 1.00 | 18.89 | C |
| ATOM | 49 | C | ALA | A | 6 | 1.320 | −0.339 | −2.342 | 1.00 | 20.80 | C |
| ATOM | 50 | O | ALA | A | 6 | 0.296 | −0.739 | −1.771 | 1.00 | 22.76 | O |
| ATOM | 51 | CB | ALA | A | 6 | 1.441 | −0.381 | −4.824 | 1.00 | 18.51 | C |
| ATOM | 52 | OXT | ALA | A | 6 | 1.890 | 0.678 | −1.911 | 1.00 | 21.85 | O |
| TER | 53 | | ALA | A | 6 | | | | | | |
| ATOM | 54 | N | LYS | B | 1 | −2.658 | −1.611 | −1.505 | 1.00 | 11.14 | N |
| ATOM | 55 | CA | LYS | B | 1 | −3.063 | −1.977 | −2.892 | 1.00 | 10.97 | C |
| ATOM | 56 | C | LYS | B | 1 | −2.436 | −3.302 | −3.290 | 1.00 | 11.06 | C |
| ATOM | 57 | O | LYS | B | 1 | −1.218 | −3.478 | −3.164 | 1.00 | 11.09 | O |
| ATOM | 58 | CB | LYS | B | 1 | −2.631 | −0.885 | −3.853 | 1.00 | 11.34 | C |
| ATOM | 59 | CG | LYS | B | 1 | −3.085 | −1.079 | −5.259 | 1.00 | 13.85 | C |
| ATOM | 60 | CD | LYS | B | 1 | −2.793 | 0.167 | −6.064 | 1.00 | 21.29 | C |
| ATOM | 61 | CE | LYS | B | 1 | −3.894 | 0.485 | −7.026 | 1.00 | 22.74 | C |
| ATOM | 62 | NZ | LYS | B | 1 | −3.530 | 1.683 | −7.815 | 1.00 | 25.76 | N |
| ATOM | 63 | N | LEU | B | 2 | −3.267 | −4.225 | −3.781 | 1.00 | 10.74 | N |
| ATOM | 64 | CA | LEU | B | 2 | −2.818 | −5.541 | −4.258 | 1.00 | 9.68 | C |
| ATOM | 65 | C | LEU | B | 2 | −3.380 | −5.818 | −5.652 | 1.00 | 8.86 | C |
| ATOM | 66 | O | LEU | B | 2 | −4.574 | −5.651 | −5.874 | 1.00 | 8.12 | O |
| ATOM | 67 | CB | LEU | B | 2 | −3.284 | −6.629 | −3.279 | 1.00 | 9.16 | C |
| ATOM | 68 | CG | LEU | B | 2 | −3.026 | −8.119 | −3.535 | 1.00 | 11.76 | C |
| ATOM | 69 | CD1 | LEU | B | 2 | −3.106 | −8.901 | −2.226 | 1.00 | 17.59 | C |
| ATOM | 70 | CD2 | LEU | B | 2 | −4.027 | −8.699 | −4.520 | 1.00 | 15.49 | C |
| ATOM | 71 | N | VAL | B | 3 | −2.519 | −6.226 | −6.586 | 1.00 | 8.43 | N |
| ATOM | 72 | CA | VAL | B | 3 | −2.955 | −6.701 | −7.904 | 1.00 | 8.34 | C |
| ATOM | 73 | C | VAL | B | 3 | −2.358 | −8.090 | −8.169 | 1.00 | 7.81 | C |
| ATOM | 74 | O | VAL | B | 3 | −1.160 | −8.277 | −8.038 | 1.00 | 7.61 | O |
| ATOM | 75 | CB | VAL | B | 3 | −2.518 | −5.738 | −9.031 | 1.00 | 9.46 | C |
| ATOM | 76 | CG1 | VAL | B | 3 | −3.050 | −6.213 | −10.382 | 1.00 | 7.94 | C |
| ATOM | 77 | CG2 | VAL | B | 3 | −2.987 | −4.307 | −8.742 | 1.00 | 7.83 | C |
| ATOM | 78 | N | PHE | B | 4 | −3.216 | −9.061 | −8.496 | 1.00 | 8.14 | N |
| ATOM | 79 | CA | PHE | B | 4 | −2.799 | −10.406 | −8.933 | 1.00 | 8.94 | C |
| ATOM | 80 | C | PHE | B | 4 | −3.392 | −10.707 | −10.314 | 1.00 | 8.97 | C |
| ATOM | 81 | O | PHE | B | 4 | −4.568 | −10.426 | −10.570 | 1.00 | 7.58 | O |
| ATOM | 82 | CB | PHE | B | 4 | −3.229 | −11.492 | −7.924 | 1.00 | 8.51 | C |
| ATOM | 83 | CG | PHE | B | 4 | −3.071 | −12.919 | −8.437 | 1.00 | 9.16 | C |
| ATOM | 84 | CD1 | PHE | B | 4 | −1.871 | −13.608 | −8.313 | 1.00 | 8.74 | C |
| ATOM | 85 | CD2 | PHE | B | 4 | −4.132 | −13.567 | −9.040 | 1.00 | 9.69 | C |
| ATOM | 86 | CE1 | PHE | B | 4 | −1.744 | −14.924 | −8.783 | 1.00 | 8.88 | C |
| ATOM | 87 | CE2 | PHE | B | 4 | −4.010 | −14.877 | −9.515 | 1.00 | 9.12 | C |
| ATOM | 88 | CZ | PHE | B | 4 | −2.818 | −15.550 | −9.382 | 1.00 | 8.26 | C |
| ATOM | 89 | N | PHE | B | 5 | −2.577 | −11.290 | −11.185 | 1.00 | 9.27 | N |
| ATOM | 90 | CA | PHE | B | 5 | −3.057 | −11.767 | −12.477 | 1.00 | 10.91 | C |
| ATOM | 91 | C | PHE | B | 5 | −2.283 | −12.981 | −12.975 | 1.00 | 11.70 | C |
| ATOM | 92 | O | PHE | B | 5 | −1.052 | −12.951 | −13.046 | 1.00 | 11.51 | O |
| ATOM | 93 | CB | PHE | B | 5 | −3.006 | −10.687 | −13.558 | 1.00 | 10.96 | C |
| ATOM | 94 | CG | PHE | B | 5 | −3.759 | −11.080 | −14.790 | 1.00 | 12.64 | C |
| ATOM | 95 | CD1 | PHE | B | 5 | −5.131 | −10.883 | −14.843 | 1.00 | 15.91 | C |
| ATOM | 96 | CD2 | PHE | B | 5 | −3.130 | −11.732 | −15.839 | 1.00 | 16.42 | C |
| ATOM | 97 | CE1 | PHE | B | 5 | −5.862 | −11.277 | −15.944 | 1.00 | 17.62 | C |
| ATOM | 98 | CE2 | PHE | B | 5 | −3.852 | −12.136 | −16.942 | 1.00 | 19.57 | C |
| ATOM | 99 | CZ | PHE | B | 5 | −5.223 | −11.905 | −16.997 | 1.00 | 19.26 | C |
| ATOM | 100 | N | ALA | B | 6 | −3.020 | −14.040 | −13.326 | 1.00 | 13.65 | N |
| ATOM | 101 | CA | ALA | B | 6 | −2.436 | −15.273 | −13.868 | 1.00 | 15.22 | C |
| ATOM | 102 | C | ALA | B | 6 | −3.370 | −15.929 | −14.889 | 1.00 | 16.94 | C |
| ATOM | 103 | O | ALA | B | 6 | −4.591 | −15.815 | −14.733 | 1.00 | 19.20 | O |
| ATOM | 104 | CB | ALA | B | 6 | −2.141 | −16.234 | −12.753 | 1.00 | 15.26 | C |
| ATOM | 105 | OXT | ALA | B | 6 | −2.946 | −16.575 | −15.867 | 1.00 | 20.69 | O |
| TER | 106 | | ALA | B | 6 | | | | | | |
| ATOM | 107 | N | LYS | C | 1 | −4.519 | 7.726 | −16.689 | 1.00 | 14.05 | N |
| ATOM | 108 | CA | LYS | C | 1 | −3.958 | 6.421 | −16.240 | 1.00 | 13.16 | C |
| ATOM | 109 | C | LYS | C | 1 | −4.521 | 5.310 | −17.133 | 1.00 | 12.20 | C |
| ATOM | 110 | O | LYS | C | 1 | −5.724 | 5.117 | −17.172 | 1.00 | 12.20 | O |
| ATOM | 111 | CB | LYS | C | 1 | −4.333 | 6.185 | −14.776 | 1.00 | 14.70 | C |
| ATOM | 112 | CG | LYS | C | 1 | −3.533 | 5.109 | −14.064 | 1.00 | 17.57 | C |
| ATOM | 113 | CD | LYS | C | 1 | −4.232 | 4.681 | −12.780 | 1.00 | 20.38 | C |
| ATOM | 114 | CE | LYS | C | 1 | −3.250 | 4.519 | −11.644 | 1.00 | 24.06 | C |
| ATOM | 115 | NZ | LYS | C | 1 | −3.871 | 4.006 | −10.397 | 1.00 | 22.92 | N |
| ATOM | 116 | N | LEU | C | 2 | −3.646 | 4.605 | −17.852 | 1.00 | 11.29 | N |
| ATOM | 117 | CA | LEU | C | 2 | −4.033 | 3.455 | −18.715 | 1.00 | 11.13 | C |
| ATOM | 118 | C | LEU | C | 2 | −3.493 | 2.125 | −18.187 | 1.00 | 10.71 | C |
| ATOM | 119 | O | LEU | C | 2 | −2.288 | 1.998 | −17.918 | 1.00 | 11.60 | O |
| ATOM | 120 | CB | LEU | C | 2 | −3.504 | 3.628 | −20.156 | 1.00 | 10.99 | C |
| ATOM | 121 | CG | LEU | C | 2 | −3.755 | 2.488 | −21.154 | 1.00 | 10.45 | C |
| ATOM | 122 | CD1 | LEU | C | 2 | −5.243 | 2.402 | −21.515 | 1.00 | 10.00 | C |
| ATOM | 123 | CD2 | LEU | C | 2 | −2.902 | 2.660 | −22.409 | 1.00 | 10.02 | C |
| ATOM | 124 | N | VAL | C | 3 | −4.374 | 1.135 | −18.084 | 1.00 | 8.95 | N |
| ATOM | 125 | CA | VAL | C | 3 | −3.965 | −0.257 | −17.885 | 1.00 | 8.90 | C |
| ATOM | 126 | C | VAL | C | 3 | −4.610 | −1.121 | −18.967 | 1.00 | 9.38 | C |
| ATOM | 127 | O | VAL | C | 3 | −5.822 | −1.071 | −19.159 | 1.00 | 9.66 | O |
| ATOM | 128 | CB | VAL | C | 3 | −4.408 | −0.766 | −16.503 | 1.00 | 9.20 | C |
| ATOM | 129 | CG1 | VAL | C | 3 | −3.860 | −2.165 | −16.243 | 1.00 | 11.28 | C |
| ATOM | 130 | CG2 | VAL | C | 3 | −3.954 | 0.211 | −15.416 | 1.00 | 6.04 | C |
| ATOM | 131 | N | PHE | C | 4 | −3.786 | −1.900 | −19.663 | 1.00 | 11.18 | N |
| ATOM | 132 | CA | PHE | C | 4 | −4.189 | −2.746 | −20.811 | 1.00 | 12.40 | C |
| ATOM | 133 | C | PHE | C | 4 | −3.553 | −4.120 | −20.629 | 1.00 | 12.12 | C |
| ATOM | 134 | O | PHE | C | 4 | −2.326 | −4.215 | −20.561 | 1.00 | 10.79 | O |
| ATOM | 135 | CB | PHE | C | 4 | −3.722 | −2.108 | −22.147 | 1.00 | 11.55 | C |
| ATOM | 136 | CG | PHE | C | 4 | −3.848 | −3.009 | −23.362 | 1.00 | 11.86 | C |
| ATOM | 137 | CD1 | PHE | C | 4 | −4.982 | −3.802 | −23.548 | 1.00 | 18.98 | C |
| ATOM | 138 | CD2 | PHE | C | 4 | −2.862 | −3.025 | −24.344 | 1.00 | 14.46 | C |
| ATOM | 139 | CE1 | PHE | C | 4 | −5.118 | −4.625 | −24.674 | 1.00 | 17.54 | C |
| ATOM | 140 | CE2 | PHE | C | 4 | −2.986 | −3.842 | −25.475 | 1.00 | 17.83 | C |
| ATOM | 141 | CZ | PHE | C | 4 | −4.120 | −4.642 | −25.641 | 1.00 | 17.06 | C |
| ATOM | 142 | N | PHE | C | 5 | −4.384 | −5.165 | −20.525 | 1.00 | 12.42 | N |
| ATOM | 143 | CA | PHE | C | 5 | −3.923 | −6.565 | −20.480 | 1.00 | 14.30 | C |
| ATOM | 144 | C | PHE | C | 5 | −4.442 | −7.281 | −21.718 | 1.00 | 14.53 | C |
| ATOM | 145 | O | PHE | C | 5 | −5.650 | −7.306 | −21.936 | 1.00 | 12.00 | O |
| ATOM | 146 | CB | PHE | C | 5 | −4.492 | −7.301 | −19.260 | 1.00 | 14.35 | C |
| ATOM | 147 | CG | PHE | C | 5 | −3.850 | −6.952 | −17.953 | 1.00 | 16.94 | C |
| ATOM | 148 | CD1 | PHE | C | 5 | −3.286 | −5.712 | −17.710 | 1.00 | 20.76 | C |
| ATOM | 149 | CD2 | PHE | C | 5 | −3.842 | −7.880 | −16.937 | 1.00 | 25.20 | C |
| ATOM | 150 | CE1 | PHE | C | 5 | −2.705 | −5.426 | −16.481 | 1.00 | 25.34 | C |
| ATOM | 151 | CE2 | PHE | C | 5 | −3.260 | −7.591 | −15.701 | 1.00 | 28.16 | C |
| ATOM | 152 | CZ | PHE | C | 5 | −2.696 | −6.370 | −15.479 | 1.00 | 26.01 | C |
| ATOM | 153 | N | ALA | C | 6 | −3.550 | −7.875 | −22.509 | 1.00 | 15.55 | N |
| ATOM | 154 | CA | ALA | C | 6 | −3.945 | −8.580 | −23.744 | 1.00 | 17.57 | C |
| ATOM | 155 | C | ALA | C | 6 | −3.387 | −10.006 | −23.818 | 1.00 | 19.47 | C |
| ATOM | 156 | O | ALA | C | 6 | −2.297 | −10.310 | −23.303 | 1.00 | 21.29 | O |
| ATOM | 157 | CB | ALA | C | 6 | −3.491 | −7.790 | −24.965 | 1.00 | 17.93 | C |
| ATOM | 158 | OXT | ALA | C | 6 | −4.030 | −10.891 | −24.401 | 1.00 | 20.57 | O |
| TER | 159 | | ALA | C | 6 | | | | | | |
| ATOM | 160 | N | LYS | D | 1 | 0.313 | −10.058 | −22.448 | 1.00 | 15.69 | N |
| ATOM | 161 | CA | LYS | D | 1 | 0.849 | −8.684 | −22.688 | 1.00 | 14.68 | C |
| ATOM | 162 | C | LYS | D | 1 | 0.285 | −7.762 | −21.654 | 1.00 | 13.06 | C |
| ATOM | 163 | O | LYS | D | 1 | −0.911 | −7.802 | −21.398 | 1.00 | 11.09 | O |
| ATOM | 164 | CB | LYS | D | 1 | 0.426 | −8.181 | −24.066 | 1.00 | 15.52 | C |
| ATOM | 165 | CG | LYS | D | 1 | 0.927 | −6.806 | −24.445 | 1.00 | 19.82 | C |
| ATOM | 166 | CD | LYS | D | 1 | 0.656 | −6.552 | −25.912 | 1.00 | 23.85 | C |
| ATOM | 167 | CE | LYS | D | 1 | 1.923 | −6.294 | −26.697 | 1.00 | 26.53 | C |
| ATOM | 168 | NZ | LYS | D | 1 | 1.668 | −6.369 | −28.172 | 1.00 | 29.12 | N |
| ATOM | 169 | N | LEU | D | 2 | 1.137 | −6.915 | −21.079 | 1.00 | 12.33 | N |
| ATOM | 170 | CA | LEU | D | 2 | 0.703 | −5.890 | −20.115 | 1.00 | 11.86 | C |
| ATOM | 171 | C | LEU | D | 2 | 1.248 | −4.515 | −20.498 | 1.00 | 10.38 | C |
| ATOM | 172 | O | LEU | D | 2 | 2.442 | −4.387 | −20.744 | 1.00 | 10.47 | O |
| ATOM | 173 | CB | LEU | D | 2 | 1.180 | −6.267 | −18.713 | 1.00 | 12.34 | C |
| ATOM | 174 | CG | LEU | D | 2 | 0.827 | −5.350 | −17.551 | 1.00 | 14.82 | C |
| ATOM | 175 | CD1 | LEU | D | 2 | 0.871 | −6.145 | −16.234 | 1.00 | 17.79 | C |
| ATOM | 176 | CD2 | LEU | D | 2 | 1.802 | −4.204 | −17.474 | 1.00 | 18.28 | C |
| ATOM | 177 | N | VAL | D | 3 | 0.376 | −3.498 | −20.544 | 1.00 | 9.81 | N |
| ATOM | 178 | CA | VAL | D | 3 | 0.785 | −2.106 | −20.789 | 1.00 | 8.31 | C |
| ATOM | 179 | C | VAL | D | 3 | 0.180 | −1.205 | −19.720 | 1.00 | 8.98 | C |
| ATOM | 180 | O | VAL | D | 3 | −1.013 | −1.256 | −19.471 | 1.00 | 7.44 | O |
| ATOM | 181 | CB | VAL | D | 3 | 0.370 | −1.634 | −22.202 | 1.00 | 9.98 | C |
| ATOM | 182 | CG1 | VAL | D | 3 | 0.823 | −0.199 | −22.463 | 1.00 | 8.03 | C |
| ATOM | 183 | CG2 | VAL | D | 3 | 0.920 | −2.598 | −23.291 | 1.00 | 6.92 | C |
| ATOM | 184 | N | PHE | D | 4 | 1.042 | −0.436 | −19.042 | 1.00 | 9.25 | N |
| ATOM | 185 | CA | PHE | D | 4 | 0.642 | 0.555 | −18.028 | 1.00 | 9.33 | C |
| ATOM | 186 | C | PHE | D | 4 | 1.269 | 1.912 | −18.354 | 1.00 | 8.88 | C |
| ATOM | 187 | O | PHE | D | 4 | 2.458 | 2.007 | −18.666 | 1.00 | 7.98 | O |
| ATOM | 188 | CB | PHE | D | 4 | 1.044 | 0.129 | −16.598 | 1.00 | 8.45 | C |
| ATOM | 189 | CG | PHE | D | 4 | 0.949 | 1.242 | −15.577 | 1.00 | 8.55 | C |
| ATOM | 190 | CD1 | PHE | D | 4 | −0.245 | 1.516 | −14.924 | 1.00 | 8.62 | C |
| ATOM | 191 | CD2 | PHE | D | 4 | 2.052 | 2.020 | −15.280 | 1.00 | 8.13 | C |
| ATOM | 192 | CE1 | PHE | D | 4 | −0.315 | 2.555 | −13.978 | 1.00 | 9.38 | C |
| ATOM | 193 | CE2 | PHE | D | 4 | 1.982 | 3.050 | −14.358 | 1.00 | 11.01 | C |
| ATOM | 194 | CZ | PHE | D | 4 | 0.807 | 3.317 | −13.707 | 1.00 | 9.91 | C |
| ATOM | 195 | N | PHE | D | 5 | 0.454 | 2.959 | −18.292 | 1.00 | 9.26 | N |
| ATOM | 196 | CA | PHE | D | 5 | 0.948 | 4.327 | −18.444 | 1.00 | 11.02 | C |
| ATOM | 197 | C | PHE | D | 5 | 0.190 | 5.319 | −17.568 | 1.00 | 11.59 | C |
| ATOM | 198 | O | PHE | D | 5 | −1.038 | 5.378 | −17.598 | 1.00 | 11.90 | O |
| ATOM | 199 | CB | PHE | D | 5 | 0.906 | 4.827 | −19.896 | 1.00 | 10.79 | C |
| ATOM | 200 | CG | PHE | D | 5 | 1.479 | 6.209 | −20.034 | 1.00 | 14.40 | C |
| ATOM | 201 | CD1 | PHE | D | 5 | 2.841 | 6.380 | −20.201 | 1.00 | 18.36 | C |
| ATOM | 202 | CD2 | PHE | D | 5 | 0.679 | 7.328 | −19.873 | 1.00 | 17.09 | C |
| ATOM | 203 | CE1 | PHE | D | 5 | 3.397 | 7.647 | −20.260 | 1.00 | 21.89 | C |
| ATOM | 204 | CE2 | PHE | D | 5 | 1.223 | 8.598 | −19.936 | 1.00 | 20.56 | C |
| ATOM | 205 | CZ | PHE | D | 5 | 2.578 | 8.758 | −20.134 | 1.00 | 21.09 | C |
| ATOM | 206 | N | ALA | D | 6 | 0.941 | 6.109 | −16.803 | 1.00 | 13.80 | N |
| ATOM | 207 | CA | ALA | D | 6 | 0.370 | 7.137 | −15.940 | 1.00 | 16.42 | C |
| ATOM | 208 | C | ALA | D | 6 | 1.247 | 8.401 | −15.918 | 1.00 | 19.12 | C |
| ATOM | 209 | O | ALA | D | 6 | 2.477 | 8.315 | −16.022 | 1.00 | 20.34 | O |
| ATOM | 210 | CB | ALA | D | 6 | 0.208 | 6.596 | −14.545 | 1.00 | 15.64 | C |
| ATOM | 211 | OXT | ALA | D | 6 | 0.757 | 9.536 | −15.785 | 1.00 | 21.13 | O |
| TER | 212 | | ALA | D | 6 | | | | | | |
| HETATM | 213 | C1 | ORA | B | 49 | −6.133 | −3.861 | −11.927 | 1.00 | 16.36 | C |
| HETATM | 214 | N1 | ORA | B | 49 | −2.242 | −2.368 | −12.209 | 1.00 | 16.83 | N |
| HETATM | 215 | O1 | ORA | B | 49 | 3.227 | 4.028 | −10.418 | 1.00 | 39.53 | O |
| HETATM | 216 | S1 | ORA | B | 49 | 2.588 | 3.141 | −9.423 | 1.00 | 35.98 | S |
| HETATM | 217 | C2 | ORA | B | 49 | −5.702 | −2.859 | −11.057 | 1.00 | 16.06 | C |
| HETATM | 218 | N2 | ORA | B | 49 | −1.893 | −1.190 | −12.326 | 1.00 | 21.60 | N |
| HETATM | 219 | O2 | ORA | B | 49 | −2.734 | 1.198 | −11.709 | 1.00 | 21.50 | O |
| HETATM | 220 | S2 | ORA | B | 49 | −1.965 | 1.145 | −10.455 | 1.00 | 25.05 | S |
| HETATM | 221 | C3 | ORA | B | 49 | −5.278 | −4.357 | −12.895 | 1.00 | 14.58 | C |
| HETATM | 222 | O3 | ORA | B | 49 | 1.701 | 3.945 | −8.556 | 1.00 | 39.52 | O |
| HETATM | 223 | C4 | ORA | B | 49 | 2.373 | −0.664 | −12.578 | 1.00 | 17.80 | C |
| HETATM | 224 | O4 | ORA | B | 49 | 3.597 | 2.499 | −8.566 | 1.00 | 34.07 | O |
| HETATM | 225 | C5 | ORA | B | 49 | −4.416 | −2.352 | −11.145 | 1.00 | 12.20 | C |
| HETATM | 226 | O5 | ORA | B | 49 | −2.477 | 0.030 | −9.650 | 1.00 | 26.49 | O |
| HETATM | 227 | C6 | ORA | B | 49 | −3.982 | −3.853 | −12.982 | 1.00 | 17.98 | C |
| HETATM | 228 | O6 | ORA | B | 49 | −2.198 | 2.407 | −9.735 | 1.00 | 25.85 | O |
| HETATM | 229 | C7 | ORA | B | 49 | 1.743 | −1.653 | −13.303 | 1.00 | 18.63 | C |
| HETATM | 230 | O7 | ORA | B | 49 | −0.206 | −2.807 | −13.962 | 1.00 | 25.99 | O |
| HETATM | 231 | C8 | ORA | B | 49 | 2.394 | 1.143 | −11.061 | 1.00 | 24.85 | C |
| HETATM | 232 | C9 | ORA | B | 49 | 0.384 | 1.969 | −10.076 | 1.00 | 26.37 | C |
| HETATM | 233 | C10 | ORA | B | 49 | 1.653 | 0.182 | −11.747 | 1.00 | 20.98 | C |
| HETATM | 234 | C11 | ORA | B | 49 | 0.255 | 0.092 | −11.610 | 1.00 | 19.95 | C |
| HETATM | 235 | C12 | ORA | B | 49 | −3.557 | −2.838 | −12.121 | 1.00 | 16.56 | C |
| HETATM | 236 | C13 | ORA | B | 49 | −0.466 | −0.953 | −12.365 | 1.00 | 17.22 | C |
| HETATM | 237 | C14 | ORA | B | 49 | 0.377 | −1.821 | −13.221 | 1.00 | 21.24 | C |
| HETATM | 238 | C15 | ORA | B | 49 | 1.758 | 2.035 | −10.219 | 1.00 | 30.05 | C |
| HETATM | 239 | C16 | ORA | B | 49 | −0.384 | 1.018 | −10.745 | 1.00 | 24.11 | C |
| HETATM | 240 | C1 | ORA | D | 50 | 4.178 | 0.067 | −25.120 | 1.00 | 19.60 | C |
| HETATM | 241 | N1 | ORA | D | 50 | 0.266 | −0.221 | −26.521 | 1.00 | 17.92 | N |
| HETATM | 242 | O1 | ORA | D | 50 | −4.247 | −5.590 | −31.253 | 1.00 | 37.19 | O |
| HETATM | 243 | S1 | ORA | D | 50 | −4.723 | −5.151 | −29.927 | 1.00 | 34.45 | S |
| HETATM | 244 | C2 | ORA | D | 50 | 3.703 | −1.139 | −25.631 | 1.00 | 16.22 | C |
| HETATM | 245 | N2 | ORA | D | 50 | −0.066 | −0.583 | −27.660 | 1.00 | 20.81 | N |
| HETATM | 246 | O2 | ORA | D | 50 | 0.783 | −2.376 | −29.297 | 1.00 | 21.17 | O |
| HETATM | 247 | S2 | ORA | D | 50 | −0.086 | −3.449 | −28.785 | 1.00 | 23.07 | S |
| HETATM | 248 | C3 | ORA | D | 50 | 3.354 | 1.183 | −25.082 | 1.00 | 15.75 | C |
| HETATM | 249 | O3 | ORA | D | 50 | −4.629 | −6.286 | −28.989 | 1.00 | 37.22 | O |
| HETATM | 250 | C4 | ORA | D | 50 | −4.319 | −0.507 | −28.200 | 1.00 | 16.91 | C |
| HETATM | 251 | O4 | ORA | D | 50 | −6.147 | −4.746 | −30.005 | 1.00 | 39.44 | O |
| HETATM | 252 | C5 | ORA | D | 50 | 2.405 | −1.236 | −26.097 | 1.00 | 16.30 | C |
| HETATM | 253 | O5 | ORA | D | 50 | 0.361 | −3.736 | −27.417 | 1.00 | 24.59 | O |
| HETATM | 254 | C6 | ORA | D | 50 | 2.054 | 1.085 | −25.554 | 1.00 | 18.40 | C |
| HETATM | 255 | O6 | ORA | D | 50 | 0.105 | −4.602 | −29.676 | 1.00 | 22.54 | O |
| HETATM | 256 | C7 | ORA | D | 50 | −3.655 | 0.595 | −27.706 | 1.00 | 20.93 | C |
| HETATM | 257 | O7 | ORA | D | 50 | −1.654 | 1.666 | −27.060 | 1.00 | 29.85 | O |
| HETATM | 258 | C8 | ORA | D | 50 | −4.424 | −2.718 | −29.006 | 1.00 | 22.12 | C |
| HETATM | 259 | C9 | ORA | D | 50 | −2.471 | −4.075 | −29.273 | 1.00 | 23.98 | C |
| HETATM | 260 | C10 | ORA | D | 50 | −3.638 | −1.671 | −28.528 | 1.00 | 20.57 | C |
| HETATM | 261 | C11 | ORA | D | 50 | −2.242 | −1.800 | −28.406 | 1.00 | 19.21 | C |
| HETATM | 262 | C12 | ORA | D | 50 | 1.582 | −0.118 | −26.071 | 1.00 | 17.67 | C |
| HETATM | 263 | C13 | ORA | D | 50 | −1.493 | −0.640 | −27.883 | 1.00 | 19.08 | C |
| HETATM | 264 | C14 | ORA | D | 50 | −2.288 | 0.559 | −27.544 | 1.00 | 21.57 | C |
| HETATM | 265 | C15 | ORA | D | 50 | −3.845 | −3.925 | −29.382 | 1.00 | 29.21 | C |
| HETATM | 266 | C16 | ORA | D | 50 | −1.650 | −3.048 | −28.798 | 1.00 | 22.97 | C |
| HETATM | 267 | O | HOH | A | 7 | 4.021 | 1.708 | −3.065 | 1.00 | 22.30 | O |
| HETATM | 268 | O | HOH | B | 7 | −0.638 | 3.642 | −5.927 | 1.00 | 27.52 | O |
| HETATM | 269 | O | HOH | B | 8 | −6.045 | 0.636 | 1.698 | 1.00 | 17.78 | O |
| HETATM | 270 | O | HOH | B | 9 | −7.490 | 1.687 | 3.613 | 1.00 | 30.34 | O |
| HETATM | 271 | O | HOH | B | 10 | −5.082 | −2.069 | 0.006 | 1.00 | 22.65 | O |
| HETATM | 272 | O | HOH | B | 11 | −0.570 | 5.965 | −9.442 | 1.00 | 23.16 | O |
| HETATM | 273 | O | HOH | B | 12 | −0.718 | −2.672 | 0.888 | 1.00 | 22.89 | O |
| HETATM | 274 | O | HOH | B | 13 | −0.759 | 0.038 | 2.468 | 1.00 | 24.46 | O |
| HETATM | 275 | O | HOH | B | 14 | −3.192 | −3.980 | −0.075 | 1.00 | 24.91 | O |
| HETATM | 276 | O | HOH | C | 10 | 3.340 | −8.796 | −28.419 | 1.00 | 31.96 | O |
| HETATM | 277 | O | HOH | D | 7 | −2.729 | 7.940 | −19.042 | 1.00 | 31.84 | O |

| | | | | | |
|---|---|---|---|---|---|
| CONECT | 213 | 217 | 221 | | |
| CONECT | 214 | 218 | 235 | | |
| CONECT | 215 | 216 | | | |
| CONECT | 216 | 215 | 222 | 224 | 238 |
| CONECT | 217 | 213 | 225 | | |
| CONECT | 218 | 214 | 236 | | |
| CONECT | 219 | 220 | | | |
| CONECT | 220 | 219 | 226 | 228 | 239 |
| CONECT | 221 | 213 | 227 | | |
| CONECT | 222 | 216 | | | |
| CONECT | 223 | 229 | 233 | | |
| CONECT | 224 | 216 | | | |
| CONECT | 225 | 217 | 235 | | |
| CONECT | 226 | 220 | | | |
| CONECT | 227 | 221 | 235 | | |
| CONECT | 228 | 220 | | | |
| CONECT | 229 | 223 | 237 | | |
| CONECT | 230 | 237 | | | |
| CONECT | 231 | 233 | 238 | | |
| CONECT | 232 | 238 | 239 | | |
| CONECT | 233 | 223 | 231 | 234 | |
| CONECT | 234 | 233 | 236 | 239 | |
| CONECT | 235 | 214 | 225 | 227 | |
| CONECT | 236 | 218 | 234 | 237 | |
| CONECT | 237 | 229 | 230 | 236 | |
| CONECT | 238 | 216 | 231 | 232 | |
| CONECT | 239 | 220 | 232 | 234 | |
| CONECT | 240 | 244 | 248 | | |
| CONECT | 241 | 245 | 262 | | |
| CONECT | 242 | 243 | | | |
| CONECT | 243 | 242 | 249 | 251 | 265 |
| CONECT | 244 | 240 | 252 | | |
| CONECT | 245 | 241 | 263 | | |
| CONECT | 246 | 247 | | | |
| CONECT | 247 | 246 | 253 | 255 | 266 |
| CONECT | 248 | 240 | 254 | | |
| CONECT | 249 | 243 | | | |
| CONECT | 250 | 256 | 260 | | |
| CONECT | 251 | 243 | | | |
| CONECT | 252 | 244 | 262 | | |
| CONECT | 253 | 247 | | | |
| CONECT | 254 | 248 | 262 | | |
| CONECT | 255 | 247 | | | |
| CONECT | 256 | 250 | 264 | | |
| CONECT | 257 | 264 | | | |
| CONECT | 258 | 260 | 265 | | |
| CONECT | 259 | 265 | 266 | | |
| CONECT | 260 | 250 | 258 | 261 | |
| CONECT | 261 | 260 | 263 | 266 | |
| CONECT | 262 | 241 | 252 | 254 | |
| CONECT | 263 | 245 | 261 | 264 | |
| CONECT | 264 | 256 | 257 | 263 | |
| CONECT | 265 | 243 | 258 | 259 | |
| CONECT | 266 | 247 | 259 | 261 | |

| | | | | | | | | | | | | |
|---|---|---|---|---|---|---|---|---|---|---|---|---|
| MASTER | 238 | 0 | 2 | 0 | 4 | 0 | 5 | 6 | 273 | 4 | 54 | 4 |
| END | | | | | | | | | | | | |

TABLE 4
Atomic coordinates of structure of an amyloid-forming peptide VQIVYK
(SEQ ID NO: 2) from the tau protein in complex with Orange G

| | | | |
|---|---|---|---|
| HEADER | PROTEIN FIBRIL | 16-SEP-10 | 3OVL |

| | |
|---|---|
| TITLE | STRUCTURE OF AN AMYLOID FORMING PEPTIDE VQIVYK FROM THE TAU PROTEIN IN |

| | | |
|---|---|---|
| TITLE | 2 | COMPLEX WITH ORANGE G |

| | |
|---|---|
| COMPND | MOL_ID: 1; |

| | | |
|---|---|---|
| COMPND | 2 | MOLECULE: MICROTUBULE-ASSOCIATED PROTEIN; |
| COMPND | 3 | CHAIN: A; |
| COMPND | 4 | FRAGMENT: VQIVYK (RESIDUES 306-311); |
| COMPND | 5 | ENGINEERED: YES |

| | |
|---|---|
| SOURCE | MOL_ID: 1; |

| | | |
|---|---|---|
| SOURCE | 2 | SYNTHETIC: YES; |
| SOURCE | 3 | OTHER_DETAILS: VQIVYK (RESIDUES 306-311) FROM TAU, SYNTHESIZED |

| | |
|---|---|
| KEYWDS | AMYLOID-LIKE PROTOFIBRIL IN COMPLEX WITH A SMALL-MOLECULE BINDER, |

| | | |
|---|---|---|
| KEYWDS | 2 | PROTEIN FIBRIL |

| | |
|---|---|
| EXPDTA | X-RAY DIFFRACTION |
| AUTHOR | M. LANDAU, D. EISENBERG |

| | | | |
|---|---|---|---|
| REVDAT | 1 | 06-JUL-11 3OVL | 0 |

| JRNL | AUTH | | M. LANDAU, M. R. SAWAYA, K. F. FAULL, A. LAGANOWSKY, L. JIANG, |
|---|---|---|---|
| JRNL | AUTH | 2 | S. A. SIEVERS, J. LIU, J. R. BARRIO, D. EISENBERG |

| JRNL | TITL | TOWARDS A PHARMACOPHORE FOR AMYLOID. | | |
|---|---|---|---|---|
| JRNL | REF | PLOS BIOL. | V. | 9 E1001 2011 |

| | | |
|---|---|---|
| JRNL | REFN | ISSN 1544-9173 |

| | | |
|---|---|---|
| JRNL | PMID | 21695112 |
| JRNL | DOI | 10.1371/JOURNAL. PBIO.1001080 |

| REMARK | 2 | | |
|---|---|---|---|
| REMARK | 2 | RESOLUTION. | 1.81 ANGSTROMS. |
| REMARK | 3 | | |

| | | |
|---|---|---|
| REMARK | 3 | REFINEMENT. |

| | | | |
|---|---|---|---|
| REMARK | 3 | PROGRAM | : REFMAC 5.5.0109 |
| REMARK | 3 | AUTHORS | : MURSHUDOV, VAGIN, DODSON |

| REMARK | 3 | |
|---|---|---|
| REMARK | 3 | REFINEMENT TARGET : MAXIMUM LIKELIHOOD |

| REMARK | 3 | |
|---|---|---|
| REMARK | 3 | DATA USED IN REFINEMENT. |

| | | | |
|---|---|---|---|
| REMARK | 3 | RESOLUTION RANGE HIGH (ANGSTROMS) | : 1.81 |
| REMARK | 3 | RESOLUTION RANGE LOW (ANGSTROMS) | : 26.82 |
| REMARK | 3 | DATA CUTOFF (SIGMA(F)) | : 0.000 |
| REMARK | 3 | COMPLETENESS FOR RANGE (%) | : 88.0 |
| REMARK | 3 | NUMBER OF REFLECTIONS | : 586 |

| REMARK | 3 | |
|---|---|---|
| REMARK | 3 | FIT TO DATA USED IN REFINEMENT. |

| | | | |
|---|---|---|---|
| REMARK | 3 | CROSS-VALIDATION METHOD | : THROUGHOUT |
| REMARK | 3 | FREE R VALUE TEST SET SELECTION | : RANDOM |
| REMARK | 3 | R VALUE (WORKING + TEST SET) | : 0.259 |
| REMARK | 3 | R VALUE (WORKING SET) | : 0.259 |
| REMARK | 3 | FREE R VALUE | : 0.259 |
| REMARK | 3 | FREE R VALUE TEST SET SIZE (%) | : 10.400 |
| REMARK | 3 | FREE R VALUE TEST SET COUNT | : 61 |

| REMARK | 3 | |
|---|---|---|
| REMARK | 3 | FIT IN THE HIGHEST RESOLUTION BIN. |

| | | | |
|---|---|---|---|
| REMARK | 3 | TOTAL NUMBER OF BINS USED | : 5 |
| REMARK | 3 | BIN RESOLUTION RANGE HIGH (A) | : 1.80 |
| REMARK | 3 | BIN RESOLUTION RANGE LOW (A) | : 2.02 |
| REMARK | 3 | REFLECTION IN BIN (WORKING SET) | : 118 |
| REMARK | 3 | BIN COMPLETENESS (WORKING + TEST) (%) | : 76.37 |
| REMARK | 3 | BIN R VALUE (WORKING SET) | : 0.3770 |
| REMARK | 3 | BIN FREE R VALUE SET COUNT | : 21 |
| REMARK | 3 | BIN FREE R VALUE | : 0.4480 |

| REMARK | 3 | |
|---|---|---|
| REMARK | 3 | NUMBER OF NON-HYDROGEN ATOMS USED IN REFINEMENT. |

| | | | |
|---|---|---|---|
| REMARK | 3 | PROTEIN ATOMS | : 53 |
| REMARK | 3 | NUCLEIC ACID ATOMS | : 0 |
| REMARK | 3 | HETEROGEN ATOMS | : 33 |
| REMARK | 3 | SOLVENT ATOMS | : 2 |

| REMARK | 3 | |
|---|---|---|
| REMARK | 3 | B VALUES. |

| | | | |
|---|---|---|---|
| REMARK | 3 | FROM WILSON PLOT (A**2) | : 19.90 |
| REMARK | 3 | MEAN B VALUE (OVERALL. A**2) | : 20.28 |

| | | |
|---|---|---|
| REMARK | 3 | OVERALL ANISOTROPIC B VALUE. |

| | | | |
|---|---|---|---|
| REMARK | 3 | B11 (A**2): | −0.66000 |
| REMARK | 3 | B22 (A**2): | −0.70000 |
| REMARK | 3 | B33 (A**2): | 1.26000 |
| REMARK | 3 | B12 (A**2): | 0.00000 |
| REMARK | 3 | B13 (A**2): | −0.24000 |
| REMARK | 3 | B23 (A**2): | 0.00000 |

| REMARK | 3 | |
|---|---|---|
| REMARK | 3 | ESTIMATED OVERALL COORDINATE ERROR. |

| | | | |
|---|---|---|---|
| REMARK | 3 | ESU BASED ON R VALUE (A) | : NULL |
| REMARK | 3 | ESU BASED ON FREE R VALUE (A) | : 0.207 |
| REMARK | 3 | ESU BASED ON MAXIMUM LIKELIHOOD (A) | : 0.163 |
| REMARK | 3 | ESU FOR B VALUES BASED ON MAXIMUM LIKELIHOOD (A**2) | : 5.919 |

| REMARK | 3 | |
|---|---|---|
| REMARK | 3 | CORRELATION COEFFICIENTS. |

| | | | |
|---|---|---|---|
| REMARK | 3 | CORRELATION COEFFICIENT FO-FC | : 0.918 |
| REMARK | 3 | CORRELATION COEFFICIENT FO-FC FREE | : 0.964 |

| REMARK | 3 | | | | |
|---|---|---|---|---|---|
| REMARK | 3 | RMS DEVIATIONS FROM IDEAL VALUES | COUNT | RMS | WEIGHT |

| | | | | | |
|---|---|---|---|---|---|
| REMARK | 3 | BOND LENGTHS REFINED ATOMS (A): | 85; | 0.011; | 0.022 |
| REMARK | 3 | BOND LENGTHS OTHERS (A): | 44; | 0.005; | 0.020 |
| REMARK | 3 | BOND ANGLES REFINED ATOMS (DEGREES): | 118; | 1.220; | 2.402 |
| REMARK | 3 | BOND ANGLES OTHERS (DEGREES): | 104; | 0.627; | 3.000 |
| REMARK | 3 | TORSION ANGLES, PERIOD 1 (DEGREES): | 5; | 6.501; | 5.000 |
| REMARK | 3 | TORSION ANGLES, PERIOD 2 (DEGREES): | 2; | 39.537; | 25.000 |
| REMARK | 3 | TORSION ANGLES, PERIOD 3 (DEGREES): | 11; | 8.441; | 15.000 |
| REMARK | 3 | TORSION ANGLES, PERIOD 4 (DEGREES): | NULL; | NULL; | NULL |
| REMARK | 3 | CHIRAL-CENTER RESTRAINTS (A**3): | 11; | 0.080; | 0.200 |
| REMARK | 3 | GENERAL PLANES REFINED ATOMS (A): | 80; | 0.003; | 0.020 |
| REMARK | 3 | GENERAL PLANES OTHERS (A): | 18; | 0.000; | 0.020 |
| REMARK | 3 | NON-BONDED CONTACTS REFINED ATOMS (A): | NULL; | NULL; | NULL |
| REMARK | 3 | NON-BONDED CONTACTS OTHERS (A): | NULL; | NULL; | NULL |
| REMARK | 3 | NON-BONDED TORSION REFINED ATOMS (A): | NULL; | NULL; | NULL |
| REMARK | 3 | NON-BONDED TORSION OTHERS (A): | NULL; | NULL; | NULL |
| REMARK | 3 | H-BOND (X...Y) REFINED ATOMS (A): | NULL; | NULL; | NULL |
| REMARK | 3 | H-BOND (X...Y) OTHERS (A): | NULL; | NULL; | NULL |
| REMARK | 3 | POTENTIAL METAL-ION REFINED ATOMS (A): | NULL; | NULL; | NULL |
| REMARK | 3 | POTENTIAL METAL-ION OTHERS (A): | NULL; | NULL; | NULL |
| REMARK | 3 | SYMMETRY VDW REFINED ATOMS (A): | NULL; | NULL; | NULL |
| REMARK | 3 | SYMMETRY VDW OTHERS (A): | NULL; | NULL; | NULL |
| REMARK | 3 | SYMMETRY H-BOND REFINED ATOMS (A): | NULL; | NULL; | NULL |
| REMARK | 3 | SYMMETRY H-BOND OTHERS (A): | NULL; | NULL; | NULL |
| REMARK | 3 | SYMMETRY METAL-ION REFINED ATOMS (A): | NULL; | NULL; | NULL |
| REMARK | 3 | SYMMETRY METAL-ION OTHERS (A): | NULL; | NULL; | NULL |

| | | | | | |
|---|---|---|---|---|---|
| REMARK | 3 | | | | |
| REMARK | 3 | ISOTROPIC THERMAL FACTOR RESTRAINTS. | COUNT | RMS | WEIGHT |

| | | | | | |
|---|---|---|---|---|---|
| REMARK | 3 | MAIN-CHAIN BOND REFINED ATOMS (A**2): | 33; | 1.089; | 1.500 |
| REMARK | 3 | MAIN-CHAIN BOND OTHER ATOMS (A**2): | 11; | 0.410; | 1.500 |
| REMARK | 3 | MAIN-CHAIN ANGLE REFINED ATOMS (A**2): | 52; | 1.640; | 2.000 |
| REMARK | 3 | SIDE-CHAIN BOND REFINED ATOMS (A**2): | 52; | 2.252; | 3.000 |
| REMARK | 3 | SIDE-CHAIN ANGLE REFINED ATOMS (A**2): | 66; | 3.319; | 4.500 |

| | |
|---|---|
| REMARK | 3 |

| | | | | | |
|---|---|---|---|---|---|
| REMARK | 3 | ANISOTROPIC THERMAL FACTOR RESTRAINTS. | COUNT | RMS | WEIGHT |

| | | | | | |
|---|---|---|---|---|---|
| REMARK | 3 | RIGID-BOND RESTRAINTS (A**2): | NULL; | NULL; | NULL |
| REMARK | 3 | SPHERICITY; FREE ATOMS (A**2): | NULL; | NULL; | NULL |
| REMARK | 3 | SPHERICITY; BONDED ATOMS (A**2): | NULL; | NULL; | NULL |

| REMARK | 3 | |
|---|---|---|
| REMARK | 3 | NCS RESTRAINTS STATISTICS |

| | | |
|---|---|---|
| REMARK | 3 | NUMBER OF DIFFERENT NCS GROUPS: NULL |

| | | |
|---|---|---|
| REMARK | 3 | |
| REMARK | 3 | TLS DETAILS |

| | | |
|---|---|---|
| REMARK | 3 | NUMBER OF TLS GROUPS: NULL |

| REMARK | 3 | |
|---|---|---|
| REMARK | 3 | BULK SOLVENT MODELLING. |

| REMARK | 3 | METHOD USED: MASK | |
|---|---|---|---|
| REMARK | 3 | PARAMETERS FOR MASK CALCULATION | |
| REMARK | 3 | VDW PROBE RADIUS | : 1.40 |
| REMARK | 3 | ION PROBE RADIUS | : 0.80 |
| REMARK | 3 | SHRINKAGE RADIUS | : 0.80 |

| REMARK | 3 | |
|---|---|---|
| REMARK | 3 | OTHER REFINEMENT REMARKS: HYDROGENS HAVE BEEN ADDED IN THE RIDING |
| REMARK | 3 | POSITIONS U VALUES: REFINED INDIVIDUALLY |

| | | |
|---|---|---|
| REMARK | 4 | |
| REMARK | 4 | 3OVL COMPLIES WITH FORMAT V. 3.20. 01-DEC-08 |
| REMARK | 100 | |
| REMARK | 100 | THIS ENTRY HAS BEEN PROCESSED BY RCSB ON 27-OCT-10. |
| REMARK | 100 | THE RCSB ID CODE IS RCSB061628. |
| REMARK | 200 | |
| REMARK | 200 | EXPERIMENTAL DETAILS |

| | | | |
|---|---|---|---|
| REMARK | 200 | EXPERIMENT TYPE | : X-RAY DIFFRACTION |
| REMARK | 200 | DATE OF DATA COLLECTION | : 17-NOV-08 |
| REMARK | 200 | TEMPERATURE (KELVIN) | : 100 |
| REMARK | 200 | PH | : NULL |
| REMARK | 200 | NUMBER OF CRYSTALS USED | : 1 |
| REMARK | 200 | | |
| REMARK | 200 | SYNCHROTRON (Y/N) | : Y |
| REMARK | 200 | RADIATION SOURCE | : APS |
| REMARK | 200 | BEAMLINE | : 24-ID-E |
| REMARK | 200 | X-RAY GENERATOR MODEL | : NULL |
| REMARK | 200 | MONOCHROMATIC OR LAUE (M/L) | : M |
| REMARK | 200 | WAVELENGTH OR RANGE (A) | : 0.9792 |
| REMARK | 200 | MONOCHROMATOR | : NULL |
| REMARK | 200 | OPTICS | : NULL |
| REMARK | 200 | | |
| REMARK | 200 | DETECTOR TYPE | : CCD |
| REMARK | 200 | DETECTOR MANUFACTURER | : ADSC QUANTUM 315 |
| REMARK | 200 | INTENSITY-INTEGRATION SOFTWARE | : DENZO |
| REMARK | 200 | DATA SCALING SOFTWARE | : SCALEPACK |
| REMARK | 200 | | |
| REMARK | 200 | NUMBER OF UNIQUE REFLECTIONS | : 587 |
| REMARK | 200 | RESOLUTION RANGE HIGH (A) | : 1.800 |
| REMARK | 200 | RESOLUTION RANGE LOW (A) | : 90.000 |
| REMARK | 200 | REJECTION CRITERIA (SIGMA(I)) | : −3.000 |
| REMARK | 200 | | |

| | | |
|---|---|---|
| REMARK | 200 | OVERALL. |

| | | | |
|---|---|---|---|
| REMARK | 200 | COMPLETENESS FOR RANGE (%) | : 87.6 |
| REMARK | 200 | DATA REDUNDANCY | : 2.400 |
| REMARK | 200 | R MERGE (I) | : 0.17900 |
| REMARK | 200 | R SYM (I) | : NULL |
| REMARK | 200 | <I/SIGMA(I)> FOR THE DATA SET | : 4.5000 |
| REMARK | 200 | | |

| | | |
|---|---|---|
| REMARK | 200 | IN THE HIGHEST RESOLUTION SHELL. |

| | | | |
|---|---|---|---|
| REMARK | 200 | HIGHEST RESOLUTION SHELL, RANGE HIGH (A) | : 1.80 |
| REMARK | 200 | HIGHEST RESOLUTION SHELL, RANGE LOW (A) | : 1.94 |

| | | | |
|---|---|---|---|
| REMARK | 200 | COMPLETENESS FOR SHELL (%) | : 71.0 |
| REMARK | 200 | DATA REDUNDANCY IN SHELL | : 1.40 |
| REMARK | 200 | R MERGE FOR SHELL (I) | : 0.43300 |
| REMARK | 200 | R SYM FOR SHELL (I) | : NULL |
| REMARK | 200 | <I/SIGMA(I)> FOR SHELL | : NULL |

| | | |
|---|---|---|
| REMARK | 200 | |
| REMARK | 200 | DIFFRACTION PROTOCOL: SINGLE WAVELENGTH |
| REMARK | 200 | METHOD USED TO DETERMINE THE STRUCTURE: MOLECULAR REPLACEMENT |
| REMARK | 200 | SOFTWARE USED: PHASER |
| REMARK | 200 | STARTING MODEL: NULL |
| REMARK | 200 | |
| REMARK | 200 | REMARK: NULL |
| REMARK | 280 | |
| REMARK | 280 | CRYSTAL |
| REMARK | 280 | SOLVENT CONTENT, VS (%): 35.66 |
| REMARK | 280 | MATTHEWS COEFFICIENT, VM (ANGSTROMS**3/DA): 1.91 |
| REMARK | 280 | |
| REMARK | 280 | CRYSTALLIZATION CONDITIONS: RESERVOIR CONTAINED 0.1M ZINC ACETATE |

| | | |
|---|---|---|
| REMARK | 280 | DIHYDRATE, 18% PEG 3350, VAPOR DIFFUSION, HANGING DROP, |
| REMARK | 280 | TEMPERATURE 291K |

| | | |
|---|---|---|
| REMARK | 290 | |
| REMARK | 290 | CRYSTALLOGRAPHIC SYMMETRY |
| REMARK | 290 | SYMMETRY OPERATORS FOR SPACE GROUP: C 1 2 1 |
| REMARK | 290 | |

| | | | |
|---|---|---|---|
| REMARK | 290 | SYMOP | SYMMETRY |
| REMARK | 290 | NNNMMM | OPERATOR |
| REMARK | 290 | 1555 | X, Y, Z |
| REMARK | 290 | 2555 | −X, Y, −Z |
| REMARK | 290 | 3555 | X + 1/2, Y + 1/2, Z |
| REMARK | 290 | 4555 | −X + 1/2, Y + 1/2, −Z |
| REMARK | 290 | | |

| | | | |
|---|---|---|---|
| REMARK | 290 | WHERE | NNN −> OPERATOR NUMBER |

| | | |
|---|---|---|
| REMARK | 290 | MMM −> TRANSLATION VECTOR |

| REMARK | 290 | |
|---|---|---|
| REMARK | 290 | CRYSTALLOGRAPHIC SYMMETRY TRANSFORMATIONS |
| REMARK | 290 | THE FOLLOWING TRANSFORMATIONS OPERATE ON THE ATOM/HETATM |
| REMARK | 290 | RECORDS IN THIS ENTRY TO PRODUCE CRYSTALLOGRAPHICALLY |
| REMARK | 290 | RELATED MOLECULES. |

| | | | | | | | |
|---|---|---|---|---|---|---|---|
| REMARK | 290 | SMTRY1 | 1 | 1.000000 | 0.000000 | 0.000000 | 0.00000 |
| REMARK | 290 | SMTRY2 | 1 | 0.000000 | 1.000000 | 0.000000 | 0.00000 |
| REMARK | 290 | SMTRY3 | 1 | 0.000000 | 0.000000 | 1.000000 | 0.00000 |
| REMARK | 290 | SMTRY1 | 2 | −1.000000 | 0.000000 | 0.000000 | 0.00000 |
| REMARK | 290 | SMTRY2 | 2 | 0.000000 | 1.000000 | 0.000000 | 0.00000 |
| REMARK | 290 | SMTRY3 | 2 | 0.000000 | 0.000000 | −1.000000 | 0.00000 |
| REMARK | 290 | SMTRY1 | 3 | 1.000000 | 0.000000 | 0.000000 | 27.52800 |
| REMARK | 290 | SMTRY2 | 3 | 0.000000 | 1.000000 | 0.000000 | 2.41550 |
| REMARK | 290 | SMTRY3 | 3 | 0.000000 | 0.000000 | 1.000000 | 0.00000 |
| REMARK | 290 | SMTRY1 | 4 | −1.000000 | 0.000000 | 0.000000 | 27.52800 |
| REMARK | 290 | SMTRY2 | 4 | 0.000000 | 1.000000 | 0.000000 | 2.41550 |
| REMARK | 290 | SMTRY3 | 4 | 0.000000 | 0.000000 | −1.000000 | 0.00000 |
| REMARK | 290 | | | | | | |

| | | |
|---|---|---|
| REMARK | 290 | REMARK: NULL |
| REMARK | 300 | |
| REMARK | 300 | BIOMOLECULE: 1 |
| REMARK | 300 | SEE REMARK 350 FOR THE AUTHOR PROVIDED AND/OR PROGRAM |
| REMARK | 300 | GENERATED ASSEMBLY INFORMATION FOR THE STRUCTURE IN |
| REMARK | 300 | THIS ENTRY. THE REMARK MAY ALSO PROVIDE INFORMATION ON |
| REMARK | 300 | BURIED SURFACE AREA. |
| REMARK | 300 | REMARK: THE BIOLOGICAL UNIT IS A PAIR OF BETA SHEETS WITH ORANGE G |
| REMARK | 300 | INTERRELATING BETWEEN TWO PAIRS OF SHEETS. ONE SHEET IS CONSTRUCTED |
| REMARK | 300 | FROM CHAIN A AND UNIT CELL TRANSLATIONS ALONG THE B DIRECTION (I.E. |
| REMARK | 300 | X, Y + 1, Z; X, Y + 2, Z; X, Y + 3, Z, ETC.). THE SECOND SHEET IS CONSTRUCTED |
| REMARK | 300 | FROM −X − 1/2, 1/2 + Y, −Z; −X − 1/2, 3/2 + Y, −Z; −X − 1/2, 5/2 + Y, −Z; ETC. THE |
| REMARK | 300 | OTHER PAIRS OF SHEETS WILL BE CONTRACTED FROM −X, Y, −Z + 1, −X, Y + 1, −Z + |
| REMARK | 300 | 1,−X, Y + 2, −Z + 1; ETC. AND FROM X + 1/2, 1/2 + Y, Z + 1, X + 1/2, 3/2 + Y, Z + 1, X + 1/ |
| REMARK | 300 | 2, 5/2 + Y, Z + 1; ETC. |
| REMARK | 350 | |
| REMARK | 350 | COORDINATES FOR A COMPLETE MULTIMER REPRESENTING THE KNOWN |
| REMARK | 350 | BIOLOGICALLY SIGNIFICANT OLIGOMERIZATION STATE OF THE |
| REMARK | 350 | MOLECULE CAN BE GENERATED BY APPLYING BIOMT TRANSFORMATIONS |
| REMARK | 350 | GIVEN BELOW. BOTH NON-CRYSTALLOGRAPHIC AND |
| REMARK | 350 | CRYSTALLOGRAPHIC OPERATIONS ARE GIVEN. |
| REMARK | 350 | |
| REMARK | 350 | BIOMOLECULE: 1 |
| REMARK | 350 | AUTHOR DETERMINED BIOLOGICAL UNIT: HEXAMERIC |
| REMARK | 350 | APPLY THE FOLLOWING TO CHAINS: A |

| | | | | | | | |
|---|---|---|---|---|---|---|---|
| REMARK | 350 | BIOMT1 | 1 | 1.000000 | 0.000000 | 0.000000 | 0.00000 |
| REMARK | 350 | BIOMT2 | 1 | 0.000000 | 1.000000 | 0.000000 | 0.00000 |
| REMARK | 350 | BIOMT3 | 1 | 0.000000 | 0.000000 | 1.000000 | 0.00000 |
| REMARK | 350 | BIOMT1 | 2 | 1.000000 | 0.000000 | 0.000000 | 0.00000 |
| REMARK | 350 | BIOMT2 | 2 | 0.000000 | 1.000000 | 0.000000 | 4.83100 |
| REMARK | 350 | BIOMT3 | 2 | 0.000000 | 0.000000 | 1.000000 | 0.00000 |
| REMARK | 350 | BIOMT1 | 3 | 1.000000 | 0.000000 | 0.000000 | 0.00000 |
| REMARK | 350 | BIOMT2 | 3 | 0.000000 | 1.000000 | 0.000000 | 9.66200 |
| REMARK | 350 | BIOMT3 | 3 | 0.000000 | 0.000000 | 1.000000 | 0.00000 |
| REMARK | 350 | BIOMT1 | 4 | −1.000000 | 0.000000 | 0.000000 | −27.52800 |
| REMARK | 350 | BIOMT2 | 4 | 0.000000 | 1.000000 | 0.000000 | 2.41550 |
| REMARK | 350 | BIOMT3 | 4 | 0.000000 | 0.000000 | −1.000000 | 0.00000 |
| REMARK | 350 | BIOMT1 | 5 | −1.000000 | 0.000000 | 0.000000 | −27.52800 |
| REMARK | 350 | BIOMT2 | 5 | 0.000000 | 1.000000 | 0.000000 | 7.24650 |
| REMARK | 350 | BIOMT3 | 5 | 0.000000 | 0.000000 | −1.000000 | 0.00000 |
| REMARK | 350 | BIOMT1 | 6 | −1.000000 | 0.000000 | 0.000000 | −27.52800 |
| REMARK | 350 | BIOMT2 | 6 | 0.000000 | 1.000000 | 0.000000 | 12.07750 |
| REMARK | 350 | BIOMT3 | 6 | 0.000000 | 0.000000 | −1.000000 | 0.00000 |

| REMARK | 375 | | | | |
|---|---|---|---|---|---|
| REMARK | 375 | SPECIAL POSITION | | | |
| REMARK | 375 | THE FOLLOWING ATOMS ARE FOUND TO BE WITHIN 0.15 ANGSTROMS | | | |
| REMARK | 375 | OF A SYMMETRY RELATED ATOM AND ARE ASSUMED TO BE ON SPECIAL | | | |
| REMARK | 375 | POSITIONS. | | | |
| REMARK | 375 | | | | |
| REMARK | 375 | ATOM RES CSSEQI | | | |
| REMARK | 375 | ZN | ZN A | 7 | LIES ON A SPECIAL POSITION. |

| REMARK | 620 | | | | | |
|---|---|---|---|---|---|---|
| REMARK | 620 | METAL COORDINATION | | | | |
| REMARK | 620 | (M = MODEL NUMBER; RES = RESIDUE NAME; C = CHAIN IDENTIFIER; | | | | |
| REMARK | 620 | SSEQ = SEQUENCE NUMBER; I = INSERTION CODE) : | | | | |
| REMARK | 620 | | | | | |
| REMARK | 620 | COORDINATION ANGLES FOR: | M | RES | CSSEQI | METAL |

| | | | | |
|---|---|---|---|---|
| REMARK | 620 | ZN A | 8 | ZN |

| | | | | | |
|---|---|---|---|---|---|
| REMARK | 620 | N | RES | CSSEQI | ATOM |

| | | | | | | |
|---|---|---|---|---|---|---|
| REMARK | 620 | 1 | ACY A | 9 | OXT | |
| REMARK | 620 | 2 | LYS A | 6 | O | 103.0 |
| REMARK | 620 | N | | | | 1 |

| | | | | | | |
|---|---|---|---|---|---|---|
| REMARK | 620 | | | | | |
| REMARK | 620 | COORDINATION ANGLES FOR: | M | RES | CSSEQI | METAL |

| | | | | |
|---|---|---|---|---|
| REMARK | 620 | ZN A | 7 | ZN |

| | | | | | |
|---|---|---|---|---|---|
| REMARK | 620 | N | RES | CSSEQI | ATOM |

| | | | | | | | |
|---|---|---|---|---|---|---|---|
| REMARK | 620 | 1 | ORA A | 79 | O4 | | |
| REMARK | 620 | 2 | LYS A | 6 | NZ | 89.8 | |
| REMARK | 620 | 3 | ORA A | 79 | S1 | 30.0 | 111.1 |
| REMARK | 620 | N | | | | 1 | 2 |

| REMARK | 800 | | |
|---|---|---|---|
| REMARK | 800 | SITE | |
| REMARK | 800 | SITE_IDENTIFIER: AC1 | |
| REMARK | 800 | EVIDENCE_CODE: SOFTWARE | |
| REMARK | 800 | SITE_DESCRIPTION: BINDING SITE FOR RESIDUE ZN A 7 | |
| REMARK | 800 | | |
| REMARK | 800 | SITE_IDENTIFIER: AC2 | |
| REMARK | 800 | EVIDENCE_CODE: SOFTWARE | |
| REMARK | 800 | SITE_DESCRIPTION: BINDING SITE FOR RESIDUE ZN A 8 | |
| REMARK | 800 | | |
| REMARK | 800 | SITE_IDENTIFIER: AC3 | |
| REMARK | 800 | EVIDENCE_CODE: SOFTWARE | |
| REMARK | 800 | SITE_DESCRIPTION: BINDING SITE FOR RESIDUE ACY A 9 | |
| REMARK | 800 | | |
| REMARK | 800 | SITE_IDENTIFIER: AC4 | |
| REMARK | 800 | EVIDENCE_CODE : SOFTWARE | |
| REMARK | 800 | SITE_DESCRIPTION: BINDING SITE FOR RESIDUE ORA A 79 | |
| REMARK | 900 | | |
| REMARK | 900 | RELATED ENTRIES | |
| REMARK | 900 | RELATED ID: 2ON9 | RELATED DB: PDB |
| REMARK | 900 | APO VQIVYK | |
| REMARK | 900 | RELATED ID: 3FQP | RELATED DB: PDB |

| | | |
|---|---|---|
| REMARK | 900 | APO VQ1VYK (ALTERNATE POLYMORPH) |

| | | | | | | | | | |
|---|---|---|---|---|---|---|---|---|---|
| DBREF | 3OVL | A | 1 | 6 | UNP | P10636 | TAU_HUMAN | 623 | 628 |

| | | | | |
|---|---|---|---|---|
| SEQRES | 1 | A | 6 | VAL GLN ILE VAL TYR LYS |

| | | | | |
|---|---|---|---|---|
| HET | ZN | A | 7 | 1 |
| HET | ZN | A | 8 | 1 |
| HET | ACY | A | 9 | 4 |
| HET | ORA | A | 79 | 27 |

| | |
|---|---|
| HETNAM | ZN ZINC ION |

| | | |
|---|---|---|
| HETNAM | | ACY ACETIC ACID |
| HETNAM | | ORA 7-HYDROXY-8-[(E)-PHENYLDIAZENYL]NAPHTHALENE-1,3- |
| HETNAM | 2 | ORA DISOLFONIC ACID |
| HETSYN | | ORA ORANGE G |

| | | | |
|---|---|---|---|
| FORMOL | 2 | ZN | 2(ZN 2+) |
| FORMOL | 4 | ACY | C2 H4 O2 |
| FORMUL | 5 | ORA | C16 H12 N2 O7 S2 |
| FORMOL | 6 | HOH | *2(H2 O) |

| | | | | | | | | | |
|---|---|---|---|---|---|---|---|---|---|
| LINK | ZN | ZN A | 8 | OXT | ACY A | 9 | 1555 | 1555 | 1.88 |
| LINK | O | LYS A | 6 | ZN | ZN A | 8 | 1555 | 1555 | 2.01 |
| LINK | ZN | ZN A | 7 | O4 | ORA A | 79 | 1555 | 1555 | 2.26 |
| LINK | NZ | LYS A | 6 | ZN | ZN A | 7 | 1555 | 1555 | 2.69 |
| LINK | ZN | ZN A | 7 | S1 | ORA A | 79 | 1555 | 1555 | 2.90 |

| | | | | | | | | | | | |
|---|---|---|---|---|---|---|---|---|---|---|---|
| SITE | 1 | AC1 | 2 | LYS A | 6 | ORA A | 79 | | | | |
| SITE | 1 | AC2 | 3 | VAL A | 1 | LYS A | 6 | ACY A | 9 | | |
| SITE | 1 | AC3 | 3 | VAL A | 1 | LYS A | 6 | ZN A | 8 | | |
| SITE | 1 | AC4 | 4 | GLN A | 2 | VAL A | 4 | LYS A | 6 | ZN A | 7 |

| | | | | | | | | |
|---|---|---|---|---|---|---|---|---|
| CRYST1 | 55.056 | 4.831 | 22.127 | 90.00 | 102.98 | 90.00 | C 1 2 1 | 4 |

| | | | | |
|---|---|---|---|---|
| ORIGX1 | 1.000000 | 0.000000 | 0.000000 | 0.00000 |
| ORIGX2 | 0.000000 | 1.000000 | 0.000000 | 0.00000 |
| ORIGX3 | 0.000000 | 0.000000 | 1.000000 | 0.00000 |
| SCALE1 | 0.018163 | 0.000000 | 0.004187 | 0.00000 |
| SCALE2 | 0.000000 | 0.206996 | 0.000000 | 0.00000 |
| SCALE3 | 0.000000 | 0.000000 | 0.046379 | 0.00000 |

| | | | | | | | | | | |
|---|---|---|---|---|---|---|---|---|---|---|
| ATOM | 1 | N | VAL A | 1 | −7.955 | 0.074 | −3.743 | 1.00 | 13.14 | N |
| ATOM | 2 | CA | VAL A | 1 | −8.833 | 0.532 | −2.629 | 1.00 | 13.01 | C |
| ATOM | 3 | C | VAL A | 1 | −8.405 | −0.121 | −1.289 | 1.00 | 13.33 | C |
| ATOM | 4 | O | VAL A | 1 | −8.354 | −1.351 | −1.185 | 1.00 | 15.52 | O |
| ATOM | 5 | CB | VAL A | 1 | −10.319 | 0.219 | −2.957 | 1.00 | 12.07 | C |
| ATOM | 6 | CG1 | VAL A | 1 | −11.240 | 0.586 | −1.817 | 1.00 | 12.46 | C |
| ATOM | 7 | CG2 | VAL A | 1 | −10.730 | 0.946 | −4.188 | 1.00 | 10.08 | C |
| ATOM | 8 | N | GLN A | 2 | −8.100 | 0.695 | −0.275 | 1.00 | 13.68 | N |
| ATOM | 9 | CA | GLN A | 2 | −7.886 | 0.194 | 1.106 | 1.00 | 12.23 | C |
| ATOM | 10 | C | GLN A | 2 | −8.849 | 0.840 | 2.110 | 1.00 | 12.15 | C |
| ATOM | 11 | O | GLN A | 2 | −8.869 | 2.069 | 2.234 | 1.00 | 11.46 | O |
| ATOM | 12 | CB | GLN A | 2 | −6.443 | 0.445 | 1.560 | 1.00 | 13.05 | C |
| ATOM | 13 | CG | GLN A | 2 | −6.152 | −0.022 | 2.989 | 1.00 | 13.48 | C |
| ATOM | 14 | CD | GLN A | 2 | −4.743 | 0.309 | 3.443 | 1.00 | 19.26 | C |
| ATOM | 15 | OE1 | GLN A | 2 | −3.981 | −0.573 | 3.859 | 1.00 | 21.85 | O |
| ATOM | 16 | NE2 | GLN A | 2 | −4.388 | 1.583 | 3.375 | 1.00 | 22.86 | N |
| ATOM | 17 | N | ILE A | 3 | −9.611 | 0.013 | 2.836 | 1.00 | 11.10 | N |
| ATOM | 18 | CA | ILE A | 3 | −10.513 | 0.474 | 3.908 | 1.00 | 11.69 | C |
| ATOM | 19 | C | ILE A | 3 | −10.130 | −0.183 | 5.236 | 1.00 | 10.68 | C |
| ATOM | 20 | O | ILE A | 3 | −9.963 | −1.403 | 5.316 | 1.00 | 12.60 | O |
| ATOM | 21 | CB | ILE A | 3 | −12.015 | 0.149 | 3.601 | 1.00 | 13.06 | C |
| ATOM | 22 | CG1 | ILE A | 3 | −12.404 | 0.574 | 2.181 | 1.00 | 14.22 | C |
| ATOM | 23 | CG2 | ILE A | 3 | −12.958 | 0.800 | 4.615 | 1.00 | 8.87 | C |
| ATOM | 24 | CD1 | ILE A | 3 | −12.130 | 2.011 | 1.868 | 1.00 | 18.25 | C |
| ATOM | 25 | N | VAL A | 4 | −10.009 | 0.629 | 6.283 | 1.00 | 10.79 | N |
| ATOM | 26 | CA | VAL A | 4 | −9.530 | 0.166 | 7.577 | 1.00 | 9.20 | C |
| ATOM | 27 | C | VAL A | 4 | −10.409 | 0.708 | 8.701 | 1.00 | 10.71 | C |
| ATOM | 28 | O | VAL A | 4 | −10.548 | 1.926 | 8.848 | 1.00 | 11.39 | O |
| ATOM | 29 | CB | VAL A | 4 | −8.090 | 0.680 | 7.852 | 1.00 | 10.48 | C |
| ATOM | 30 | CG1 | VAL A | 4 | −7.661 | 0.311 | 9.265 | 1.00 | 6.30 | C |
| ATOM | 31 | CG2 | VAL A | 4 | −7.095 | 0.152 | 6.814 | 1.00 | 6.87 | C |
| ATOM | 32 | N | TYR A | 5 | −10.987 | −0.193 | 9.491 | 1.00 | 10.21 | N |
| ATOM | 33 | CA | TYR A | 5 | −11.724 | 0.177 | 10.696 | 1.00 | 10.92 | C |
| ATOM | 34 | C | TYR A | 5 | −10.910 | −0.252 | 11.895 | 1.00 | 11.54 | C |
| ATOM | 35 | O | TYR A | 5 | −10.608 | −1.441 | 12.036 | 1.00 | 11.89 | O |
| ATOM | 36 | CB | TYR A | 5 | −13.046 | −0.576 | 10.762 | 1.00 | 10.78 | C |
| ATOM | 37 | CG | TYR A | 5 | −14.058 | −0.185 | 9.731 | 1.00 | 10.88 | C |
| ATOM | 38 | CD1 | TYR A | 5 | −14.994 | 0.802 | 10.003 | 1.00 | 11.13 | C |
| ATOM | 39 | CD2 | TYR A | 5 | −14.095 | −0.812 | 8.484 | 1.00 | 11.11 | C |
| ATOM | 40 | CE1 | TYR A | 5 | −15.944 | 1.167 | 9.058 | 1.00 | 9.38 | C |
| ATOM | 41 | CE2 | TYR A | 5 | −15.055 | −0.458 | 7.528 | 1.00 | 12.71 | C |
| ATOM | 42 | CZ | TYR A | 5 | −15.970 | 0.536 | 7.828 | 1.00 | 14.60 | C |
| ATOM | 43 | OH | TYR A | 5 | −16.919 | 0.911 | 6.911 | 1.00 | 19.28 | O |
| ATOM | 44 | N | LYS A | 6 | −10.596 | 0.691 | 12.777 | 1.00 | 13.21 | N |
| ATOM | 45 | CA | LYS A | 6 | −9.770 | 0.406 | 13.943 | 1.00 | 14.82 | C |
| ATOM | 46 | C | LYS A | 6 | −10.497 | 0.747 | 15.227 | 1.00 | 16.42 | C |
| ATOM | 47 | O | LYS A | 6 | −9.998 | 0.491 | 16.325 | 1.00 | 15.32 | O |
| ATOM | 48 | CB | LYS A | 6 | −8.456 | 1.188 | 13.863 | 1.00 | 16.07 | C |
| ATOM | 49 | CG | LYS A | 6 | −7.546 | 0.731 | 12.738 | 1.00 | 18.03 | C |
| ATOM | 50 | CD | LYS A | 6 | −6.316 | 1.615 | 12.628 | 1.00 | 22.58 | C |
| ATOM | 51 | CE | LYS A | 6 | −5.202 | 0.860 | 11.937 | 1.00 | 25.77 | C |
| ATOM | 52 | NZ | LYS A | 6 | −3.965 | 1.661 | 11.740 | 1.00 | 25.81 | N |
| ATOM | 53 | OXT | LYS A | 6 | −11.597 | 1.299 | 15.201 | 1.00 | 16.38 | O |
| TER | 54 | | LYS A | 6 | | | | | | |
| HETATM | 55 | ZN | ZN A | 7 | −2.485 | −0.373 | 10.781 | 0.50 | 32.05 | ZN |
| HETATM | 56 | ZN | ZN A | 8 | −10.951 | 0.715 | 18.084 | 1.00 | 22.19 | ZN |
| HETATM | 57 | C | ACY A | 9 | −10.116 | 3.224 | 19.245 | 1.00 | 22.09 | C |
| HETATM | 58 | O | ACY A | 9 | −10.028 | 4.464 | 19.224 | 1.00 | 21.97 | O |
| HETATM | 59 | OXT | ACY A | 9 | −10.793 | 2.562 | 18.421 | 1.00 | 20.92 | O |
| HETATM | 60 | CH3 | ACY A | 9 | −9.361 | 2.506 | 20.327 | 1.00 | 23.36 | C |
| HETATM | 61 | C1 | ORA A | 79 | −2.249 | 8.785 | 6.380 | 0.25 | 36.92 | C |
| HETATM | 62 | N1 | ORA A | 79 | −1.432 | 4.790 | 5.471 | 0.25 | 33.27 | N |
| HETATM | 63 | O1 | ORA A | 79 | −5.113 | −2.222 | 7.940 | 0.25 | 33.75 | O |
| HETATM | 64 | S1 | ORA A | 79 | −3.936 | −1.858 | 8.761 | 0.25 | 32.06 | S |
| HETATM | 65 | C2 | ORA A | 79 | −0.995 | 8.446 | 5.839 | 0.25 | 36.47 | C |
| HETATM | 66 | N2 | ORA A | 79 | −1.396 | 3.962 | 6.443 | 0.25 | 32.44 | N |
| HETATM | 67 | O2 | ORA A | 79 | −4.136 | 3.788 | 7.203 | 0.25 | 30.38 | O |
| HETATM | 68 | S2 | ORA A | 79 | −3.242 | 3.196 | 8.224 | 0.25 | 30.16 | S |
| HETATM | 69 | C3 | ORA A | 79 | −3.230 | 7.794 | 6.616 | 0.25 | 36.11 | C |
| HETATM | 70 | O3 | ORA A | 79 | −3.072 | −3.052 | 8.890 | 0.25 | 32.62 | O |
| HETATM | 71 | C4 | ORA A | 79 | −0.566 | −0.169 | 5.524 | 0.25 | 30.05 | C |
| HETATM | 72 | O4 | ORA A | 79 | −4.369 | −1.410 | 10.094 | 0.25 | 29.49 | O |
| HETATM | 73 | C5 | ORA A | 79 | −0.721 | 7.115 | 5.536 | 0.25 | 35.97 | C |
| HETATM | 74 | O5 | ORA A | 79 | −3.956 | 3.153 | 9.520 | 0.25 | 25.49 | O |
| HETATM | 75 | C6 | ORA A | 79 | −2.956 | 6.465 | 6.311 | 0.25 | 35.48 | C |
| HETATM | 76 | O6 | ORA A | 79 | −2.050 | 4.052 | 8.395 | 0.25 | 29.45 | O |
| HETATM | 77 | C7 | ORA A | 79 | 0.137 | 0.829 | 4.867 | 0.25 | 29.92 | C |
| HETATM | 78 | O7 | ORA A | 79 | 0.601 | 3.120 | 4.481 | 0.25 | 28.58 | O |
| HETATM | 79 | C8 | ORA A | 79 | −2.196 | −0.910 | 7.094 | 0.25 | 31.92 | C |
| HETATM | 80 | C9 | ORA A | 79 | −3.479 | 0.652 | 8.388 | 0.25 | 30.72 | C |
| HETATM | 81 | C10 | ORA A | 79 | −1.531 | 0.141 | 6.474 | 0.25 | 30.63 | C |
| HETATM | 82 | C11 | ORA A | 79 | −1.836 | 1.462 | 6.807 | 0.25 | 30.67 | C |
| HETATM | 83 | C12 | ORA A | 79 | −1.703 | 6.128 | 5.771 | 0.25 | 35.15 | C |
| HETATM | 84 | C13 | ORA A | 79 | −1.128 | 2.566 | 6.146 | 0.25 | 31.32 | C |
| HETATM | 85 | C14 | ORA A | 79 | −0.110 | 2.164 | 5.144 | 0.25 | 30.13 | C |
| HETATM | 86 | C15 | ORA A | 79 | −3.167 | −0.656 | 8.052 | 0.25 | 31.46 | C |
| HETATM | 87 | C16 | ORA A | 79 | −2.824 | 1.715 | 7.773 | 0.25 | 30.33 | C |
| HETATM | 88 | O | HOH A | 10 | −13.223 | 2.881 | 15.498 | 1.00 | 28.92 | O |
| HETATM | 89 | O | HOH A | 11 | −13.963 | 0.984 | 14.133 | 1.00 | 27.77 | O |

| | | | | | |
|---|---|---|---|---|---|
| CONECT | 47 | 56 | | | |
| CONECT | 52 | 55 | | | |
| CONECT | 55 | 52 | 64 | 72 | |
| CONECT | 56 | 47 | 59 | | |
| CONECT | 57 | 58 | 59 | 60 | |
| CONECT | 58 | 57 | | | |
| CONECT | 59 | 56 | 57 | | |
| CONECT | 60 | 57 | | | |
| CONECT | 61 | 65 | 69 | | |
| CONECT | 62 | 66 | 83 | | |
| CONECT | 63 | 64 | | | |
| CONECT | 64 | 55 | 63 | 70 | 72 |
| CONECT | 64 | 86 | | | |
| CONECT | 65 | 61 | 73 | | |
| CONECT | 66 | 62 | 84 | | |
| CONECT | 67 | 68 | | | |
| CONECT | 68 | 67 | 74 | 76 | 87 |
| CONECT | 69 | 61 | 75 | | |
| CONECT | 70 | 64 | | | |
| CONECT | 71 | 77 | 81 | | |
| CONECT | 72 | 55 | 64 | | |
| CONECT | 73 | 65 | 83 | | |
| CONECT | 74 | 68 | | | |
| CONECT | 75 | 69 | 83 | | |
| CONECT | 76 | 68 | | | |
| CONECT | 77 | 71 | 85 | | |
| CONECT | 78 | 85 | | | |
| CONECT | 79 | 81 | 86 | | |
| CONECT | 80 | 86 | 87 | | |
| CONECT | 81 | 71 | 79 | 82 | |
| CONECT | 82 | 81 | 84 | 87 | |
| CONECT | 83 | 62 | 73 | 75 | |
| CONECT | 84 | 66 | 82 | 85 | |
| CONECT | 85 | 77 | 78 | 84 | |
| CONECT | 86 | 64 | 79 | 80 | |
| CONECT | 87 | 68 | 80 | 82 | |

| | | | | | | | | | | | | |
|---|---|---|---|---|---|---|---|---|---|---|---|---|
| MASTER | 302 | 0 | 4 | 0 | 0 | 0 | 4 | 6 | 88 | 1 | 36 | 1 |
| END | | | | | | | | | | | | |

TABLE 5
Atomic coordinates of structure of an amyloid-forming peptide VQIVYK
(SEQ ID NO: 2) from the tau protein in complex with Curcumin

HEADER PROTEIN FIBRIL
TITLE STRUCTURE OF AN AMYLOID FORMING PEPTIDE VQIVYK FROM THE TAU PROTEIN IN
TITLE 2 COMPLEX WITH CURCUMIN
AUTHOR M. LANDAU, D. EISENBERG

| REMARK | 3 | |
|---|---|---|
| REMARK | 3 | REFINEMENT. |

| | | | |
|---|---|---|---|
| REMARK | 3 | PROGRAM | : REFMAC 5.5.0109 |
| REMARK | 3 | AUTHORS | : MURSHUDOV, VAGIN, DODSON |

| REMARK | 3 | |
|---|---|---|
| REMARK | 3 | REFINEMENT TARGET: MAXIMUM LIKELIHOOD |

| REMARK | 3 | |
|---|---|---|
| REMARK | 3 | DATA USED IN REFINEMENT. |

| | | | |
|---|---|---|---|
| REMARK | 3 | RESOLUTION RANGE HIGH (ANGSTROMS) | : 1.30 |
| REMARK | 3 | RESOLUTION RANGE LOW (ANGSTROMS) | : 33.52 |
| REMARK | 3 | DATA CUTOFF (SIGMA(F)) | : NONE |
| REMARK | 3 | COMPLETENESS FOR RANGE (%) | : 81.08 |
| REMARK | 3 | NUMBER OF REFLECTIONS | : 945 |

| REMARK | 3 | |
|---|---|---|
| REMARK | 3 | FIT TO DATA USED IN REFINEMENT. |

| | | | |
|---|---|---|---|
| REMARK | 3 | CROSS-VALIDATION METHOD | : THROUGHOUT |
| REMARK | 3 | FREE R VALUE TEST SET SELECTION | : RANDOM |
| REMARK | 3 | R VALUE (WORKING + TEST SET) | : 0.23582 |
| REMARK | 3 | R VALUE (WORKING SET) | : 0.23150 |
| REMARK | 3 | FREE R VALUE | : 0.26886 |
| REMARK | 3 | FREE R VALUE TEST SET SIZE (%) | : 10.3 |
| REMARK | 3 | FREE R VALUE TEST SET COUNT | : 109 |

| REMARK | 3 | |
|---|---|---|
| REMARK | 3 | FIT IN THE HIGHEST RESOLUTION BIN. |

| | | | |
|---|---|---|---|
| REMARK | 3 | TOTAL NUMBER OF BINS USED | : 5 |
| REMARK | 3 | BIN RESOLUTION RANGE HIGH | : 1.302 |
| REMARK | 3 | BIN RESOLUTION RANGE LOW | : 1.456 |
| REMARK | 3 | REFLECTION IN BIN (WORKING SET) | : 186 |
| REMARK | 3 | BIN COMPLETENESS (WORKING + TEST) (%) | : 61.00 |
| REMARK | 3 | BIN R VALUE (WORKING SET) | : 0.481 |
| REMARK | 3 | BIN FREE R VALUE SET COUNT | : 22 |
| REMARK | 3 | BIN FREE R VALUE | : 0.431 |

| REMARK | 3 | |
|---|---|---|
| REMARK | 3 | NUMBER OF NON-HYDROGEN ATOMS USED IN REFINEMENT. |

| | | | |
|---|---|---|---|
| REMARK | 3 | ALL ATOMS | : 60 |

| REMARK | 3 | |
|---|---|---|
| REMARK | 3 | B VALUES. |

| | | | |
|---|---|---|---|
| REMARK | 3 | FROM WILSON PLOT (A**2) | : NULL |
| REMARK | 3 | MEAN B VALUE (OVERALL, A**2) | : 11.033 |

| | | |
|---|---|---|
| REMARK | 3 | OVERALL ANISOTROPIC B VALUE. |

| | | | |
|---|---|---|---|
| REMARK | 3 | B11 (A**2): | 0.58 |
| REMARK | 3 | B22 (A**2): | −1.04 |
| REMARK | 3 | B33 (A**2): | 0.23 |
| REMARK | 3 | B12 (A**2): | 0.00 |
| REMARK | 3 | B13 (A**2): | −0.34 |
| REMARK | 3 | B23 (A**2): | 0.00 |

| REMARK | 3 | |
|---|---|---|
| REMARK | 3 | ESTIMATED OVERALL COORDINATE ERROR. |

| | | | |
|---|---|---|---|
| REMARK | 3 | ESU BASED ON R VALUE (A) | : 0.129 |
| REMARK | 3 | ESU BASED ON FREE R VALUE (A) | : 0.098 |
| REMARK | 3 | ESU BASED ON MAXIMUM LIKELIHOOD (A) | : 0.129 |
| REMARK | 3 | ESU FOR B VALUES BASED ON MAXIMUM LIKELIHOOD (A**2) | : 8.911 |

| REMARK | 3 | |
|---|---|---|
| REMARK | 3 | CORRELATION COEFFICIENTS. |

| | | | |
|---|---|---|---|
| REMARK | 3 | CORRELATION COEFFICIENT FO-FC | : 0.954 |
| REMARK | 3 | CORRELATION COEFFICIENT FO-FC FREE | : 0.893 |

| REMARK | 3 | | | | |
|---|---|---|---|---|---|
| REMARK | 3 | RMS DEVIATIONS FROM IDEAL VALUES | COUNT | RMS | WEIGHT |

| | | | | | |
|---|---|---|---|---|---|
| REMARK | 3 | BOND LENGTHS REFINED ATOMS (A): | 59; | 0.004; | 0.024 |
| REMARK | 3 | BOND LENGTHS OTHERS (A): | 38; | 0.001; | 0.020 |
| REMARK | 3 | BOND ANGLES REFINED ATOMS (DEGREES): | 81; | 0.882; | 2.014 |
| REMARK | 3 | BOND ANGLES OTHERS (DEGREES): | 97; | 0.524; | 3.000 |
| REMARK | 3 | TORSION ANGLES, PERIOD 1 (DEGREES): | 7; | 3.576; | 5.000 |
| REMARK | 3 | TORSION ANGLES, PERIOD 2 (DEGREES): | 2; | 38.702; | 25.000 |
| REMARK | 3 | TORSION ANGLES, PERIOD 3 (DEGREES): | 13; | 8.351; | 15.000 |
| REMARK | 3 | CHIRAL-CENTER RESTRAINTS (A**3): | 11; | 0.053; | 0.200 |
| REMARK | 3 | GENERAL PLANES REFINED ATOMS (A): | 58; | 0.003; | 0.020 |
| REMARK | 3 | GENERAL PLANES OTHERS (A): | 10; | 0.000; | 0.020 |

| | | | | | |
|---|---|---|---|---|---|
| REMARK | 3 | | | | |
| REMARK | 3 | ISOTROPIC THERMAL FACTOR RESTRAINTS. | COUNT | RMS | WEIGHT |

| | | | | | |
|---|---|---|---|---|---|
| REMARK | 3 | MAIN-CHAIN BOND REFINED ATOMS (A**3): | 33; | 1.122; | 1.500 |
| REMARK | 3 | MAIN-CHAIN BOND OTHER ATOMS (A**2): | 12; | 0.446; | 1.500 |
| REMARK | 3 | MAIN-CHAIN ANGLE REFINED ATOMS (A**2): | 57; | 1.603; | 2.000 |
| REMARK | 3 | SIDE-CHAIN BOND REFINED ATOMS (A**2): | 26; | 1.830; | 3.000 |
| REMARK | 3 | SIDE-CHAIN ANGLE REFINED ATOMS (A**2): | 23; | 2.607; | 4.500 |

| | | | | | |
|---|---|---|---|---|---|
| REMARK | 3 | | | | |
| REMARK | 3 | ANISOTROPIC THERMAL FACTOR RESTRAINTS. | COUNT | RMS | WEIGHT |

| | | | | | |
|---|---|---|---|---|---|
| REMARK | 3 | RIGID-BOND RESTRAINTS (A**2): | 97; | 1.021; | 3.000 |

| REMARK | 3 | |
|---|---|---|
| REMARK | 3 | NCS RESTRAINTS STATISTICS |

| | | |
|---|---|---|
| REMARK | 3 | NUMBER OF NCS GROUPS: NULL |

| | | |
|---|---|---|
| REMARK | 3 | |
| REMARK | 3 | TWIN DETAILS |

| | | |
|---|---|---|
| REMARK | 3 | NUMBER OF TWIN DOMAINS: NULL |

| | | |
|---|---|---|
| REMARK | 3 | |
| REMARK | 3 | |
| REMARK | 3 | TLS DETAILS |

| | | |
|---|---|---|
| REMARK | 3 | NUMBER OF TLS GROUPS: NULL |

| REMARK | 3 | |
|---|---|---|
| REMARK | 3 | |
| REMARK | 3 | BULK SOLVENT MODELLING. |

| REMARK | 3 | METHOD USED: NONE | |
|---|---|---|---|
| REMARK | 3 | PARAMETERS FOR MASK CACLULATION | |
| REMARK | 3 | VDW PROBE RADIUS | : NULL |
| REMARK | 3 | ION PROBE RADIUS | : NULL |
| REMARK | 3 | SHRINKAGE RADIUS | : NULL |

| REMARK | 3 | | | | | | |
|---|---|---|---|---|---|---|---|
| REMARK | 3 | OTHER REFINEMENT REMARKS: | | | | | |
| REMARK | 3 | HYDROGENS HAVE BEEN ADDED IN THE RIDING POSITIONS | | | | | |
| REMARK | 3 | U VALUES: REFINED INDIVIDUALLY | | | | | |
| REMARK | 3 | | | | | | |
| CRYST1 | 28.197 | 4.834 | 35.733 | 90.00 | 110.26 | 90.00 | C 1 2 1 |

| | | | | |
|---|---|---|---|---|
| SCALE1 | 0.035465 | 0.000000 | 0.013090 | 0.00000 |
| SCALE2 | 0.000000 | 0.206868 | 0.000000 | 0.00000 |
| SCALE3 | 0.000000 | 0.000000 | 0.029831 | 0.00000 |

| | | | | | | | | | | |
|---|---|---|---|---|---|---|---|---|---|---|
| ATOM | 1 | N | VAL A | 1 | −11.307 | −0.282 | 2.464 | 1.00 | 15.08 | N |

| | | | | | | | | | | | |
|---|---|---|---|---|---|---|---|---|---|---|---|
| ANISOU | 1 | N | VAL A | 1 | 1990 | 1741 | 1999 | 41 | −93 | 83 | N |

| | | | | | | | | | | |
|---|---|---|---|---|---|---|---|---|---|---|
| ATOM | 2 | CA | VAL A | 1 | −9.935 | −0.836 | 2.613 | 1.00 | 14.12 | C |

| | | | | | | | | | | | |
|---|---|---|---|---|---|---|---|---|---|---|---|
| ANISOU | 2 | CA | VAL A | 1 | 1937 | 1625 | 1802 | −8 | −51 | 61 | C |

| | | | | | | | | | | |
|---|---|---|---|---|---|---|---|---|---|---|
| ATOM | 4 | CB | VAL A | 1 | −9.079 | −0.549 | 1.367 | 1.00 | 14.37 | C |

| | | | | | | | | | | | |
|---|---|---|---|---|---|---|---|---|---|---|---|
| ANISOU | 4 | CB | VAL A | 1 | 1991 | 1645 | 1824 | −5 | −51 | 73 | C |

| | | | | | | | | | | |
|---|---|---|---|---|---|---|---|---|---|---|
| ATOM | 6 | CG1 | VAL A | 1 | −7.670 | −1.084 | 1.545 | 1.00 | 15.38 | C |

| | | | | | | | | | | | |
|---|---|---|---|---|---|---|---|---|---|---|---|
| ANISOU | 6 | CG1 | VAL A | 1 | 2100 | 1987 | 1756 | 4 | 23 | 75 | C |

| | | | | | | | | | | |
|---|---|---|---|---|---|---|---|---|---|---|
| ATOM | 10 | CG2 | VAL A | 1 | −9.724 | −1.150 | 0.136 | 1.00 | 14.95 | C |

| | | | | | | | | | | | |
|---|---|---|---|---|---|---|---|---|---|---|---|
| ANISOU | 10 | CG2 | VAL A | 1 | 2112 | 1866 | 1701 | −24 | −129 | 116 | C |

| | | | | | | | | | | |
|---|---|---|---|---|---|---|---|---|---|---|
| ATOM | 14 | C | VAL A | 1 | −9.268 | −0.223 | 3.833 | 1.00 | 13.16 | C |

| | | | | | | | | | | | |
|---|---|---|---|---|---|---|---|---|---|---|---|
| ANISOU | 14 | C | VAL A | 1 | 1790 | 1445 | 1763 | −10 | −33 | 62 | C |

| | | | | | | | | | | |
|---|---|---|---|---|---|---|---|---|---|---|
| ATOM | 15 | O | VAL A | 1 | −9.188 | 0.994 | 3.943 | 1.00 | 14.57 | O |

| | | | | | | | | | | | |
|---|---|---|---|---|---|---|---|---|---|---|---|
| ANISOU | 15 | O | VAL A | 1 | 1982 | 1676 | 1878 | −72 | −61 | 57 | O |

| | | | | | | | | | | |
|---|---|---|---|---|---|---|---|---|---|---|
| ATOM | 19 | N | GLN A | 2 | −8.808 | −1.062 | 4.755 | 1.00 | 12.84 | N |

| | | | | | | | | | | | |
|---|---|---|---|---|---|---|---|---|---|---|---|
| ANISOU | 19 | N | GLN A | 2 | 1737 | 1391 | 1751 | 8 | −29 | 22 | N |

| | | | | | | | | | | |
|---|---|---|---|---|---|---|---|---|---|---|
| ATOM | 20 | CA | GLN A | 2 | −8.080 | −0.588 | 5.926 | 1.00 | 12.37 | C |

| | | | | | | | | | | | |
|---|---|---|---|---|---|---|---|---|---|---|---|
| ANISOU | 20 | CA | GLN A | 2 | 1672 | 1328 | 1699 | 46 | −38 | 1 | C |

| | | | | | | | | | | |
|---|---|---|---|---|---|---|---|---|---|---|
| ATOM | 22 | CB | GLN A | 2 | −8.821 | −0.911 | 7.221 | 1.00 | 12.48 | C |

| | | | | | | | | | | | |
|---|---|---|---|---|---|---|---|---|---|---|---|
| ANISOU | 22 | CB | GLN A | 2 | 1667 | 1321 | 1752 | 67 | −41 | 14 | C |

| | | | | | | | | | | |
|---|---|---|---|---|---|---|---|---|---|---|
| ATOM | 25 | CG | GLN A | 2 | −8.196 | −0.245 | 8.440 | 1.00 | 14.16 | C |

| | | | | | | | | | | | |
|---|---|---|---|---|---|---|---|---|---|---|---|
| ANISOU | 25 | CG | GLN A | 2 | 1714 | 1763 | 1903 | 95 | −129 | 122 | C |

| | | | | | | | | | | |
|---|---|---|---|---|---|---|---|---|---|---|
| ATOM | 28 | CD | GLN A | 2 | −8.804 | −0.696 | 9.742 | 1.00 | 14.97 | C |

| | | | | | | | | | | | |
|---|---|---|---|---|---|---|---|---|---|---|---|
| ANISOU | 28 | CD | GLN A | 2 | 1801 | 2045 | 1840 | 67 | −242 | 197 | C |

| | | | | | | | | | | |
|---|---|---|---|---|---|---|---|---|---|---|
| ATOM | 29 | OE1 | GLN A | 2 | −8.848 | −1.886 | 10.035 | 1.00 | 17.35 | O |

| | | | | | | | | | | | |
|---|---|---|---|---|---|---|---|---|---|---|---|
| ANISOU | 29 | OE1 | GLN A | 2 | 2134 | 2388 | 2066 | 190 | −124 | 276 | O |

| | | | | | | | | | | |
|---|---|---|---|---|---|---|---|---|---|---|
| ATOM | 30 | NE2 | GLN A | 2 | −9.263 | 0.255 | 10.542 | 1.00 | 17.13 | N |

| | | | | | | | | | | | |
|---|---|---|---|---|---|---|---|---|---|---|---|
| ANISOU | 30 | NE2 | GLN A | 2 | 1982 | 2610 | 1915 | 93 | −171 | 107 | N |

| | | | | | | | | | | |
|---|---|---|---|---|---|---|---|---|---|---|
| ATOM | 33 | C | GLN A | 2 | −6.702 | −1.209 | 5.988 | 1.00 | 11.64 | C |

| | | | | | | | | | | | |
|---|---|---|---|---|---|---|---|---|---|---|---|
| ANISOU | 33 | C | GLN A | 2 | 1582 | 1286 | 1555 | 12 | −70 | 0 | C |

| | | | | | | | | | | |
|---|---|---|---|---|---|---|---|---|---|---|
| ATOM | 34 | O | GLN A | 2 | −6.570 | −2.425 | 5.977 | 1.00 | 13.79 | O |

| | | | | | | | | | | | |
|---|---|---|---|---|---|---|---|---|---|---|---|
| ANISOU | 34 | O | GLN A | 2 | 1772 | 1565 | 1900 | 87 | −72 | −6 | O |

| | | | | | | | | | | |
|---|---|---|---|---|---|---|---|---|---|---|
| ATOM | 36 | N | ILE A | 3 | −5.684 | −0.362 | 6.059 | 1.00 | 11.93 | N |

| | | | | | | | | | | | |
|---|---|---|---|---|---|---|---|---|---|---|---|
| ANISOU | 36 | N | ILE A | 3 | 1661 | 1312 | 1560 | 39 | −72 | −46 | N |

| | | | | | | | | | | |
|---|---|---|---|---|---|---|---|---|---|---|
| ATOM | 37 | CA | AILE A | 3 | −4.313 | −0.813 | 6.249 | 0.50 | 11.59 | C |

| | | | | | | | | | | | |
|---|---|---|---|---|---|---|---|---|---|---|---|
| ANISOU | 37 | CA | AILE A | 3 | 1605 | 1305 | 1491 | 16 | −74 | −78 | C |

| | | | | | | | | | | |
|---|---|---|---|---|---|---|---|---|---|---|
| ATOM | 38 | CA | BILE A | 3 | −4.305 | −0.802 | 6.240 | 0.50 | 11.72 | C |

| | | | | | | | | | | | |
|---|---|---|---|---|---|---|---|---|---|---|---|
| ANISOU | 38 | CA | BILE A | 3 | 1626 | 1328 | 1499 | 23 | −69 | −70 | C |

| | | | | | | | | | | |
|---|---|---|---|---|---|---|---|---|---|---|
| ATOM | 41 | CB | AILE A | 3 | −3.415 | −0.444 | 5.054 | 0.50 | 11.62 | C |

| | | | | | | | | | | | |
|---|---|---|---|---|---|---|---|---|---|---|---|
| ANISOU | 41 | CB | AILE A | 3 | 1599 | 1306 | 1508 | 13 | 44 | −83 | C |

| | | | | | | | | | | |
|---|---|---|---|---|---|---|---|---|---|---|
| ATOM | 42 | CB | BILE A | 3 | −3.388 | −0.379 | 5.068 | 0.50 | 11.92 | C |

| | | | | | | | | | | | |
|---|---|---|---|---|---|---|---|---|---|---|---|
| ANISOU | 42 | CB | BILE A | 3 | 1646 | 1355 | 1526 | 22 | −39 | −69 | C |

| | | | | | | | | | | |
|---|---|---|---|---|---|---|---|---|---|---|
| ATOM | 45 | CG1 | AILE A | 3 | −3.961 | −1.067 | 3.767 | 0.50 | 11.53 | C |

| | | | | | | | | | | | |
|---|---|---|---|---|---|---|---|---|---|---|---|
| ANISOU | 45 | CG1 | AILE A | 3 | 1628 | 1186 | 1566 | −31 | −57 | −66 | C |

| | | | | | | | | | | |
|---|---|---|---|---|---|---|---|---|---|---|
| ATOM | 46 | CG1 | BILE A | 3 | −4.076 | −0.592 | 3.717 | 0.50 | 12.37 | C |

| | | | | | | | | | | | |
|---|---|---|---|---|---|---|---|---|---|---|---|
| ANISOU | 46 | CG1 | BILE A | 3 | 1741 | 1392 | 1566 | 5 | 49 | −25 | C |

| | | | | | | | | | | |
|---|---|---|---|---|---|---|---|---|---|---|
| ATOM | 51 | CD1 | AILE A | 3 | −3.116 | −0.790 | 2.547 | 0.50 | 12.15 | C |

| | | | | | | | | | | | |
|---|---|---|---|---|---|---|---|---|---|---|---|
| ANISOU | 51 | CD1 | AILE A | 3 | 1716 | 1311 | 1586 | −113 | −22 | −140 | C |

| | | | | | | | | | | |
|---|---|---|---|---|---|---|---|---|---|---|
| ATOM | 52 | CD1 | BILE A | 3 | −4.519 | −2.010 | 3.478 | 0.50 | 13.71 | C |

| | | | | | | | | | | | |
|---|---|---|---|---|---|---|---|---|---|---|---|
| ANISOU | 52 | CD1 | BILE A | 3 | 2049 | 1529 | 1631 | −58 | −127 | 37 | C |

| | | | | | | | | | | |
|---|---|---|---|---|---|---|---|---|---|---|
| ATOM | 59 | CG2 | AILE A | 3 | −1.990 | −0.916 | 5.296 | 0.50 | 12.37 | C |

| | | | | | | | | | | | |
|---|---|---|---|---|---|---|---|---|---|---|---|
| ANISOU | 59 | CG2 | AILE A | 3 | 1646 | 1514 | 1537 | −14 | −104 | −104 | C |

| | | | | | | | | | | |
|---|---|---|---|---|---|---|---|---|---|---|
| ATOM | 60 | CG2 | BILE A | 3 | −2.080 | −1.157 | 5.120 | 0.50 | 12.61 | C |

| | | | | | | | | | | | |
|---|---|---|---|---|---|---|---|---|---|---|---|
| ANISOU | 60 | CG2 | BILE A | 3 | 1696 | 1494 | 1601 | 4 | −47 | −82 | C |

| | | | | | | | | | | |
|---|---|---|---|---|---|---|---|---|---|---|
| ATOM | 67 | C | ILE A | 3 | −3.771 | −0.178 | 7.523 | 1.00 | 11.71 | C |

| | | | | | | | | | | | |
|---|---|---|---|---|---|---|---|---|---|---|---|
| ANISOU | 67 | C | ILE A | 3 | 1587 | 1358 | 1502 | 10 | −72 | −67 | C |

| | | | | | | | | | | |
|---|---|---|---|---|---|---|---|---|---|---|
| ATOM | 68 | O | ILE A | 3 | −3.743 | 1.039 | 7.649 | 1.00 | 12.74 | O |

| | | | | | | | | | | | |
|---|---|---|---|---|---|---|---|---|---|---|---|
| ANISOU | 68 | O | ILE A | 3 | 1758 | 1511 | 1570 | 30 | −167 | −222 | O |

| | | | | | | | | | | |
|---|---|---|---|---|---|---|---|---|---|---|
| ATOM | 70 | N | VAL A | 4 | −3.358 | −1.009 | 8.473 | 1.00 | 12.47 | N |

| | | | | | | | | | | | |
|---|---|---|---|---|---|---|---|---|---|---|---|
| ANISOU | 70 | N | VAL A | 4 | 1688 | 1450 | 1600 | 29 | −97 | −62 | N |

| | | | | | | | | | | |
|---|---|---|---|---|---|---|---|---|---|---|
| ATOM | 71 | CA | VAL A | 4 | −2.846 | −0.517 | 9.747 | 1.00 | 12.73 | C |

| | | | | | | | | | | | |
|---|---|---|---|---|---|---|---|---|---|---|---|
| ANISOU | 71 | CA | VAL A | 4 | 1710 | 1549 | 1578 | 86 | −100 | −62 | C |

| | | | | | | | | | | |
|---|---|---|---|---|---|---|---|---|---|---|
| ATOM | 73 | CB | VAL A | 4 | −3.722 | −0.982 | 10.925 | 1.00 | 13.31 | C |

| | | | | | | | | | | | |
|---|---|---|---|---|---|---|---|---|---|---|---|
| ANISOU | 73 | CB | VAL A | 4 | 1752 | 1643 | 1659 | 107 | −63 | −108 | C |

| | | | | | | | | | | |
|---|---|---|---|---|---|---|---|---|---|---|
| ATOM | 75 | CG1 | VAL A | 4 | −3.236 | −0.371 | 12.233 | 1.00 | 15.33 | C |

| | | | | | | | | | | | |
|---|---|---|---|---|---|---|---|---|---|---|---|
| ANISOU | 75 | CG1 | VAL A | 4 | 1856 | 2158 | 1811 | 40 | −101 | −201 | C |

| | | | | | | | | | | |
|---|---|---|---|---|---|---|---|---|---|---|
| ATOM | 79 | CG2 | VAL A | 4 | −5.179 | −0.622 | 10.676 | 1.00 | 14.67 | C |

| | | | | | | | | | | | |
|---|---|---|---|---|---|---|---|---|---|---|---|
| ANISOU | 79 | CG2 | VAL A | 4 | 1803 | 2126 | 1646 | 229 | −116 | 24 | C |

| | | | | | | | | | | |
|---|---|---|---|---|---|---|---|---|---|---|
| ATOM | 83 | C | VAL A | 4 | −1.417 | −1.005 | 9.946 | 1.00 | 12.69 | C |

| | | | | | | | | | | | |
|---|---|---|---|---|---|---|---|---|---|---|---|
| ANISOU | 83 | C | VAL A | 4 | 1699 | 1539 | 1582 | 97 | −48 | −56 | C |

| | | | | | | | | | | |
|---|---|---|---|---|---|---|---|---|---|---|
| ATOM | 84 | O | VAL A | 4 | −1.155 | −2.200 | 9.890 | 1.00 | 13.83 | O |

| | | | | | | | | | | | |
|---|---|---|---|---|---|---|---|---|---|---|---|
| ANISOU | 84 | O | VAL A | 4 | 1886 | 1587 | 1781 | 99 | −118 | −68 | O |

| | | | | | | | | | | |
|---|---|---|---|---|---|---|---|---|---|---|
| ATOM | 86 | N | TYR A | 5 | −0.506 | −0.061 | 10.162 | 1.00 | 13.62 | N |

| | | | | | | | | | | | |
|---|---|---|---|---|---|---|---|---|---|---|---|
| ANISOU | 86 | N | TYR A | 5 | 1821 | 1619 | 1733 | 95 | −41 | −90 | N |

| | | | | | | | | | | |
|---|---|---|---|---|---|---|---|---|---|---|
| ATOM | 87 | CA | TYR A | 5 | 0.899 | −0.350 | 10.421 | 1.00 | 14.31 | C |

| | | | | | | | | | | | |
|---|---|---|---|---|---|---|---|---|---|---|---|
| ANISOU | 87 | CA | TYR A | 5 | 1860 | 1733 | 1841 | 69 | −16 | −103 | C |

| | | | | | | | | | | |
|---|---|---|---|---|---|---|---|---|---|---|
| ATOM | 89 | CB | TYR A | 5 | 1.787 | 0.535 | 9.551 | 1.00 | 14.99 | C |

| | | | | | | | | | | | |
|---|---|---|---|---|---|---|---|---|---|---|---|
| ANISOU | 89 | CB | TYR A | 5 | 1911 | 1833 | 1949 | 24 | 32 | −154 | C |

| | | | | | | | | | | |
|---|---|---|---|---|---|---|---|---|---|---|
| ATOM | 92 | CG | TYR A | 5 | 1.670 | 0.308 | 8.061 | 1.00 | 15.72 | C |

| | | | | | | | | | | | |
|---|---|---|---|---|---|---|---|---|---|---|---|
| ANISOU | 92 | CG | TYR A | 5 | 1967 | 1953 | 2053 | −51 | 33 | −145 | C |

| | | | | | | | | | | |
|---|---|---|---|---|---|---|---|---|---|---|
| ATOM | 93 | CD1 | TYR A | 5 | 0.834 | 1.093 | 7.275 | 1.00 | 15.75 | C |

| | | | | | | | | | | | |
|---|---|---|---|---|---|---|---|---|---|---|---|
| ANISOU | 93 | CD1 | TYR A | 5 | 1766 | 2056 | 2161 | −94 | −132 | −183 | C |

| | | | | | | | | | | |
|---|---|---|---|---|---|---|---|---|---|---|
| ATOM | 95 | CE1 | TYR A | 5 | 0.745 | 0.895 | 5.903 | 1.00 | 16.43 | C |

| | | | | | | | | | | | |
|---|---|---|---|---|---|---|---|---|---|---|---|
| ANISOU | 95 | CE1 | TYR A | 5 | 1873 | 2158 | 2211 | −124 | −42 | −213 | C |

| | | | | | | | | | | |
|---|---|---|---|---|---|---|---|---|---|---|
| ATOM | 97 | CZ | TYR A | 5 | 1.501 | −0.100 | 5.302 | 1.00 | 16.92 | C |

| | | | | | | | | | | | |
|---|---|---|---|---|---|---|---|---|---|---|---|
| ANISOU | 97 | CZ | TYR A | 5 | 1817 | 2332 | 2277 | −119 | −80 | −115 | C |

| | | | | | | | | | | |
|---|---|---|---|---|---|---|---|---|---|---|
| ATOM | 98 | OH | TYR A | 5 | 1.429 | −0.320 | 3.945 | 1.00 | 18.99 | O |

| | | | | | | | | | | | |
|---|---|---|---|---|---|---|---|---|---|---|---|
| ANISOU | 98 | OH | TYR A | 5 | 1975 | 2901 | 2338 | −400 | 206 | −177 | O |

| | | | | | | | | | | |
|---|---|---|---|---|---|---|---|---|---|---|
| ATOM | 100 | CE2 | TYR A | 5 | 2.340 | −0.884 | 6.063 | 1.00 | 16.98 | C |

| | | | | | | | | | | | |
|---|---|---|---|---|---|---|---|---|---|---|---|
| ANISOU | 100 | CE2 | TYR A | 5 | 1845 | 2291 | 2314 | −72 | 12 | −222 | C |

| | | | | | | | | | | |
|---|---|---|---|---|---|---|---|---|---|---|
| ATOM | 102 | CD2 | TYR A | 5 | 2.423 | −0.676 | 7.434 | 1.00 | 16.52 | C |

| | | | | | | | | | | | |
|---|---|---|---|---|---|---|---|---|---|---|---|
| ANISOU | 102 | CD2 | TYR A | 5 | 1938 | 2108 | 2229 | −10 | 40 | −190 | C |

| | | | | | | | | | | |
|---|---|---|---|---|---|---|---|---|---|---|
| ATOM | 104 | C | TYR A | 5 | 1.225 | −0.079 | 11.885 | 1.00 | 15.13 | C |

| | | | | | | | | | | | |
|---|---|---|---|---|---|---|---|---|---|---|---|
| ANISOU | 104 | C | TYR A | 5 | 1953 | 1870 | 1924 | 86 | −67 | −143 | C |

| | | | | | | | | | | |
|---|---|---|---|---|---|---|---|---|---|---|
| ATOM | 105 | O | TYR A | 5 | 1.109 | 1.052 | 12.344 | 1.00 | 17.42 | O |

| | | | | | | | | | | | |
|---|---|---|---|---|---|---|---|---|---|---|---|
| ANISOU | 105 | O | TYR A | 5 | 2310 | 2155 | 2150 | 75 | −116 | −176 | O |

| | | | | | | | | | | |
|---|---|---|---|---|---|---|---|---|---|---|
| ATOM | 107 | N | LYS A | 6 | 1.636 | −1.116 | 12.609 | 1.00 | 16.68 | N |

| | | | | | | | | | | | |
|---|---|---|---|---|---|---|---|---|---|---|---|
| ANISOU | 107 | N | LYS A | 6 | 2112 | 2180 | 2047 | 80 | −84 | −118 | N |

| | | | | | | | | | | |
|---|---|---|---|---|---|---|---|---|---|---|
| ATOM | 108 | CA | LYS A | 6 | 2.030 | −0.993 | 14.013 | 1.00 | 17.50 | C |

| | | | | | | | | | | | |
|---|---|---|---|---|---|---|---|---|---|---|---|
| ANISOU | 108 | CA | LYS A | 6 | 2151 | 2390 | 2106 | 113 | −119 | −104 | C |

| | | | | | | | | | | |
|---|---|---|---|---|---|---|---|---|---|---|
| ATOM | 110 | CB | LYS A | 6 | 1.019 | −1.681 | 14.926 | 1.00 | 17.80 | C |

| | | | | | | | | | | | |
|---|---|---|---|---|---|---|---|---|---|---|---|
| ANISOU | 110 | CB | LYS A | 6 | 2212 | 2457 | 2093 | 147 | −93 | −122 | C |

| | | | | | | | | | | |
|---|---|---|---|---|---|---|---|---|---|---|
| ATOM | 113 | CG | LYS A | 6 | −0.426 | −1.274 | 14.731 | 1.00 | 18.84 | C |

| | | | | | | | | | | | |
|---|---|---|---|---|---|---|---|---|---|---|---|
| ANISOU | 113 | CG | LYS A | 6 | 2344 | 2587 | 2225 | 225 | −178 | 32 | C |

| | | | | | | | | | | |
|---|---|---|---|---|---|---|---|---|---|---|
| ATOM | 116 | CD | LYS A | 6 | −1.288 | −1.862 | 15.840 | 1.00 | 19.91 | C |

| | | | | | | | | | | | |
|---|---|---|---|---|---|---|---|---|---|---|---|
| ANISOU | 116 | CD | LYS A | 6 | 2469 | 2760 | 2336 | 244 | −80 | 188 | C |

| | | | | | | | | | | |
|---|---|---|---|---|---|---|---|---|---|---|
| ATOM | 119 | CE | LYS A | 6 | −2.756 | −1.546 | 15.661 | 1.00 | 21.19 | C |

| | | | | | | | | | | | |
|---|---|---|---|---|---|---|---|---|---|---|---|
| ANISOU | 119 | CE | LYS A | 6 | 2542 | 3024 | 2486 | 237 | −74 | 363 | C |

| | | | | | | | | | | |
|---|---|---|---|---|---|---|---|---|---|---|
| ATOM | 122 | NZ | LYS A | 6 | −3.507 | −1.738 | 16.934 | 1.00 | 23.23 | N |

| | | | | | | | | | | | |
|---|---|---|---|---|---|---|---|---|---|---|---|
| ANISOU | 122 | NZ | LYS A | 6 | 2811 | 3406 | 2610 | 295 | 70 | 552 | N |

| | | | | | | | | | | |
|---|---|---|---|---|---|---|---|---|---|---|
| ATOM | 126 | C | LYS A | 6 | 3.389 | −1.638 | 14.246 | 1.00 | 18.75 | C |

| | | | | | | | | | | | |
|---|---|---|---|---|---|---|---|---|---|---|---|
| ANISOU | 126 | C | LYS A | 6 | 2254 | 2640 | 2227 | 125 | −124 | −141 | C |

| | | | | | | | | | | |
|---|---|---|---|---|---|---|---|---|---|---|
| ATOM | 127 | O | LYS A | 6 | 3.909 | −2.369 | 13.402 | 1.00 | 20.39 | O |

| | | | | | | | | | | | |
|---|---|---|---|---|---|---|---|---|---|---|---|
| ANISOU | 127 | O | LYS A | 6 | 2422 | 2890 | 2431 | 155 | 144 | −96 | O |

| | | | | | | | | | | |
|---|---|---|---|---|---|---|---|---|---|---|
| ATOM | 129 | OT | LYS A | 6 | 3.987 | −1.462 | 15.308 | 1.00 | 21.04 | O |

| | | | | | | | | | | | |
|---|---|---|---|---|---|---|---|---|---|---|---|
| ANISOU | 129 | OT | LYS A | 6 | 2500 | 2984 | 2509 | 118 | −135 | −101 | O |

| | | | | | | | | | | |
|---|---|---|---|---|---|---|---|---|---|---|
| ATOM | 130 | O | HOH B | 1 | 6.439 | −4.591 | 13.410 | 1.00 | 27.61 | O |

| | | | | | | | | | | | |
|---|---|---|---|---|---|---|---|---|---|---|---|
| ANISOU | 130 | O | HOH B | 1 | 4665 | 2686 | 3139 | 80 | 2094 | 125 | O |

| | | | | | | | | | | |
|---|---|---|---|---|---|---|---|---|---|---|
| ATOM | 133 | O | HOH B | 2 | 8.266 | −0.903 | 15.154 | 1.00 | 43.30 | O |

| | | | | | | | | | | | |
|---|---|---|---|---|---|---|---|---|---|---|---|
| ANISOU | 133 | O | HOH B | 2 | 3380 | 7635 | 5436 | −2653 | 2948 | −4381 | O |

TABLE 6
Atomic coordinates of structure of an amyloid-forming peptide
VQIVYK (SEQ ID NO: 2) from the tau protein in complex with DDNP

HEADER PROTEIN FIBRIL
TITLE STRUCTURE OF AN AMYLOID FORMING PEPTIDE VQIVYK FROM THE TAU PROTEIN IN
TITLE 2 COMPLEX WITH DDNP
AUTHOR M. LANDAU, D. EISENBERG

| REMARK | 3 | |
|---|---|---|
| REMARK | 3 | REFINEMENT. |

| | | | |
|---|---|---|---|
| REMARK | 3 | PROGRAM | : REFMAC 5.5.0109 |
| REMARK | 3 | AUTHORS | : MURSHUDOV, VAGIN, DODSON |

| REMARK | 3 | |
|---|---|---|
| REMARK | 3 | REFINEMENT TARGET: MAXIMUM LIKELIHOOD |

| REMARK | 3 | |
|---|---|---|
| REMARK | 3 | DATA USED IN REFINEMENT. |

| | | | |
|---|---|---|---|
| REMARK | 3 | RESOLUTION RANGE HIGH (ANGSTROMS) | : 1.20 |
| REMARK | 3 | RESOLUTION RANGE LOW (ANGSTROMS) | : 33.20 |
| REMARK | 3 | DATA CUTOFF (SIGMA(F)) | : NONE |
| REMARK | 3 | COMPLETENESS FOR RANGE (%) | : 82.94 |
| REMARK | 3 | NUMBER OF REFLECTIONS | : 1201 |

| REMARK | 3 | |
|---|---|---|
| REMARK | 3 | FIT TO DATA USED IN REFINEMENT. |

| | | | |
|---|---|---|---|
| REMARK | 3 | CROSS-VALIDATION METHOD | : THROUGHOUT |
| REMARK | 3 | FREE R VALUE TEST SET SELECTION | : RANDOM |
| REMARK | 3 | R VALUE (WORKING + TEST SET) | : 0.15994 |
| REMARK | 3 | R VALUE (WORKING SET) | : 0.15828 |
| REMARK | 3 | FREE R VALUE | : 0.17446 |
| REMARK | 3 | FREE R VALUE TEST SET SIZE (%) | : 9.5 |
| REMARK | 3 | FREE R VALUE TEST SET COUNT | : 126 |

| REMARK | 3 | |
|---|---|---|
| REMARK | 3 | FIT IN THE HIGHEST RESOLUTION BIN. |

| | | | |
|---|---|---|---|
| REMARK | 3 | TOTAL NUMBER OF BINS USED | : 5 |
| REMARK | 3 | BIN RESOLUTION RANGE HIGH | : 1.204 |
| REMARK | 3 | BIN RESOLUTION RANGE LOW | : 1.346 |
| REMARK | 3 | REFLECTION IN BIN (WORKING SET) | : 278 |
| REMARK | 3 | BIN COMPLETENESS (WORKING + TEST) (%) | : 74.15 |
| REMARK | 3 | BIN R VALUE (WORKING SET) | : 0.202 |
| REMARK | 3 | BIN FREE R VALUE SET COUNT | : 26 |
| REMARK | 3 | BIN FREE R VALUE | : 0.235 |

| REMARK | 3 | |
|---|---|---|
| REMARK | 3 | NUMBER OF NON-HYDROGEN ATOMS USED IN REFINEMENT. |

| | | | |
|---|---|---|---|
| REMARK | 3 | ALL ATOMS | : 61 |

| REMARK | 3 | |
|---|---|---|
| REMARK | 3 | B VALUES. |

| | | | |
|---|---|---|---|
| REMARK | 3 | FROM WILSON PLOT (A**2) | : NULL |
| REMARK | 3 | MEAN B VALUE (OVERALL, A**2) | : 3.685 |

| | | |
|---|---|---|
| REMARK | 3 | OVERALL ANISOTROPIC B VALUE. |

| | | | |
|---|---|---|---|
| REMARK | 3 | B11 (A**2): | −0.10 |
| REMARK | 3 | B22 (A**2): | 0.11 |
| REMARK | 3 | B33 (A**2): | −0.05 |
| REMARK | 3 | B12 (A**2): | 0.00 |
| REMARK | 3 | B13 (A**2): | −0.06 |
| REMARK | 3 | B23 (A**2): | 0.00 |

| REMARK | 3 | |
|---|---|---|
| REMARK | 3 | ESTIMATED OVERALL COORDINATE ERROR. |

| | | | |
|---|---|---|---|
| REMARK | 3 | ESU BASED ON R VALUE (A) | : 0.064 |
| REMARK | 3 | ESU BASED ON FREE R VALUE (A) | : 0.052 |
| REMARK | 3 | ESU BASED ON MAXIMUM LIKELIHOOD (A) | : NULL |
| REMARK | 3 | ESU FOR B VALUES BASED ON MAXIMUM LIKELIHOOD (A**2) | : NULL |

| REMARK | 3 | |
|---|---|---|
| REMARK | 3 | CORRELATION COEFFICIENTS. |

| | | | |
|---|---|---|---|
| REMARK | 3 | CORRELATION COEFFICIENT FO-FC | : 0.959 |
| REMARK | 3 | CORRELATION COEFFICIENT FO-FC FREE | : 0.937 |

| REMARK | 3 | | | | |
|---|---|---|---|---|---|
| REMARK | 3 | RMS DEVIATIONS FROM IDEAL VALUES | COUNT | RMS | WEIGHT |

| | | | | | |
|---|---|---|---|---|---|
| REMARK | 3 | BOND LENGTHS REFINED ATOMS (A): | 59 ; | 0.011 ; | 0.024 |
| REMARK | 3 | BOND LENGTHS OTHERS (A): | 38; | 0.000 ; | 0.020 |
| REMARK | 3 | BOND ANGLES REFINED ATOMS (DEGREES): | 81; | 1.271 ; | 2.014 |
| REMARK | 3 | BOND ANGLES OTHERS (DEGREES): | 97; | 3.577 ; | 3.000 |
| REMARK | 3 | TORSION ANGLES, PERIOD 1 (DEGRESS): | 7; | 4.469 ; | 5.000 |
| REMARK | 3 | TORSION ANGLES, PERIOD 2 (DEGREES): | 2; | 42.283 ; | 25.000 |
| REMARK | 3 | TORSION ANGLES, PERIOD 3 (DEGREES): | 13; | 7.079 ; | 15.000 |
| REMARK | 3 | CHIRAL-CENTER RESTRAINTS (A**3): | 11; | 0.102 ; | 0.200 |
| REMARK | 3 | GENERAL PLANES REFINED ATOMS (A): | 58; | 0.004 ; | 0.020 |
| REMARK | 3 | GENERAL PLANES OTHERS (A): | 10; | 0.001 ; | 0.020 |

| | | | | | |
|---|---|---|---|---|---|
| REMARK | 3 | | | | |
| REMARK | 3 | ISOTROPIC THERMAL FACTOR RESTRAINTS. | COUNT | RMS | WEIGHT |

| | | | | | |
|---|---|---|---|---|---|
| REMARK | 3 | MAIN-CHAIN BOND REFINED ATOMS (A**2): | 33; | 1.835 ; | 1.500 |
| REMARK | 3 | MAIN-CHAIN BOND OTHER ATOMS (A**2): | 12; | 1.099 ; | 1.500 |
| REMARK | 3 | MAIN-CHAIN ANGLE REFINED ATOMS (A**2): | 57; | 2.230 ; | 2.000 |
| REMARK | 3 | SIDE-CHAIN BOND REFINED ATOMS (A**2): | 26; | 2.206 ; | 3.000 |
| REMARK | 3 | SIDE-CHAIN ANGLE REFINED ATOMS (A**2): | 23; | 2.403 ; | 4.500 |

| | | | | | |
|---|---|---|---|---|---|
| REMARK | 3 | | | | |
| REMARK | 3 | ANISOTROPIC THERMAL FACTOR RESTRAINTS. | COUNT | RMS | WEIGHT |

| | | | | | |
|---|---|---|---|---|---|
| REMARK | 3 | RIGID-BOND RESTRAINTS (A**2): | 97; | 1.449 ; | 3.000 |

| REMARK | 3 | |
|---|---|---|
| REMARK | 3 | NCS RESTRAINTS STATISTICS |

| | | |
|---|---|---|
| REMARK | 3 | NUMBER OF NCS GROUPS: NULL |

| | | |
|---|---|---|
| REMARK | 3 | |
| REMARK | 3 | TWIN DETAILS |

| | | |
|---|---|---|
| REMARK | 3 | NUMBER OF TWIN DOMAINS: NULL |

| | | |
|---|---|---|
| REMARK | 3 | |
| REMARK | 3 | |
| REMARK | 3 | TLS DETAILS |

| | | |
|---|---|---|
| REMARK | 3 | NUMBER OF TLS GROUPS: NULL |

| REMARK | 3 | |
|---|---|---|
| REMARK | 3 | |
| REMARK | 3 | BULK SOLVENT MODELLING. |

| REMARK | 3 | METHOD USED: NONE | |
|---|---|---|---|
| REMARK | 3 | PARAMETERS FOR MASK CACLULATION | |
| REMARK | 3 | VDW PROBE RADIOS | : NULL |
| REMARK | 3 | ION PROBE RADIUS | : NULL |
| REMARK | 3 | SHRINKAGE RADIUS | : NULL |

| REMARK | 3 | | | | | | |
|---|---|---|---|---|---|---|---|
| REMARK | 3 | OTHER REFINEMENT REMARKS: | | | | | |
| REMARK | 3 | HYDROGENS HAVE BEEN ADDED IN THE RIDING POSITIONS | | | | | |
| REMARK | 3 | U VALUES: REFINED INDIVIDUALLY | | | | | |
| REMARK | 3 | | | | | | |
| CRYST1 | 28.156 | 4.856 | 35.269 | 90.00 | 109.70 | 90.00 | C 1 2 1 |

| | | | | |
|---|---|---|---|---|
| SCALE1 | 0.035516 | 0.000000 | 0.012718 | 0.00000 |
| SCALE2 | 0.000000 | 0.205931 | 0.000000 | 0.00000 |
| SCALE3 | 0.000000 | 0.000000 | 0.030117 | 0.00000 |

| | | | | | | | | | | |
|---|---|---|---|---|---|---|---|---|---|---|
| ATOM | 1 | N | VAL A | 1 | −11.184 | 0.240 | 2.439 | 1.00 | 5.48 | N |

| | | | | | | | | | | | |
|---|---|---|---|---|---|---|---|---|---|---|---|
| ANISOU | 1 | N | VAL A | 1 | 575 | 776 | 728 | 137 | −131 | 120 | N |

| | | | | | | | | | | |
|---|---|---|---|---|---|---|---|---|---|---|
| ATOM | 2 | CA | VAL A | 1 | −9.826 | −0.369 | 2.574 | 1.00 | 3.34 | C |

| | | | | | | | | | | | |
|---|---|---|---|---|---|---|---|---|---|---|---|
| ANISOU | 2 | CA | VAL A | 1 | 380 | 392 | 496 | 74 | −124 | −63 | C |

| | | | | | | | | | | |
|---|---|---|---|---|---|---|---|---|---|---|
| ATOM | 4 | CB | VAL A | 1 | −8.984 | −0.177 | 1.284 | 1.00 | 4.49 | C |

| | | | | | | | | | | | |
|---|---|---|---|---|---|---|---|---|---|---|---|
| ANISOU | 4 | CB | VAL A | 1 | 728 | 584 | 392 | 76 | −71 | 43 | C |

| | | | | | | | | | | |
|---|---|---|---|---|---|---|---|---|---|---|
| ATOM | 6 | CG1 | VAL A | 1 | −7.571 | −0.686 | 1.486 | 1.00 | 5.92 | C |

| | | | | | | | | | | | |
|---|---|---|---|---|---|---|---|---|---|---|---|
| ANISOU | 6 | CG1 | VAL A | 1 | 529 | 1061 | 657 | 46 | −52 | −183 | C |

| | | | | | | | | | | |
|---|---|---|---|---|---|---|---|---|---|---|
| ATOM | 10 | CG2 | VAL A | 1 | −9.650 | −0.914 | 0.109 | 1.00 | 5.90 | C |

| | | | | | | | | | | | |
|---|---|---|---|---|---|---|---|---|---|---|---|
| ANISOU | 10 | CG2 | VAL A | 1 | 954 | 848 | 438 | 35 | −287 | −186 | C |

| | | | | | | | | | | |
|---|---|---|---|---|---|---|---|---|---|---|
| ATOM | 14 | C | VAL A | 1 | −9.133 | 0.290 | 3.749 | 1.00 | 4.00 | C |

| | | | | | | | | | | | |
|---|---|---|---|---|---|---|---|---|---|---|---|
| ANISOU | 14 | C | VAL A | 1 | 441 | 467 | 608 | −109 | 10 | −137 | C |

| | | | | | | | | | | |
|---|---|---|---|---|---|---|---|---|---|---|
| ATOM | 15 | O | VAL A | 1 | −9.011 | 1.514 | 3.796 | 1.00 | 3.30 | O |

| | | | | | | | | | | | |
|---|---|---|---|---|---|---|---|---|---|---|---|
| ANISOU | 15 | O | VAL A | 1 | 437 | 363 | 451 | 68 | −82 | 87 | O |

| | | | | | | | | | | |
|---|---|---|---|---|---|---|---|---|---|---|
| ATOM | 19 | N | GLN A | 2 | −8.654 | −0.528 | 4.687 | 1.00 | 3.35 | N |

| | | | | | | | | | | | |
|---|---|---|---|---|---|---|---|---|---|---|---|
| ANISOU | 19 | N | GLN A | 2 | 423 | 307 | 541 | −95 | −219 | 124 | N |

| | | | | | | | | | | |
|---|---|---|---|---|---|---|---|---|---|---|
| ATOM | 20 | CA | GLN A | 2 | −7.898 | −0.014 | 5.831 | 1.00 | 3.13 | C |

| | | | | | | | | | | | |
|---|---|---|---|---|---|---|---|---|---|---|---|
| ANISOU | 20 | CA | GLN A | 2 | 331 | 339 | 520 | 76 | −27 | 20 | C |

| | | | | | | | | | | |
|---|---|---|---|---|---|---|---|---|---|---|
| ATOM | 22 | CB | GLN A | 2 | −8.621 | −0.361 | 7.133 | 1.00 | 2.73 | C |

| | | | | | | | | | | | |
|---|---|---|---|---|---|---|---|---|---|---|---|
| ANISOU | 22 | CB | GLN A | 2 | 327 | 310 | 398 | 49 | −81 | −91 | C |

| | | | | | | | | | | |
|---|---|---|---|---|---|---|---|---|---|---|
| ATOM | 25 | CG | GLN A | 2 | −7.988 | 0.314 | 8.362 | 1.00 | 3.60 | C |

| | | | | | | | | | | | |
|---|---|---|---|---|---|---|---|---|---|---|---|
| ANISOU | 25 | CG | GLN A | 2 | 271 | 608 | 487 | 73 | 5 | −79 | C |

| | | | | | | | | | | |
|---|---|---|---|---|---|---|---|---|---|---|
| ATOM | 28 | CD | GLN A | 2 | −8.594 | −0.130 | 9.673 | 1.00 | 3.29 | C |

| | | | | | | | | | | | |
|---|---|---|---|---|---|---|---|---|---|---|---|
| ANISOU | 28 | CD | GLN A | 2 | 348 | 512 | 388 | 6 | −82 | 120 | C |

| | | | | | | | | | | |
|---|---|---|---|---|---|---|---|---|---|---|
| ATOM | 29 | OE1 | GLN A | 2 | −8.557 | −1.314 | 10.012 | 1.00 | 4.14 | O |

| | | | | | | | | | | | |
|---|---|---|---|---|---|---|---|---|---|---|---|
| ANISOU | 29 | OE1 | GLN A | 2 | 472 | 436 | 666 | −107 | 164 | −27 | O |

| | | | | | | | | | | |
|---|---|---|---|---|---|---|---|---|---|---|
| ATOM | 30 | NE2 | GLN A | 2 | −9.144 | 0.809 | 10.425 | 1.00 | 3.40 | N |

| | | | | | | | | | | | |
|---|---|---|---|---|---|---|---|---|---|---|---|
| ANISOU | 30 | NE2 | GLN A | 2 | 381 | 395 | 515 | 54 | 174 | −19 | N |

| | | | | | | | | | | |
|---|---|---|---|---|---|---|---|---|---|---|
| ATOM | 33 | C | GLN A | 2 | −6.517 | −0.659 | 5.871 | 1.00 | 2.94 | C |

| | | | | | | | | | | | |
|---|---|---|---|---|---|---|---|---|---|---|---|
| ANISOU | 33 | C | GLN A | 2 | 393 | 371 | 351 | 116 | 43 | 1 | C |

| | | | | | | | | | | |
|---|---|---|---|---|---|---|---|---|---|---|
| ATOM | 34 | O | GLN A | 2 | −6.402 | −1.890 | 5.854 | 1.00 | 2.77 | O |

| | | | | | | | | | | | |
|---|---|---|---|---|---|---|---|---|---|---|---|
| ANISOU | 34 | O | GLN A | 2 | 282 | 258 | 513 | 11 | 36 | 10 | O |

| | | | | | | | | | | |
|---|---|---|---|---|---|---|---|---|---|---|
| ATOM | 36 | N | ILE A | 3 | −5.476 | 0.174 | 5.945 | 1.00 | 2.21 | N |

| | | | | | | | | | | | |
|---|---|---|---|---|---|---|---|---|---|---|---|
| ANISOU | 36 | N | ILE A | 3 | 318 | 263 | 257 | 25 | −15 | −5 | N |

| | | | | | | | | | | |
|---|---|---|---|---|---|---|---|---|---|---|
| ATOM | 37 | CA | AILE A | 3 | −4.117 | −0.329 | 6.170 | 0.40 | 2.65 | C |

| | | | | | | | | | | | |
|---|---|---|---|---|---|---|---|---|---|---|---|
| ANISOU | 37 | CA | AILE A | 3 | 320 | 335 | 350 | 73 | −80 | −87 | C |

| | | | | | | | | | | |
|---|---|---|---|---|---|---|---|---|---|---|
| ATOM | 38 | CA | BILE A | 3 | −4.110 | −0.317 | 6.151 | 0.60 | 2.79 | C |

| | | | | | | | | | | | |
|---|---|---|---|---|---|---|---|---|---|---|---|
| ANISOU | 38 | CA | BILE A | 3 | 331 | 359 | 369 | 88 | −93 | −109 | C |

| | | | | | | | | | | |
|---|---|---|---|---|---|---|---|---|---|---|
| ATOM | 41 | CB | AILE A | 3 | −3.170 | −0.043 | 4.994 | 0.40 | 3.82 | C |

| | | | | | | | | | | | |
|---|---|---|---|---|---|---|---|---|---|---|---|
| ANISOU | 41 | CB | AILE A | 3 | 455 | 516 | 478 | 35 | −82 | −43 | C |

| | | | | | | | | | | |
|---|---|---|---|---|---|---|---|---|---|---|
| ATOM | 42 | CB | BILE A | 3 | −3.167 | 0.052 | 4.980 | 0.60 | 4.48 | C |

| | | | | | | | | | | | |
|---|---|---|---|---|---|---|---|---|---|---|---|
| ANISOU | 42 | CB | BILE A | 3 | 533 | 653 | 517 | 17 | −91 | −71 | C |

| | | | | | | | | | | |
|---|---|---|---|---|---|---|---|---|---|---|
| ATOM | 45 | CG1 | AILE A | 3 | −3.722 | −0.681 | 3.710 | 0.40 | 4.44 | C |

| | | | | | | | | | | | |
|---|---|---|---|---|---|---|---|---|---|---|---|
| ANISOU | 45 | CG1 | AILE A | 3 | 538 | 511 | 639 | 68 | −3 | −63 | C |

| | | | | | | | | | | |
|---|---|---|---|---|---|---|---|---|---|---|
| ATOM | 46 | CG1 | BILE A | 3 | −3.849 | −0.185 | 3.616 | 0.60 | 7.12 | C |

| | | | | | | | | | | | |
|---|---|---|---|---|---|---|---|---|---|---|---|
| ANISOU | 46 | CG1 | BILE A | 3 | 830 | 1163 | 710 | 98 | −109 | −186 | C |

| | | | | | | | | | | |
|---|---|---|---|---|---|---|---|---|---|---|
| ATOM | 51 | CD1 | AILE A | 3 | −2.819 | −0.483 | 2.495 | 0.40 | 3.72 | C |

| | | | | | | | | | | | |
|---|---|---|---|---|---|---|---|---|---|---|---|
| ANISOU | 51 | CD1 | AILE A | 3 | 369 | 477 | 566 | −153 | −12 | 93 | C |

| | | | | | | | | | | |
|---|---|---|---|---|---|---|---|---|---|---|
| ATOM | 52 | CD1 | BILE A | 3 | −4.270 | −1.567 | 3.347 | 0.60 | 9.51 | C |

| | | | | | | | | | | | |
|---|---|---|---|---|---|---|---|---|---|---|---|
| ANISOU | 52 | CD1 | BILE A | 3 | 1263 | 1185 | 1167 | 260 | −115 | 6 | C |

| | | | | | | | | | | |
|---|---|---|---|---|---|---|---|---|---|---|
| ATOM | 59 | CG2 | AILE A | 3 | −1.772 | −0.584 | 5.297 | 0.40 | 2.88 | C |

| | | | | | | | | | | | |
|---|---|---|---|---|---|---|---|---|---|---|---|
| ANISOU | 59 | CG2 | AILE A | 3 | 320 | 375 | 399 | 3 | 31 | −70 | C |

| | | | | | | | | | | |
|---|---|---|---|---|---|---|---|---|---|---|
| ATOM | 60 | CG2 | BILE A | 3 | −1.861 | −0.729 | 5.091 | 0.60 | 4.36 | C |

| | | | | | | | | | | | |
|---|---|---|---|---|---|---|---|---|---|---|---|
| ANISOU | 60 | CG2 | BILE A | 3 | 393 | 648 | 614 | −85 | −3 | −76 | C |

| | | | | | | | | | | |
|---|---|---|---|---|---|---|---|---|---|---|
| ATOM | 67 | C | ILE A | 3 | −3.582 | 0.348 | 7.421 | 1.00 | 2.85 | C |

| | | | | | | | | | | | |
|---|---|---|---|---|---|---|---|---|---|---|---|
| ANISOU | 67 | C | ILE A | 3 | 390 | 360 | 329 | −115 | 49 | −20 | C |

| | | | | | | | | | | |
|---|---|---|---|---|---|---|---|---|---|---|
| ATOM | 68 | O | ILE A | 3 | −3.562 | 1.578 | 7.501 | 1.00 | 2.57 | O |

| | | | | | | | | | | | |
|---|---|---|---|---|---|---|---|---|---|---|---|
| ANISOU | 68 | O | ILE A | 3 | 441 | 257 | 274 | 17 | −48 | 0 | O |

| | | | | | | | | | | |
|---|---|---|---|---|---|---|---|---|---|---|
| ATOM | 70 | N | VAL A | 4 | −3.163 | −0.463 | 8.397 | 1.00 | 2.71 | N |

| | | | | | | | | | | | |
|---|---|---|---|---|---|---|---|---|---|---|---|
| ANISOU | 70 | N | VAL A | 4 | 396 | 262 | 369 | −35 | −126 | 31 | N |

| | | | | | | | | | | |
|---|---|---|---|---|---|---|---|---|---|---|
| ATOM | 71 | CA | VAL A | 4 | −2.655 | 0.081 | 9.645 | 1.00 | 3.49 | C |

| | | | | | | | | | | | |
|---|---|---|---|---|---|---|---|---|---|---|---|
| ANISOU | 71 | CA | VAL A | 4 | 320 | 534 | 472 | 115 | 48 | 26 | C |

| | | | | | | | | | | |
|---|---|---|---|---|---|---|---|---|---|---|
| ATOM | 73 | CB | VAL A | 4 | −3.563 | −0.304 | 10.837 | 1.00 | 4.30 | C |

| | | | | | | | | | | | |
|---|---|---|---|---|---|---|---|---|---|---|---|
| ANISOU | 73 | CB | VAL A | 4 | 366 | 745 | 524 | 13 | 48 | 20 | C |

| | | | | | | | | | | |
|---|---|---|---|---|---|---|---|---|---|---|
| ATOM | 75 | CG1 | VAL A | 4 | −3.026 | 0.304 | 12.160 | 1.00 | 6.86 | C |

| | | | | | | | | | | | |
|---|---|---|---|---|---|---|---|---|---|---|---|
| ANISOU | 75 | CG1 | VAL A | 4 | 405 | 1526 | 675 | 219 | 93 | −62 | C |

| | | | | | | | | | | |
|---|---|---|---|---|---|---|---|---|---|---|
| ATOM | 79 | CG2 | VAL A | 4 | −5.002 | 0.145 | 10.581 | 1.00 | 6.07 | C |

| | | | | | | | | | | | |
|---|---|---|---|---|---|---|---|---|---|---|---|
| ANISOU | 79 | CG2 | VAL A | 4 | 484 | 1298 | 521 | 239 | 0 | −3 | C |

| | | | | | | | | | | |
|---|---|---|---|---|---|---|---|---|---|---|
| ATOM | 83 | C | VAL A | 4 | −1.239 | −0.464 | 9.863 | 1.00 | 3.45 | C |

| | | | | | | | | | | | |
|---|---|---|---|---|---|---|---|---|---|---|---|
| ANISOU | 83 | C | VAL A | 4 | 445 | 515 | 350 | 74 | −76 | 93 | C |

| | | | | | | | | | | |
|---|---|---|---|---|---|---|---|---|---|---|
| ATOM | 84 | O | VAL A | 4 | −1.019 | −1.683 | 9.822 | 1.00 | 4.00 | O |

| | | | | | | | | | | | |
|---|---|---|---|---|---|---|---|---|---|---|---|
| ANISOU | 84 | O | VAL A | 4 | 417 | 415 | 687 | 60 | −166 | 124 | O |

| | | | | | | | | | | |
|---|---|---|---|---|---|---|---|---|---|---|
| ATOM | 86 | N | TYR A | 5 | −0.299 | 0.457 | 10.107 | 1.00 | 3.02 | N |

| | | | | | | | | | | | |
|---|---|---|---|---|---|---|---|---|---|---|---|
| ANISOU | 86 | N | TYR A | 5 | 455 | 322 | 371 | 115 | −98 | −71 | N |

| | | | | | | | | | | |
|---|---|---|---|---|---|---|---|---|---|---|
| ATOM | 87 | CA | TYR A | 5 | 1.103 | 0.116 | 10.330 | 1.00 | 2.73 | C |

| | | | | | | | | | | | |
|---|---|---|---|---|---|---|---|---|---|---|---|
| ANISOU | 87 | CA | TYR A | 5 | 274 | 318 | 442 | 13 | 3 | −98 | C |

| | | | | | | | | | | |
|---|---|---|---|---|---|---|---|---|---|---|
| ATOM | 89 | CB | TYR A | 5 | 2.004 | 0.988 | 9.468 | 1.00 | 3.64 | C |

| | | | | | | | | | | | |
|---|---|---|---|---|---|---|---|---|---|---|---|
| ANISOU | 89 | CB | TYR A | 5 | 345 | 284 | 751 | 28 | 19 | −98 | C |

| | | | | | | | | | | |
|---|---|---|---|---|---|---|---|---|---|---|
| ATOM | 92 | CG | TYR A | 5 | 1.895 | 0.749 | 7.983 | 1.00 | 3.69 | C |

| | | | | | | | | | | | |
|---|---|---|---|---|---|---|---|---|---|---|---|
| ANISOU | 92 | CG | TYR A | 5 | 399 | 283 | 719 | −65 | 62 | 0 | C |

| | | | | | | | | | | |
|---|---|---|---|---|---|---|---|---|---|---|
| ATOM | 93 | CD1 | TYR A | 5 | 1.065 | 1.524 | 7.193 | 1.00 | 4.51 | C |

| | | | | | | | | | | | |
|---|---|---|---|---|---|---|---|---|---|---|---|
| ANISOU | 93 | CD1 | TYR A | 5 | 593 | 436 | 684 | −104 | 20 | −93 | C |

| | | | | | | | | | | |
|---|---|---|---|---|---|---|---|---|---|---|
| ATOM | 95 | CE1 | TYR A | 5 | 0.987 | 1.317 | 5.807 | 1.00 | 4.94 | C |

| | | | | | | | | | | | |
|---|---|---|---|---|---|---|---|---|---|---|---|
| ANISOU | 95 | CE1 | TYR A | 5 | 603 | 478 | 793 | −3 | −35 | −63 | C |

| | | | | | | | | | | |
|---|---|---|---|---|---|---|---|---|---|---|
| ATOM | 97 | CZ | TYR A | 5 | 1.747 | 0.314 | 5.224 | 1.00 | 4.30 | C |

| | | | | | | | | | | | |
|---|---|---|---|---|---|---|---|---|---|---|---|
| ANISOU | 97 | CZ | TYR A | 5 | 303 | 503 | 826 | −106 | 58 | −20 | C |

| | | | | | | | | | | |
|---|---|---|---|---|---|---|---|---|---|---|
| ATOM | 98 | OH | TYR A | 5 | 1.665 | 0.088 | 3.863 | 1.00 | 5.91 | O |

| | | | | | | | | | | | |
|---|---|---|---|---|---|---|---|---|---|---|---|
| ANISOU | 98 | OH | TYR A | 5 | 529 | 973 | 741 | −218 | 31 | 38 | O |

| | | | | | | | | | | |
|---|---|---|---|---|---|---|---|---|---|---|
| ATOM | 100 | CE2 | TYR A | 5 | 2.560 | −0.477 | 6.012 | 1.00 | 4.69 | C |

| | | | | | | | | | | | |
|---|---|---|---|---|---|---|---|---|---|---|---|
| ANISOU | 100 | CE2 | TYR A | 5 | 266 | 548 | 969 | 19 | 86 | −64 | C |

| | | | | | | | | | | |
|---|---|---|---|---|---|---|---|---|---|---|
| ATOM | 102 | CD2 | TYR A | 5 | 2.643 | −0.255 | 7.376 | 1.00 | 4.66 | C |

| | | | | | | | | | | | |
|---|---|---|---|---|---|---|---|---|---|---|---|
| ANISOU | 102 | CD2 | TYR A | 5 | 399 | 489 | 881 | 35 | 131 | −54 | C |

| | | | | | | | | | | |
|---|---|---|---|---|---|---|---|---|---|---|
| ATOM | 104 | C | TYR A | 5 | 1.447 | 0.394 | 11.777 | 1.00 | 3.63 | C |

| | | | | | | | | | | | |
|---|---|---|---|---|---|---|---|---|---|---|---|
| ANISOU | 104 | C | TYR A | 5 | 362 | 433 | 583 | −6 | −148 | −49 | C |

| | | | | | | | | | | |
|---|---|---|---|---|---|---|---|---|---|---|
| ATOM | 105 | O | TYR A | 5 | 1.291 | 1.531 | 12.234 | 1.00 | 5.86 | O |

| | | | | | | | | | | | |
|---|---|---|---|---|---|---|---|---|---|---|---|
| ANISOU | 105 | O | TYR A | 5 | 931 | 445 | 848 | 20 | −329 | 104 | O |

| | | | | | | | | | | |
|---|---|---|---|---|---|---|---|---|---|---|
| ATOM | 107 | N | LYS A | 6 | 1.944 | −0.628 | 12.472 | 1.00 | 5.06 | N |

| | | | | | | | | | | | |
|---|---|---|---|---|---|---|---|---|---|---|---|
| ANISOU | 107 | N | LYS A | 6 | 741 | 455 | 727 | −27 | −82 | −80 | N |

| | | | | | | | | | | |
|---|---|---|---|---|---|---|---|---|---|---|
| ATOM | 108 | CA | LYS A | 6 | 2.355 | −0.514 | 13.878 | 1.00 | 5.24 | C |

| | | | | | | | | | | | |
|---|---|---|---|---|---|---|---|---|---|---|---|
| ANISOU | 108 | CA | LYS A | 6 | 681 | 705 | 603 | 0 | −136 | 24 | C |

| | | | | | | | | | | |
|---|---|---|---|---|---|---|---|---|---|---|
| ATOM | 110 | CB | LYS A | 6 | 1.320 | −1.141 | 14.806 | 1.00 | 5.85 | C |

| | | | | | | | | | | | |
|---|---|---|---|---|---|---|---|---|---|---|---|
| ANISOU | 110 | CB | LYS A | 6 | 766 | 829 | 627 | 153 | −109 | −82 | C |

| | | | | | | | | | | |
|---|---|---|---|---|---|---|---|---|---|---|
| ATOM | 113 | CG | LYS A | 6 | −0.103 | −0.619 | 14.659 | 1.00 | 5.66 | C |

| | | | | | | | | | | | |
|---|---|---|---|---|---|---|---|---|---|---|---|
| ANISOU | 113 | CG | LYS A | 6 | 626 | 631 | 891 | 230 | −226 | 203 | C |

| | | | | | | | | | | |
|---|---|---|---|---|---|---|---|---|---|---|
| ATOM | 116 | CD | LYS A | 6 | −0.974 | −1.199 | 15.735 | 1.00 | 7.14 | C |

| | | | | | | | | | | | |
|---|---|---|---|---|---|---|---|---|---|---|---|
| ANISOU | 116 | CD | LYS A | 6 | 857 | 1075 | 776 | 210 | −82 | 65 | C |

| | | | | | | | | | | |
|---|---|---|---|---|---|---|---|---|---|---|
| ATOM | 119 | CE | LYS A | 6 | −2.422 | −0.755 | 15.644 | 1.00 | 7.17 | C |

| | | | | | | | | | | | |
|---|---|---|---|---|---|---|---|---|---|---|---|
| ANISOU | 119 | CE | LYS A | 6 | 654 | 1281 | 785 | −65 | −208 | 274 | C |

| | | | | | | | | | | |
|---|---|---|---|---|---|---|---|---|---|---|
| ATOM | 122 | NZ | LYS A | 6 | −3.161 | −1.044 | 16.931 | 1.00 | 7.45 | N |

| | | | | | | | | | | | |
|---|---|---|---|---|---|---|---|---|---|---|---|
| ANISOU | 122 | NZ | LYS A | 6 | 671 | 1391 | 768 | −197 | 76 | 135 | N |

| | | | | | | | | | | |
|---|---|---|---|---|---|---|---|---|---|---|
| ATOM | 126 | C | LYS A | 6 | 3.683 | −1.246 | 14.110 | 1.00 | 7.06 | C |

| | | | | | | | | | | | |
|---|---|---|---|---|---|---|---|---|---|---|---|
| ANISOU | 126 | C | LYS A | 6 | 978 | 1024 | 678 | 151 | −197 | −296 | C |

| | | | | | | | | | | |
|---|---|---|---|---|---|---|---|---|---|---|
| ATOM | 127 | O | LYS A | 6 | 4.139 | −2.040 | 13.269 | 1.00 | 7.83 | O |

| | | | | | | | | | | | |
|---|---|---|---|---|---|---|---|---|---|---|---|
| ANISOU | 127 | O | LYS A | 6 | 718 | 1383 | 872 | 225 | −120 | 49 | O |

| | | | | | | | | | | |
|---|---|---|---|---|---|---|---|---|---|---|
| ATOM | 129 | OT | LYS A | 6 | 4.277 | −1.060 | 15.189 | 1.00 | 6.96 | O |

| | | | | | | | | | | | |
|---|---|---|---|---|---|---|---|---|---|---|---|
| ANISOU | 129 | OT | LYS A | 6 | 827 | 1127 | 688 | 97 | −82 | 91 | O |

| | | | | | | | | | | |
|---|---|---|---|---|---|---|---|---|---|---|
| ATOM | 130 | O | HOH B | 1 | 6.277 | −3.895 | 12.916 | 1.00 | 15.19 | O |

| | | | | | | | | | | | |
|---|---|---|---|---|---|---|---|---|---|---|---|
| ANISOU | 130 | O | HOH B | 1 | 2142 | 2093 | 1536 | 19 | 86 | 908 | O |

| | | | | | | | | | | |
|---|---|---|---|---|---|---|---|---|---|---|
| ATOM | 133 | O | HOH B | 2 | −5.950 | −1.611 | 16.616 | 0.50 | 29.99 | O |

| | | | | | | | | | | | |
|---|---|---|---|---|---|---|---|---|---|---|---|
| ANISOU | 133 | O | HOH B | 2 | 3797 | 3797 | 3797 | 0 | 0 | 0 | O |

| | | | | | | | | | | |
|---|---|---|---|---|---|---|---|---|---|---|
| ATOM | 136 | O | HOH B | 3 | −5.596 | −2.491 | 14.331 | 1.00 | 30.00 | O |

| | | | | | | | | | | | |
|---|---|---|---|---|---|---|---|---|---|---|---|
| ANISOU | 136 | O | HOH B | 3 | 3799 | 3799 | 3799 | 0 | 0 | 0 | O |

### Example III

### Identification of Additional Amyloid Binding and/or Inhibitory Compounds and Defining a More Precise Pharmacophore

### Computational Approach of Structure-Based Design of Amyloid Inhibitors

### A. Introduction

In order to identify additional compounds that can act as amyloid binders and/or inhibitors, we started with the crystal structure described above of a fiber-forming segment of Aβ in complex with the small molecule binder Orange-G. We then computationally identified candidate compounds from very large databases of compounds which interact favorably with amyloid fibers. The top-ranking compounds were then experimentally characterized by, e.g., NMR titration, Electron Microscope (EM), and MIT cell viability experiments.

Flow charts summarizing the approach used in this Example are shown in FIGS. 14 and 15.

Step I. Determination of the Co-Crystal Structure of a Fiber-Forming Segment of Aβ in Complex with the Small Molecule Binder Orange-G

Amyloid beta was chosen as a target for inhibitor design. Amyloid beta (Aβ) is a peptide of 39-42 amino acids processed from the Amyloid precursor protein (APP), and it is most commonly known in association with Alzheimer's disease (AD). The segment ¹⁶KLVFFA²¹ (SEQ ID NO: 1) has been well studied and identified as an amyloid-forming peptide involved in the fiber core structure. As shown in Examples I and II above, we have determined the crystal structure of this fiber-forming segment in complex with the small molecule binder Orange-G (see, e.g., FIG. 1).

Step II. Select Compounds from Compound Databases

We selected compounds for docking from two choices of purchasable compound libraries of compounds:

### 1) Cambridge Structure Database (CSD) Set

102,236 organic compounds having crystal structures with R-factor of better than 0.1 were extracted from the Cambridge Structure Database (version 5.32, November 2010) using ConQuest. The SMILES string of each structure was then used to locate its purchasing information among the ZINC purchasable set (http://zinc.docking.org/) by OpenBabel package (http://openbabel.org/). The fast index table of all SMILES strings of the ZINC purchasable set was generated to allow the fast search of each CSD structure against ZINC purchasable set. CSD structures failed in locating their purchasing information (that is, without any hit in searching against ZINC purchasable set) were omitted. A library of 11,057 compounds was finally compiled. A total of 13,918 structures from CSD representing 11,057 compounds were compiled, whose purchasing information is annotated by ZINC purchasable database. The information of CSD code and ZINC entry can be downloaded from the world wide web site people.mbi.ucla.edu/jiangl/AmyloidInhibitor Paper.

### 2) Flat Compound (FC) Set

A library of 6,589 compounds containing phenol and less than 3 freely rotatable bonds were extracted from the ZINC database (http://zinc.docking.org/). Those compounds have a common feature of planar aromatic ring, a so called “flat” compound. The flat compound library includes those compounds which have similar chemical structures to naturally fibril-binding molecules, for instance, Thioflavin-T (ThT), Congo Red, Green tea epigallocatechin-3-gallate (EGCG) and Curcumin. And it also includes many natural phenols, such as gallic acid, ferulic acid, coumaric acid, propyl gallate, epicatechin, epigallocatechin, epigallocatechin gallate, and etc. The complete list of ZINC entries of these compounds can be downloaded from the world wide web site people.mbi.ucla.edu/jiangl/AmyloidInhibitor Paper.

### Ligand Library Preparation

From these two compound libraries, each molecule was then prepared. Hydrogens of each molecule were added if there is any missing hydrogen by using the program Omega (v. 2.3.2, OpenEye). Ligand atoms were represented by the most similar Rosetta atom type, their coordinates were re-centered to the origin, and their partial charges were assigned by OpenEye's AMI-BCC implementation. The ligand perturbation ensemble near the crystal conformation (CSD set) or starting conformation (FC set) of each was then generated. For each rotatable bond of the ligand, small degree torsion angle deviation) (+/−5° was applied. K-mean clustering method was used to generate the ligand perturbation ensemble and similar/redundant conformation (rmsd to the selected conformation is less than 0.5 Å) was omitted. Finally, up to 100 conformations for each ligand were generated, and ready for Rosetta LigandDock.

Step III. Rosetta LigandDock with Additional Near “Native” Perturbation Sampling

We developed a general approach for docking a large library of commercial compounds onto the flat surface of the amyloid fiber. Starting from the template of the ¹⁶KLVFFA²¹ (SEQ ID NO: 1)/Orange-G structure described above, we computationally identified small molecule inhibitors that bind the side of the ¹⁶KLVFFA²¹ (SEQ ID NO: 1) fiber.

The docking algorithm is similar to the method previously described in the RosettaLigand docking paper (J Mol. Biol. 2009 Jan. 16; 385(2):381-92. Epub 2008 Nov. 18.), following the same three stages: coarse-grained stage, Monte Carlo minimization (MCM) stage and gradient-based minimization stage. The original RosettaLigand method performed a full sampling of the ligand internal and protein side-chain degrees of freedom in. In order to enable the fast run time required by any screening method, we sampled the ligand and protein side-chain torsion angles in near-“native” perturbation fashion, where only the near-“native” conformation of side-chain and ligand rotamers were allowed and any conformation far away from the starting conformation were omitted. For each protein side-chain, the deviations (+/−0.33, 0.67, 1 sd) around each input torsion was applied based on the standard deviation value of the same torsion bin from the backbone-dependent Dunbrack rotamer library. For each internal torsion of the ligand, the deviations) (+−5° around the input torsion was applied as described above. This near-“native” perturbation sampling makes count for both high-resolution finer sampling around the starting conformation and fast speed required by screening a large library.

A summary of the method of structure-based selection/identification of small compound inhibitors of Aβ is shown in FIG. 15. In step I, the crystal structure is determined of a co-crystal of an amyloid-like segment ¹⁶KLVFFA²¹ (SEQ ID NO: 1) of Aβ with an Amyloid-binding Ligand X. Panel a shows the pharmacophore, where the Ligand X (the template molecule we choose here is an Amyloid-binding molecule, Orange G, shown in orange sticks) binds to the side of KLVFFA (SEQ ID NO: 1) fibers. In step II, we docked a library of ˜18 thousand commercially available compounds into the chemical environment of Ligand X. Panel b shows the overlay of representative high-ranking compounds as judged by a good fit at the binding interface with a strong binding energy and tight shape complementary score. In step III, those top-ranking compounds were filtered by docking against the fibril structure of full-length Aβ (Tycko's ssNMR model, pdb entry 2LMO). The “flat” compounds with better binding energy and shape complementary than that of the template molecule Orange-G having a good fit with Aβ fiber, which make hydrogen bonds with the side chains of lysine 16 and stick to the side of fibrillar beta sheet for both KLVFFA (SEQ ID NO: 1) and Aβ fibers, were picked up for the further human inspection. Finally 35 compounds (referred to as “BAF” compounds), were selected for experimental characterization and validation, including NMR titration, Electron Microscope (EM) and MTT cell viability experiments (step IV).

### Step IV—Characterization of the Compounds

### Step IVA. MTT Cell Proliferation/Viability Assay

We tested candidate compounds by the MTT-based cell proliferation/viability assay, as described on the ATCC web site. Briefly, the ATCC MTT Cell Proliferation Assay quantitates the reduction of the yellow tetrazolium salt (MTT) in response to an external factor, such as treatment with a compound of the present invention, as a measure of a cell population's response to the external factor. The assay measures the cell proliferation rate and conversely, when metabolic events lead to apoptosis or necrosis, the reduction in cell viability. In our tests, Hela and PC12 cell lines were used to assess the toxic effect of Abeta protein. Abeta at 0.5 μM was a positive control. The small molecule inhibitors were added to samples with different concentrations (such as 2.5 μM). After 12 h incubation at room temperature, the absorbance of reduced MIT was measured at 570 nm. Each of the experiments was repeated 3 times with 4 replicates per sample per concentration. Our MTT cell viability assay quantified the percentage of survival cells upon the treatment of the mixture of Abeta and compound inhibitors. The rescuing percentage of each compound was calculated by normalizing the survival percentage using the buffer-treated cell as 100% viability and Abeta-treated cell as 0% viability.

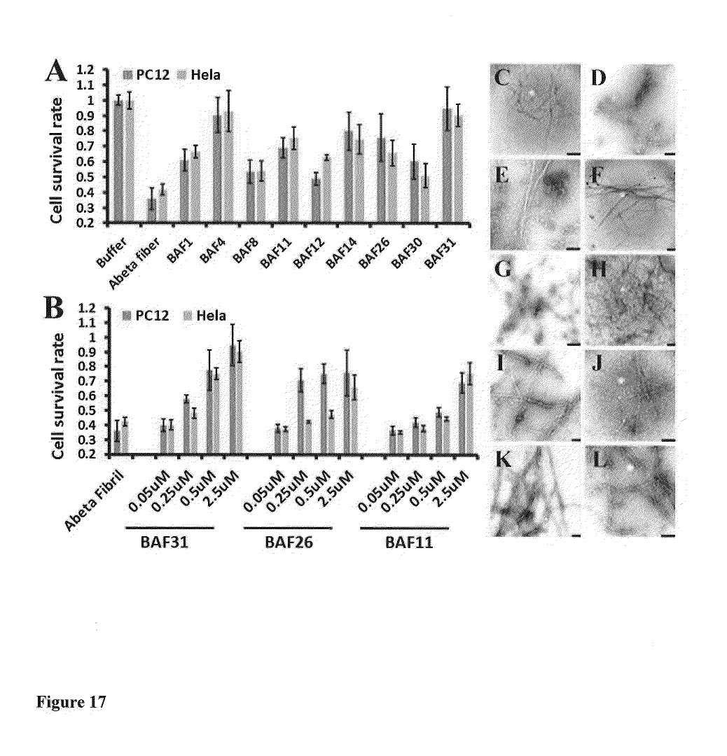

FIG. 17 shows the results of the MTT assays and electronmicroscopy (EM) studies. Representative compounds BAF31, BAF26 and BAF11 are shown to reduce Aβ cytotoxicity in a dose dependent manner. EM studies show that all of the tested compounds which inhibit Aβ toxicity do not inhibit Aβ fibrillation.

These studies support the proposed model shown in FIG. 16, which suggests that soluble aggregation intermediates such as amyloid oligomers are more toxic than amyloid fibers, while fibrils may serve as reservoirs of toxic oligomers. In this suggested model, fiber-binding molecules can inhibit amyloid toxicity by shifting the equilibrium from toxic oligomers towards end-stage fibers.

Nine compounds were shown to inhibit Aβ toxicity. These compounds are listed in Table 7. Of these, seven compounds have never been reported to inhibit Aβ toxicity. The structures of these seven compounds are shown in FIG. 18.

### Step IVB. Expanding the Set of Compounds to Include Derivatives

We studied derivatives/homologs of the compounds we determined to be active. These derivatives are listed in Table 8.

The results of activity studies of some representative compounds, BAF11 and BAF30, and the active derivatives thereof are shown in FIGS. 19 and 20, respectively. For BAF11, the compound and the derivatives the Isomer, σR1, σR3 and ΔOHσR are active; and for BAF30, the compound and the derivative σR1 are active.

### Step IVC. Derive a Refined Model of the Amyloid Pharmacophore, Based on the Overlay of Structural Models of the Active Compounds

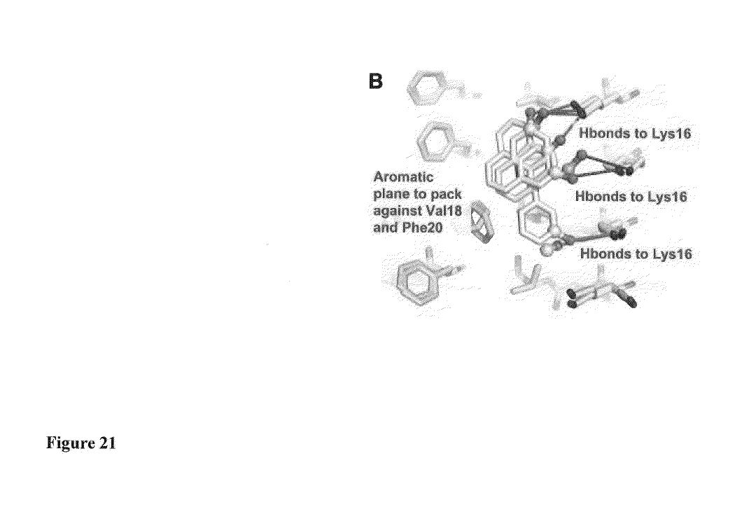

FIG. 21 shows an amyloid pharmacophore, derived based on the overlay of structural models of the active compounds described herein. The hydrogen-bonding and hydrophobic interactions described in this pharmacophore match well with the crystal structures of KLVFFA (SEQ ID NO: 1) fiber and Orange-G: the designed molecules bind specifically to lysine (Lys16) side chains of adjacent Abeta sheets via hydrogen bonds or salt bridges, and their planar aromatic portion packs against apolar residues (phenylalanine 20 and valine 18) from Abeta sheets. By creating a tight, low energy interface across several peptide fiber strands, this fibril-binding molecule apparently stabilizes fiber structure and thus inhibits Abeta toxicity.

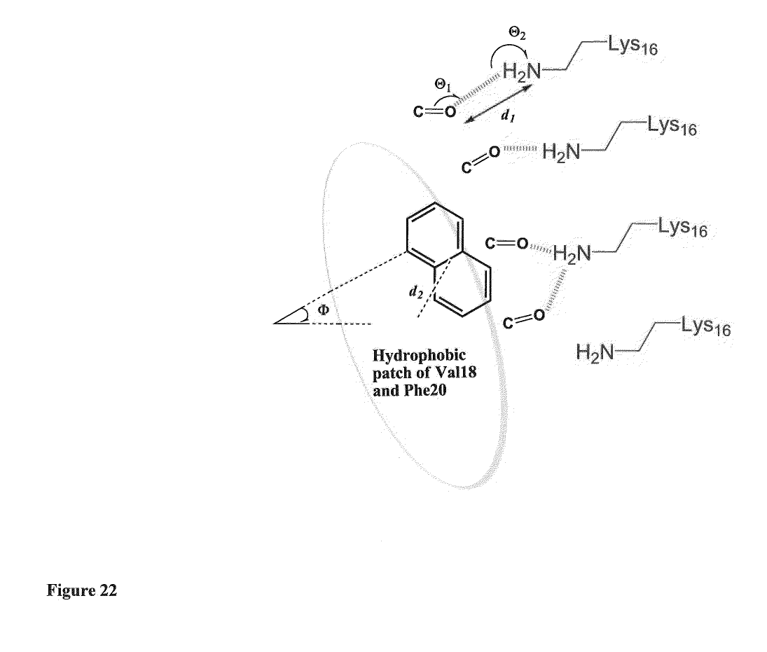

The geometries defined in this amyloid pharmacophore are highlighted in FIG. 22. In the figure, the carbonyl group is used to represent the H-bond acceptor (or negative charge) of the inhibitor, and the naphthalene ring is used to represent the planar aromatic portion of the inhibitor.

The defined interactions and geometries are:

1) H-bond acceptor (or negative charge) of the inhibitor should make either a hydrogen bond or a salt bridge to the sidechain nitrogen atoms (NZ) of at least two Lysine 16 redidues from adjacent Abeta sheets. Our data suggest that the active inhibitors should bind across 2 to 4 adjacent Abeta strands.
2) The hydrogen bond or salt bridge described in 1) should follow the general rule of H-bond geometry. They are:
a) distance (d₁, as show in the figure) between the NZ atom of Lys16 and inhibitor H-bond acceptor atoms: 2.8˜3.5 angstrom;
b) angle (Θ₁) at inhibitor H-bond acceptor atoms: 100˜150°
c) angle (Θ₂) at the NZ atom of Lys16: 130˜180°;
   3) Hydrophobic interactions between the apolar residues (phenylalanine (Phe) 18 and valine (Val) 20) and the planar aromatic portion of the compounds. The aromatic portion of compounds should be planar or semi-planar to pack against the flat surface of Abeta which runs across at least 2 adjacent Abeta sheets.
   4) The hydrophobic interactions described in 3) should follow the pi-pi stacking geometry. It is:
a) distance (d₂) between sidechain center of the apolar residues and the center of compound aromatic rings: 4.0˜5.0 angstrom;
b) dihedral angle (Φ) between the surface plan defined by Phe18 and Val20 and the aromatic ring of the compounds: 0˜40°.

### Experimental Validation of Our Computational Approach by NMR Studies

As a validation of our computational approach, we used nuclear magnetic resonance (NMR) to characterize the interactions between our BAF compounds and KLVFFA (SEQ ID NO: 1) and Aβ fibers. First the ¹H NMR spectra for two representative compounds (BAF1 & BAF8) and the binder molecule Orange-G were collected in the presence of increasing concentrations of KLVFFA (SEQ ID NO: 1) fibers. By monitoring the BAF1 compound peak area over a range of KLVFFA (SEQ ID NO: 1) fiber concentrations, we estimate the apparent dissociation constant (Kd) value of the interaction of BAF1 with KLVFFA (SEQ ID NO: 1) fibers to be ˜12 μM. We also performed an NMR titration experiment for BAF8 and obtained a binding affinity Kd of ˜24 μM. Since our computational approach identified candidate molecules that have a stronger predicted binding affinity than that of our template molecule Orange-G, we then measured the apparent Kd of Orange-G (˜43 μM). The weak binding affinity of Orange-G confirmed the success of our computational approach.

### ¹H Nuclear Magnetic Resonance Sample Preparation and Measurements.

NMR samples contained 550 μL of designed compounds were added from 1 mM stocks in H₂O to a final concentration of 100 μM. Fibrillar KLVFFA (SEQ ID NO: 1) and Abeta were added at the indicated concentrations. 500 MHz ¹H NMR spectra were collected on a Bruker DRX500 at 283 K. H₂O resonance was suppressed through presaturation. Spectra were processed with XWINNMR 3.6.

### Conclusion

We used NMR spectroscopy to validate the direct binding of designed compounds to Abeta fibers. Moreover, Electron Microscope (EM) studies showed those designed compounds cannot inhibit fibrillation of Abeta. Those compounds showed inhibition of the Abeta toxicity in mu cell viability assays. The ability of their derivative/variant molecules to reduce Abeta toxicity correlated well with our structural models (crystal structure and docked models), and those data of cell viability allow us to derive the precise model of an “amyloid pharmocophore”. Supporting our hypothesis, our results showed that the designed compounds bind to amyloid proteins and greatly inhibit amyloid toxicity, indicating that these agents will likely be effective therapeutic and/or diagnostic agents for amyloid disease.

TABLE 7
Detailed list of the active BAF compounds for the step IV A.

| | | | | | Rescuing | | | |
|---|---|---|---|---|---|---|---|---|
| | Molecular | Molecular | Sources/ | | Percentage^{d}(%) | | ZINC entry | |
| Compound | Formula | Weight^{a} | Purchasing | Purity | PC12 | Hela | code^{e} | SMILES String |
| BAF1 | C₂₀H₈Br₄O₅ | 647.9 | Sigma- | ~99% | 44.3 ± | 41.3 ± | ZINC04261875 | c1ccc2c(c1)C(═O)OC23c4ccc(c |
| | | | Aldrich | | 9.8 | 7.1 | | (c40c5c3ccc(c5Br)O)Br)O |
| BAF4 | C₂₄H₁₆N₂O₆ | 428.4 | Aldrich | ≧95% | 89.2 ± | 87.7 ± | ZINC13346907 | c1cc(c(cc1O)O)c2cc3c(cc2N)o |
| | | | | | 8.3 | 4.8 | | c- |
| | | | | | | | | 4cc(═O)c(cc4n3)c5ccc(cc5O)O |
| BAF8 | C₁₇H₁₄N₂O₅S | 358.4 | Sigma- | ≧90% | 25.1 ± | 27.1 ± | ZINC12358966 | Cc1ccc(c(c1)/N═N/c2c3ccccc3 |
| | | | Aldrich | | 7.4 | 4.5 | | c(cc2O)S(═O)(═O)[O—])O |
| BAF11 | C₂₀H₁₃N₂O₅S | 393.5 | NCI plated | ^{b} | 56.6 ± | 45.9 ± | ZINC04521479 | c1ccc2c(c1)ccc(c2O)/N═N/c3c |
| | | | 2007 | | 6.8 | 3.5 | | 4ccccc4c(cc3O)S(═O)(═O)\|O—] |
| BAF12 | C₁₃H₈Br₃NO | 433.9 | NCI plated | ^{b} | 27.0 ± | 24.9 ± | ZINC12428965 | c1cc(ccc1/N═C/c2cc(cc(c2O) |
| | | | 2007 | | 5.8 | 2.9 | | Br)Br)Br |
| BAF14 | C₁₇H₁₀O₄ | 278.3 | Aldrich | [symbol] | 82.5 ± | 70.0 ± | ZINC05770717 | c12c(cc(cc1)C(═O)C═O)Cc1c2c |
| | | | | | 6.5 | 2.9 | | cc(c1)C(═O)C═O |
| BAF26 | C₁₅H₁₀O₈ | 318.2 | Sigma | ≧96% | 85.5 ± | 51.4 ± | ZINC03874317 | c1c(cc(c(c1O)O)O)c2c(c(═O)c |
| | | | | | 15.1 | 4.5 | | 3c(cc(cc3o2)O)O)O |
| BAF30 | C₁₄H₈O₅ | 256.2 | Aldrich | ^{c} | 54.9 ± | 19.3 ± | ZINC03870461 | c1cc2c(cc1O)C(═O)c3c(ccc(c3 |
| | | | | | 9.6 | 8.4 | | O)O)C2═O |
| BAF31 | C₁₉H₂₁NO₃ | 311.4 | Sigma | ≧98% | 92.6 ± | 83.0 ± | ZINC03874841 | CCCN1CCC2═C3C1CC4═C(C3═ |
| | | | | | 25.7 | 5.8 | | CC(═C2)O)C(═C(C═C4)O)O |

^{a}molecule weight (anhydrous basis) excluding the salt and water molecules
^{b} with the standard of NCI free compound library
^{c} analytical data for Aldrich^{CPR} products are not available
^{d}rescue percentage is a scaled cell survival rate
^{e}entry code for the ZINC database (http: //zinc.docking.org) [symbol]
indicates data missing or illegible when filed

TABLE 8
List of the selected active BAF compounds and their derivatives for the step IV B.

| | Molecular | Molecular | | Rescuing | ZINC entry/ |
|---|---|---|---|---|---|
| Compound | Formula | Weight | Description | Percentage (%) | Catalog No. |
| BAF31 | C₁₉H₂₁NO₃ | 311 | | 83.0 ± 5.8 | ZINC03874841 |
| BAF31ΔOH | C₁₉H₂₁NO₂ | 295 | remove one hydroxyl (OH) | 14.9 ± 2.3 | ZINC03874841 |
| BAF26 | C₁₅H₁₀O₈ | 318 | | 51.4 ± 4.5 | ZINC03874317 |
| BAF26ΔOH^{A} | C₁₅H₁₀O₇ | 302 | remove one OH from loc A | 40.3 ± 5.8 | ZINC03869685 |
| BAF26ΔOH^{AB} | C₁₅H₁₀O₆ | 286 | remove two OHs from loc A, B | 15.3 ± 5.5 | ZINC03869768 |
| BAF26ΔOH^{AB}αOH^{D} | C₁₅H₁₀O₇ | 302 | remove two OHs from loc A, B; | 43.6 ± 6.6 | ZINC03881558 |
| | | | add one OH at loc D | | |
| BAF26ΔOH^{AC} | C₁₅H₁₀O₆ | 286 | remove two OHs at loc A, C | 33.4 ± 8.7 | ZINC18185774 |
| BAF26ΔOH^{AC}αOR1^{C} | C₂₁H₂₀O₁₂ | 464 | remove one OHs from loc A; | 31.0 ± 5.0 | ZINC03973253 |
| | | | replace OH with OR group at loc C | | |
| BAF26ΔOH^{AC}αOR2^{C} | C₂₇H₃₀O₁₆ | 611 | remove one OHs from loc A; | 33.0 ± 6.8 | Error! |
| | | | replace OH with OR group at loc C | | Hyperlink |
| | | | | | reference not |
| | | | | | valid. |
| BAF26ΔOH^{ABC} | C₁₅H₁₀O₅ | 270 | remove three OHs from loc A, B, C | 0.1 ± 2.9 | ZINC03871576 |
| BAF26^{RED} | C₁₅H₁₄O₇ | 306 | the reduction form of BAF26 | 17.7 ± 6.5 | ZINC03870336 |
| BAF30 | C₁₄H₈O₅ | 256 | | 28.0 ± 8.4 | ZINC03870461 |
| BAF30αR | C₂₂H₂₀O₁₃ | 492 | add additional group away from | 20.0 ± 9.5 | ZINC28095922 |
| | | | binding interface | | |
| BAF30σOH^{A}αOH | C₁₄H₈O₆ | 272 | change one OH (A) position; add | 8.6 ± 9.2 | ZINC03874832 |
| | | | another OH | | |
| BAF30σOH^{A}ΔOH^{B}αCOO | C₁₅H₈O₆ | 284 | move one OH (A) position; delete | 9.0 ± 3.4 | ZINC04098704 |
| | | | an OH from loc B; add a carboxyl | | |
| BAF30σOH^{AB}αCH₃ | C₁₅H₁₀O₅ | 270 | move one OH (A) position; delete | 6.5 ± 1.4 | ZINC03824868 |
| | | | an OH from loc B; add a carboxyl | | |
| BAF11 | C₂₀H₁₃N₂O₅S | 393 | | 45.9 ± 3.5 | ZINC04521479 |
| BAF11^{ISO} | C₂₀H₁₃N₂O₅S | 393 | isomer form of BAF11 | 33.2 ± 5.0 | ZINC12405071 |
| BAF11σR1 | C₂₀H₁₄N₄O₈S₂ | 502 | change the auromatic group | 35.2 ± 9.4 | ZINC25558261 |
| BAF11σR2 (BAF8) | C₁₇H₁₄N₂O₅S | 358 | change the auromatic group | 27.1 ± 4.5 | ZINC12358966 |
| BAF11σR3 | C₁₆H₁₂N₂O₆S | 360 | change the auromatic group | 27.9 ± 3.6 | ZINC04900892 |
| BAF11αNO₂⁻ | C₂₀H₁₂N₃O₇S | 438 | add charged group (nitro) | 14.9 ± 6.0 | ZINC16218542 |
| BAF11^{ISO}αCOO⁻ | C₂₁H₁₂N₂O₇S | 436 | BAF11 isomer; add charged group | 5.8 ± 5.2 | ZINC03861030 |
| | | | (carboxyl) | | |
| BAF11^{ISO}αSO₃⁻ | C₂₀H₁₁N₂O₁₁S₃ | 552 | BAF11 isomer; add charged group | 1.8 ± 5.3 | SIGMA-33936 |
| | | | (sulfate) | | |
| BAF11ΔOHσR | C₂₀H₁₄N₂O₄S | 378 | remove an OH; change the position | 15.4 ± 4.3 | ZINC04803992 |
| | | | of the auromatic group | | |
| BAF11ΔOHαSO₃⁻ | C₂₀H₁₄N₂O₇S₂ | 458 | remove an OH; add sulfate group | 11.6 ± 3.4 | ZINC03954029 |
| BAF11ΔOHαR1 | C₂₀H₁₈N₄O₅S | 426 | remove an OH; add additional | 11.5 ± 6.1 | ZINC04416667 |
| | | | group to the auromatic ring | | |
| BAF11σOHαR2 | C₂₄H₂₀N₄O₄S | 461 | swap the poistion of the OH and | 4.6 ± 4.8 | ZINC04804174 |
| | | | auromatics | | |
| BAF11σOHαR3 | C₁₆H₁₉N₃O₅S | 365 | swap the poistion of the OH and | 3.8 ± 5.7 | ZINC17378758 |
| | | | auromatics | | |

TABLE 9
List of all tested BAF compounds

| | | | | Rescuing | |
|---|---|---|---|---|---|
| | Molecular | Molecular | Sources/ | Percentage | |
| Compound | Formula | Weight | Purchasing | (%) | ZINC entry |
| BAF1 | C₂₀H₈Br₄O₅ | 648 | Sigma-Aldrich | 41.3 ± 7.1 | ZINC04261875 |
| BAF2 | C₁₉H₁₄O₅S | 354 | Sigma-Aldrich | 4.0 ± 3.2 | ZINC03860918 |
| BAF3 | C₁₆H₁₃NO₃ | 267 | Ryan Scientific | 4.4 ± 4.7 | ZINC04289063 |
| BAF4 | C₂₄H₁₆N₂O₆ | 428 | Aldrich | 87.7 ± 4.8 | ZINC13346907 |
| BAF5 | C₁₆H₇Na₃O₁₀S₃ | 524 | Sigma-Aldrich | 10.8 ± 6.8 | ZINC03594314 |
| BAF6 | C₂₆H₂ON₂ | 360 | Alfa-Aesar | 5.1 ± 6.5 | ZINC08078162 |
| BAF7 | C₁₈H₁₂N₆ | 312 | Alfa-Aesar | 2.2 ± 1.8 | ZINC00039221 |
| BAF8 | C₁₇H₁₄N₂O₅S | 358 | Sigma-Aldrich | 27.1 ± 4.5 | ZINC12358966 |
| BAF9 | C₁₉H₁₃N₃O₄S | 379 | NCI plated 2007^{a} | −3.3 ± 21.9 | ZINC03954432 |
| BAF10 | C₁₇H₁₃NO₃ | 279 | NCI plated 2007 | 3.2 ± 4.9 | ZINC00105108 |
| BAF11 | C₂₀H₁₃N₂O₅S | 393 | NCI plated 2007 | 45.9 ± 3.5 | ZINC04521479 |
| BAF12 | C₁₃H₈Br₃NO | 434 | NCI plated 2007 | 26.1 ± 2.9 | ZINC12428965 |
| BAF13 | C₁₉H₁₆ClNO₄ | 358 | Sigma-Aldrich | 0.3 ± 2.1 | ZINC00601283 |
| BAF14 | C₁₇H₁₀O₄ | 278 | Aldrich | 70.0 ± 2.9 | ZINC05770717 |
| BAF15 | C₂₃H₂₈O₈ | 432 | Sigma-Aldrich | 12.8 ± 4.3 | ZINC00630328 |
| BAF16 | C₁₉H₁₉NO₅ | 341 | Sigma-Aldrich | 5.3 ± 7.8 | ZINC28616347 |
| BAF17 | C₂₃H₂₅N₅O₂ | 404 | Sigma-Aldrich | 5.5 ± 3.4 | ZINC00579168 |
| BAF18 | C₂₄H₁₆O₂ | 336 | ChemDiv | 5.6 ± 2.2 | ZINC02168932 |
| BAF19 | C₁₈H₁₄N₂O₆ | 354 | ChemDiv | 3.1 ± 4.1 | ZINC01507439 |
| BAF20 | C₂₅H₁₉N₅OS | 438 | ChemDiv | 7.6 ± 4.4 | ZINC15859747 |
| BAF21 | C₁₉H₁₄Br₂O | 418 | ChemDiv | 6.1 ± 3.2 | ZINC38206526 |
| BAF22 | C₂₁H₁₆N₂O₃S₂ | 408 | Life Chemicals | 2.8 ± 4.7 | ZINC04496365 |
| BAF23 | C₁₆H₁₁ClO₅S | 351 | Enamine Ltd | 2.8 ± 5.4 | ZINC02649996 |
| BAF24 | C₂₃H₁₉NO₃ | 357 | Sigma-Aldrich | 15.6 ± 4.5 | ZINC03953119 |
| BAF25 | C₁₄H₈Cl₂N₄ | 303 | Sigma-Aldrich | 3.5 ± 2.6 | ZINC00403224 |
| BAF26 | C₁₅H₁₀O₈ | 318 | Sigma | 51.4 ± 4.5 | ZINC03874317 |
| BAF27 | C₂₁H₁₆BrN₃O₆ | 486 | ChemBridge | 3.7 ± 1.0 | ZINC01208856 |
| BAF28 | C₁₇H₁₂N₂O₃ | 292 | ChemBridge | 1.7 ± 4.0 | ZINC00061083 |
| BAF29 | C₂₂H₁₀N₄O₂ | 362 | ChemBridge | 0.5 ± 5.3 | ZINC00639061 |
| BAF30 | C₁₄H₈O₅ | 256 | Aldrich | 19.3 ± 8.4 | ZINC03870461 |
| BAF31 | C₁₉H₂₁NO₃ | 311 | Sigma | 83.0 ± 5.8 | ZINC03874841 |
| BAF32 | C₁₅H₁₄O₇ | 306 | Sigma-Aldrich | 17.7 ± 6.5 | ZINC03870336 |
| BAF33 | C₂₇H₃₃N₃O₈ | 528 | Sigma-Aldrich | 7.1 ± 2.0 ^{b} | SIGMA-R2253^{c} |
| BAF34 | C₃₀H₁₆N₄O₁₄S₄ | 785 | Aldrich | | ALDRICH- |
| | | | | | S432830^{c} |
| BAF35 | C₁₀H₆S₂O₈ | 318 | Sigma-Aldrich | 3.3 ± 3.4 | ZINC01532215 |
| Orange-G | C₁₆H₁₂N₂O₇S₂ | 408 | Sigma-Aldrich | −2.3 ± 7.3 | ZINC04261935 |

^{a}National Cancer Institute (NCI) free compound library (http://dtp.nci.nih.gov/)
^{b} The toxicity results of BAF34 were not consistent for several independent replica experiments, which can be due to the possible impurity and the large molecule weight of the compound.
^{c}ZINC entry of the compound is not applicable, and the catalog number from Sigma-Aldrich is provided.

### Example IV

### Testing for Efficacy of Compounds of the Invention in Animal Models

Compounds of the invention that are shown to protect human cells in vitro from the toxic effects of Abeta and/or tau will be tested in art-recognized animal models, including in D. melanogaster, C. elegans, and mice. Examples of transgenic animals constructed to exhibit amyloid disease include the Drosophila flies; mice produced by Jackson et al. (A Genomic Screen for Modifiers of Tauopathy Identifies Puromycin-Sensitive Aminopeptidase as an Inhibitor of Tau-Induced Neurodegeneration (2006) Neuron 51, 549-560); and the Abeta expressing mice of G. Cole et al. (Science, 274, 99-102 (1996)). Other suitable models will also be evident to skilled workers.

Compounds will be administered to the test animals by conventional methods, depending on the nature of the compounds, e.g. by adding them to the animals' food, injecting intravenously, or administering (pumping) directly into the spinal column or brain. The animals will be monitored for the effect of the compounds on suitable characteristics of the amyloid disease, compared to suitable controls. Details of such protocols are standard in the art, and will be well-known to those of skill in the art.

It is expected that the compounds being tested will elicit a reduction of symptoms or manifestations of the disease or condition.

From the foregoing description, one skilled in the art can easily ascertain the essential characteristics of this invention, and without departing from the spirit and scope thereof, can make changes and modifications of the invention to adapt it to various usage and conditions and to utilize the present invention to its fullest extent. The preceding preferred specific embodiments are to be construed as merely illustrative, and not limiting of the scope of the invention in any way whatsoever. The entire disclosure of all applications, patents, and publications (including U.S. provisional application 61/507,810, filed Jul. 14, 2010), particularly with regard to the specific disclosure for which they are referenced herein cited above, and in the figures, are hereby incorporated in their entirety by reference.

Abeta is a cleavage product of the precursor protein APP with UniProt accession code P05067 (A4_HUMAN). Among the best-studied peptide products are Abeta-40 and Abeta-42. A skilled worker will know the sequence of a variety of forms of Abeta that are suitable for use in the present invention. A representative sequence, of Abeta-42, is referred to herein as SEQ ID NO:21:

>sp|P05067|672-713 (Abeta 1-42)
DAEFRHDSGYEVHHQKLVFFAEDVGSNKGAIIGLMVGGVVIA

The sequence of the human tau protein (SEQ ID NO:22), is according to the document isoform of tau with the UniProt accession code P10636 (TAU_HUMAN). This sequence is referred to herein as SEQ ID NO:22:

>sp|P10636|TAU_HUMAN Microtubule-associated
protein tau
MAEPRQEFEVMEDHAGTYGLGDRKDQGGYTMHQDQEGDTDAGLKESPLQT
PTEDGSEEPGSETSDAKSTPTAEDVTAPLVDEGAPGKQAAAQPHTEIPEG
TTAEEAGIGDTPSLEDEAAGHVTQEPESGKVVQEGFLREPGPPGLSHQLM
SGMPGAPLLPEGPREATRQPSGTGPEDTEGGRHAPELLKHQLLGDLHQEG
PPLKGAGGKERPGSKEEVDEDRDVDESSPQDSPPSKASPAQDGRPPQTAA
REATSIPGFPAEGAIPLPVDFLSKVSTEIPASEPDGPSVGRAKGQDAPLE
FTFHVEITPNVQKEQAHSEEHLGRAAFPGAPGEGPEARGPSLGEDTKEAD
LPEPSEKQPAAAPRGKPVSRVPQLKARMVSKSKDGTGSDDKKAKTSTRSS
AKTLKNRPCLSPKHPTPGSSDPLIQPSSPAVCPEPPSSPKYVSSVTSRTG
SSGAKEMKLKGADGKTKIATPRGAAPPGQKGQANATRIPAKTPPAPKTPP
SSGEPPKSGDRSGYSSPGSPGTPGSRSRTPSLPTPPTREPKKVAVVRTPP
KSPSSAKSRLQTAPVPMPDLKNVKSKIGSTENLKHQPGGGKVQIINKKLD
LSNVQSKCGSKDNIKHVPGGGSVQIVYKPVDLSKVTSKCGSLGNIHHKPG
GGQVEVKSEKLDFKDRVQSKIGSLDNITHVPGGGNKKIETHKLTFRENAK
AKTDHGAEIVYKSPVVSGDTSPRHLSNVSSTGSIDMVDSPQLATLADEVS
ASLAKQGL

1. A method for determining on a computer the relevant criteria for designing or selecting on a computer a small molecule amyloid binder or inhibitor, comprising
a) co-crystallizing a protofilament of an amyloid protein with a small molecule that is known to bind to the amyloid protein; and
b) determining on a computer the three-dimensional structure of the co-crystal, thereby determining the atomic coordinates of the binding surface or binding pocket

2. The method of claim 1, further comprising designing or selecting on a computer a small molecule amyloid binder or inhibitor, comprising
a) docking test compounds to the crystal structure determined in b) on a computer, and
b) selecting test compounds which exhibit a calculated binding energy below that of the small molecule used to form the co-crystal made in a), as candidate amyloid binders.

3. The method of claim 1, wherein the amyloid protein is Aβ, and the small molecule is a charged or polar molecule comprising one or more flat aromatic rings.

4. The method of claim 3, wherein the charged or polar molecule is Orange-G.

5. The method of claim 2, wherein the atomic coordinates of the three-dimensional structure are shown in Table 3, and the amino acid residues of the amyloid molecule which contact the amyloid binder are selected from one or more of Lys16, Leu17, Val18, Phe19, or Phe20, or combinations thereof.

6. The method of claim 1, wherein the amyloid protein is tau, and the small molecule is a charged or polar molecule comprising one or more flat aromatic rings.

7. The method of claim 6, wherein the charged or polar molecule is Orange-G.

8. The method of claim 7, wherein the atomic coordinates of the three-dimensional structure are shown in Table 4, and the amino acid residues of the amyloid molecule which contact the amyloid binder are selected from one or more of Gln2, Val4, or Lys6, or combinations thereof.

9. The method of claim 1, wherein the amyloid protein is tau, and the small molecule is an elongated apolar molecule.

10. The method of claim 9, wherein the elongated apolar molecule is curcumin or DDNP.

11. The method of claim 10, wherein the atomic coordinates of the three-dimensional structure are shown in Table 5 or 6, and the amino acid residues of the amyloid molecule which contact the amyloid binder are selected from one or more of Val1, Gln2, Ile3, Val4, Tyr5 or Lys6, or combinations thereof.

12. A method for designing or selecting on a computer a candidate small molecule amyloid binder or inhibitor, comprising
a) docking test compounds to the binding site or binding surface determined from the three-dimensional structure of a co-crystal of a protofilament of an amyloid protein bound to a small molecule which is known to bind to the amyloid protein, wherein the atomic coordinates of the binding site or binding surface are as set forth in the following Tables 3-6, and amino acid residues of the amyloid molecule which contacts the amyloid binder are as indicated:
(i) Table 3 (based on an Orange-G/Aβ co-crystal), wherein the amino acid residues of the amyloid molecule are selected from one or more of Lys16, Leu17, Val18, Phe19, or Phe20, or combinations thereof; or
(ii) Table 4, (based on an Orange-G/tau co-crystal), wherein the amino acid residues of the amyloid molecule are selected from one or more of Gln2, Val4, or Lys6, or combinations thereof; or
(iii) Table 5 (based on a co-crystal of tau with curcumin), wherein the amino acid residues of the amyloid molecule are selected from one or more of Val1, Gln2, Ile3, Val4, Tyr5 or Lys6, or combinations thereof;
(iv) Table 6 (based on a co-crystal of tau with DDNP), wherein the amino acid residues of the amyloid molecule are selected from one or more of Val1, Gln2, Ile3, Val4, Tyr5 or Lys6, or combinations thereof;
(b) selecting test compounds which exhibit an energy below that of the small molecule used to form the co-crystal made in a), as candidate amyloid binders.

13. The method of claim 2, wherein the docking is accomplished by a docking program in which the test molecule and protein side chain torsion angles and small molecule rotamers are sampled in a near native perturbation fashion.

14. The method of claim 2, further comprising testing the candidate amyloid binders for their ability to inhibit amyloid-mediated cell toxicity, and identifying and selecting candidate amyloid inhibitors which inhibit amyloid-mediated cell toxicity to a greater degree than the small molecule which was co-crystallized with the amyloid.

15. The method of claim 2, further comprising characterizing and validating the candidate binders by X-ray crystallography, NMR spectroscopy (titration), ITC (isothermal titration calorimetry), thermal denaturation, mass spectrography, or SPR (surface plasmon resonance), to measure the binding affinity to amyloid fibers or oligomers, and/or an activity assay.

16. The method of claim 2, further comprising deriving on a computer a refined pharmacophore based on the identified candidate amyloid inhibitors.

17. Starting with the refined pharmacophore derived in claim 16, testing a new set of candidate amyloid binders by repeating the docking and selecting steps, and testing the candidate amyloid binders for their ability to inhibit amyloid-mediated cell toxicity, in order to identify a further refined pharmacophore.

18. Starting with the further refined pharmacophore derived in claim 17, repeating the docking and screening steps, and testing the candidate amyloid binders for their ability to inhibit amyloid-mediated cell toxicity in order to identify a yet further refined pharmacophore; and repeat.

19. A pharmaceutical composition comprising one or more of the compounds BAF4, BAF8, BAF11, BAF12, BAF14, BAF30 or BAF31, as shown in FIG. 18, or the derivatives of BAF11—the isomer, σR1, σR3 or ΔOHσR—as shown in FIG. 19, or the derivative of BAF30-αR1—as shown in FIG. 20, or a pharmaceutically acceptable salt, hydrate, solvate or metal chelate thereof, and a pharmaceutically acceptable carrier.

20. The pharmaceutical composition of claim 19, wherein the compound is detectably labeled.

21. The pharmaceutical composition of claim 20, wherein the label is a radioactive or fluorescent label.

22. The pharmaceutical composition of claim 5, wherein the label is suitable for detection by PET.

23. A method for determining the presence of Aβ or tau oligomers or fibers in a sample, comprising contacting a sample suspected of comprising such fibers with an effective amount of one or more of BAF4, BAF8, BAF11, BAF12, BAF14, BAF30 or BAF31, as shown in FIG. 18, or the derivatives of BAF11—the isomer, σR1, σR3 or ΔOHσR—as shown in FIG. 19, or the derivative of BAF30σR1—as shown in FIG. 20, or a pharmaceutically acceptable salt, hydrate, solvate or metal chelate thereof, wherein the compound is detectably labeled; and
measuring the amount of bound label in the sample,
wherein a statistically significantly higher amount of label than that in a control sample lacking the fibers indicates the presence of the fibers in the sample.

24. The method of claim 23, which is carried out in vitro or in vivo.

25. The method of claim 23, which is a method for diagnosing the presence of an amyloid disease.

26. The method of claim 23, which is a method for diagnosing Alzheimer's disease.

27. A method for detecting the presence of Aβ or tau fibers in a subject, comprising introducing into the subject a compound with an effective amount of one or more of the compounds BAF4, BAF8, BAF11, BAF12, BAF14, BAF30 or BAF31, as shown in FIG. 18, or the derivatives of BAF11—the isomer, σR1, σR3 or ΔOHσR—as shown in FIG. 19, or the derivative of BAF30-αR1— as shown in FIG. 20, or a pharmaceutically acceptable salt, hydrate, solvate or metal chelate thereof, wherein the compound is labeled with a nuclide that can be detected by PET; and
measuring the amount of bound label in the brain by PET,
wherein a statistically significantly higher signal than that in a control sample lacking the fibers indicates the presence of the fibrils in the brain of the subject.

28. A method for reducing or inhibiting amyloid-based cellular toxicity, comprising contacting amyloid protofilaments with an effective amount of BAF4, BAF8, BAF11, BAF12, BAF14, BAF30 or BAF31, as shown in FIG. 18, or the derivatives of BAF11—the isomer, σR1, σR3 or ΔOHσR—as shown in FIG. 19, or the derivative of BAF30-σR1— as shown in FIG. 20, or a pharmaceutically acceptable salt, hydrate, solvate or metal chelate thereof.

29. The method of claim 28, which is carried out in vitro.

30. The method of claim 28, which is carried out in vivo.

31. A method for treating an amyloid-mediated disease or condition, comprising administering to a subject having or likely to have the disease or condition an effective amount of BAF4, BAF8, BAF11, BAF12, BAF14, BAF30 or BAF31, as shown in FIG. 18, or the derivatives of BAF11—the isomer, σR1, σR3 or ΔOHσR—as shown in FIG. 19, or the derivative of BAF30-αR1— as shown in FIG. 20, or a pharmaceutically acceptable salt, hydrate, solvate or metal chelate thereof.

32. A computer readable medium providing the structural representation of a co-crystal of a protofilament of an amyloid protein with a small molecule that is known to bind to the amyloid protein.

33. A kit for detecting the presence of Abeta or tau in a sample, comprising a compound of the invention in a container.
